(19) United States
(12) Patent Application Publication
(10) Pub. No.: US 2012/0053427 A1
(43) Pub. Date: Mar. 1, 2012

(54) OPTICAL SENSOR CONFIGURATION AND METHODS FOR MONITORING GLUCOSE ACTIVITY IN INTERSTITIAL FLUID

(75) Inventors: David R. Markle, Berwyn, PA (US); Soya Gamsey, Huntington Beach, CA (US); Thomas A. Peyser, Menlo Park, CA (US); William Markle, Laguna Niguel, CA (US)

(73) Assignee: GLUMETRICS, INC., Irvine, CA (US)

(21) Appl. No.: 13/221,696

(22) Filed: Aug. 30, 2011

Related U.S. Application Data
(60) Provisional application No. 61/378,728, filed on Aug. 31, 2010.

(51) Int. Cl. A61B 5/1459; A61B 5/01

(52) U.S. Cl. 600/301; 600/342

(57) ABSTRACT

Embodiments of the invention are directed to an optical sensor for detecting blood glucose by deploying the optical sensor into the interstitial fluid. The sensor comprises a chemical indicator system capable of generating an optical signal related to the blood glucose activity. The sensor further comprises a means for generating and detecting an optical reference signal unrelated to the blood glucose activity, such that ratiometric correction of blood glucose measurements for artifacts in the optical system is enabled.

### CROSS-REFERENCE TO RELATED APPLICATIONS

This application claims priority to U.S. Provisional Patent Application No. 61/378,728, filed Aug. 31, 2010 the disclosure of which is hereby expressly incorporated by reference and hereby expressly made a portion of this application.

### BACKGROUND OF THE INVENTION

1. Field of the Invention

Embodiments of the invention are directed to an optical sensor for detecting an analyte, for example bioavailable glucose (glucose activity'), in the blood via interstitial fluid contact. In preferred embodiments, the sensor comprises an optical fiber having a fluorescence chemistry disposed along the distal region of the fiber, wherein at least the distal region of the fiber is configured for deployment within the interstitial space.

2. Description of the Related Art

Hyperglycemia and insulin resistance are common in critically ill patients, even if such patients have not previously had diabetes. In these situations, glucose levels rise in critically ill patients thereby increasing the risk of damage to a patient's organs. Further, studies have shown that normalization of blood glucose levels with insulin therapy improves the prognosis for such patients, thereby decreasing mortality rates.

Recent scientific evidence confirms that dramatic improvements in the clinical outcome of hospitalized Intensive Care Unit (ICU) patients can result from therapeutic control of blood glucose to normal ranges. These studies indicate that glycemic control (GC) of ICU patients may reduce mortality by as much as 40%, and significantly lower complication rates. In these situations, it is necessary to accurately, conveniently and continuously monitor blood sugar in a real-time device specifically designed to meet the challenging needs of the ICU environment.

While clinicians have used insulin for decades to regulate glucose levels in diabetics, determining precise dosages remains a problem. Dosage protocols for insulin attempt to replicate the physiologic secretion of the hormone by the pancreas. However, administering according to fixed times and algorithms based on blood glucose measurements can only crudely approximate the ability of a healthy individual to continuously adjust insulin production in response to the amount of bioavailable glucose and the needs of the body. Thus, to determine the precise amount of insulin that should be administered to maintain a patient's blood glucose at an appropriate level, it is necessary to have near real-time, accurate measurements of the amount of bioavailable glucose circulating in blood.

Unfortunately, existing methods for determining blood glucose concentrations fail to provide near real-time, accurate measurements of the amount of bioavailable glucose. Clinicians and diabetic patients typically rely on point-of-care testing that seems to measure glucose concentration in plasma, e.g., using glucometers to read test strips that filter separate plasma from cells in a drop of whole blood. While the results can be available quickly, they vary depending on the patient's hematocrit, plasma protein and lipid profiles, etc., and can often be falsely elevated (See e.g., Chakravarthy et al., 2005 “Glucose determination from different vascular compartments by point-of-care testing in critically ill patients” Chest 128(4) October, 2005 Supplement: 220S-221S). More accurate determinations can be obtained by first separating the cellular components of whole blood. However, this requires separation of the plasma from the cellular components of blood, e.g., by centrifugation. Subsequently, isolated plasma must be stored and/or transported and/or diluted prior to analysis. Storage and processing conditions, e.g., temperature, dilution, etc., will almost certainly perturb the in vivo equilibrium between the bound and free (bioavailable) glucose. Consequently, regardless of the technology subsequently employed for measuring plasma glucose concentration (e.g., glucose oxidase, mass spectrometry, etc.), the measured glucose concentration is likely no longer reflective of the amount of bioavailable glucose in vivo. Therefore, it is not feasible to use plasma glucose measurements for near real-time monitoring and adjustment of a patient's glucose level.

Data presented to the FDA advisory committee meeting consisted of studies validating the correlation between the measurements of glucose in interstitial fluid with the blood glucose measurements made with home monitoring devices. (CGMS: FDA Summary of Safety and EffectivenessF, PMA No. 980022, approval letter issued on Jun. 15, 1999; GlucoWatch G2 Biographer: FDA Summary of Safety and Effectiveness, PMA No. 990026/S008, approval letter issued on Aug. 26, 2002; Tamada J A, Garg S, Jovanovic L et al. Noninvasive glucose monitoring: comprehensive clinical results. J. Amer. Med. Assoc. 1999; 282:1839-44). While the individual values between the two may vary, in general the panel found that the overall trends in glucose levels detected by frequent measurements produced potentially clinically important information.

Accordingly, there remain important and unmet needs to accurately measure in near real-time the amount of bioavailable glucose within a clinically relevant glucose activity range by measuring interstitial glucose levels to determine the amount of bioavailable glucose in the blood. Then one can utilize this measurement to manually or automatically adjust the glucose activity by administration of a glucose modulator (e.g., insulin) to accomplish glycemic control.

### SUMMARY OF THE INVENTION

A method is disclosed for monitoring blood glucose in a subject. The method comprises: providing a glucose sensor, comprising: an optical fiber configured for subcutaneous deployment and capable of propagating light along a light path, and further comprising an equilibrium, non-consuming chemical indicator system disposed within the light path of the optical fiber, wherein the chemical indicator system comprises a fluorophore capable of generating a fluorescent emission signal in response to an excitation light signal, and a glucose binding moiety operably associated with the fluorophore and adapted to modify the intensity of the fluorescent emission signal in relation to the amount of glucose bound; deploying the glucose sensor into subcutaneous tissue of the subject; interrogating the chemical indicator system with an excitation light signal; and detecting the intensity of the fluorescent emission light signal.

In one embodiment, the method further comprises: obtaining a blood sample from the subject; measuring the glucose concentration of the blood sample independent of the chemical indicator system; calculating a correction factor by comparing the first emission light signal with the glucose concentration measured independently of the chemical indicator system; and adjusting the blood glucose concentration measurement of the chemical indicator system with the correction factor.

In preferred embodiments, the distal end of the glucose sensor comprises an atraumatic tip portion formed from at least one material selected from the group consisting of plastics, polymers, gels, metals and composites. The atraumatic tip portion is preferably configured to reduce trauma within the subcutaneous tissues and may have a shape selected from the group consisting of hemispherical, parabolic, and elliptical.

In a variation to the method, the chemical indicator system is further immobilized by a hydrogel within a gap in the optical fiber.

Preferably, the glucose binding moiety comprises: a viologen quencher capable of quenching the emission intensity of the fluorophore; and a benzyl boronic acid group capable of binding glucose, wherein the benzyl boronic acid group is coupled to the viologen quencher, such that the degree of emission quenching is related to the amount of glucose binding.

In some embodiments, the glucose sensor further comprises a reference material, and the method further comprises; reflecting a portion of the excitation light signal off of the reference material to generate a reflected portion of the excitation light signal; and detecting the reflected portion of the excitation light signal.

In a variation, the reference material comprises a second fluorophore, and the method further comprises: interrogating the reference material with the excitation light signal such that the reference material generates a second emission light signal, wherein the intensity of the second emission light signal is not related to the amount of glucose bound; and detecting the second emission light signal. In another variation, the reference material is encased in a glucose impermeable membrane.

A sensor for detecting an analyte concentration in a blood vessel is disclosed in accordance with an embodiment of the invention. The sensor comprises: an optical fiber with proximal and distal ends; an atraumatic tip portion with proximal and distal ends, wherein the proximal end of the atraumatic tip portion is separated from the distal end of the optical fiber, such that a gap exists between the atraumatic tip portion and the optical fiber; a rod with proximal and distal ends, wherein the proximal end of the rod is attached to the distal end of the optical fiber, and wherein the distal end of the rod is attached to the proximal end of the atraumatic tip portion, such that the rod traverses the gap and couples the optical fiber to the atraumatic tip portion; a chemical indicator system capable of generating an emission light signal in response to an excitation light signal, wherein the intensity of the emission light signal is related to the analyte concentration, and wherein the chemical indicator system is disposed within the gap; and a selectively permeable membrane disposed over the gap, wherein the sensor is sized for deployment within the blood vessel.

In one variation to the analyte sensor, the chemical indicator system is immobilized within the gap by a hydrogel. In another variation, the sensor further comprises a temperature sensor. The optical fiber preferably has a diameter of between about 0.005 inches and about 0.020 inches. In another variation, the sensor further comprises a reflective region. Preferably, the reflective region comprises a reflective surface of the proximal end of the rod. In one embodiment, the rod may be attached to the optical fiber and atraumatic tip portion by heating. In another embodiment, the rod may be attached to the optical fiber by a reflective or optically clear adhesive.

In variations to the sensor, the shape of the distal end of the atraumatic tip portion may be configured to reduce trauma within the blood vessel. In various embodiments, the shape of the distal end of the atraumatic tip portion may be selected from the group consisting of hemispherical, parabolic, and elliptical. In another variation, the distal end of the atraumatic tip portion is flexible. In another variation, the distal end of the atraumatic tip portion is deformable. The distal end of the atraumatic tip portion may be formed from at least one material selected from the group consisting of plastics, polymers, gels, metals and composites.

The rod may be formed from at least one material selected from the group consisting of metal, metal alloy, plastic, polymer, ceramic, and composite material. In a preferred variation, the rod is formed from stainless steel, titanium, or Nitinol. In one embodiment, the rod is cylindrical. Preferably, the rod diameter is between about 0.002 inches and about 0.010 inches. The rod may be flexible in some embodiments. The rod is stiffer than the optical fiber in some embodiments. In such embodiments, the rod is preferably sufficiently stiff to prevent flexing of the sensor along the gap.

A sensor for detecting an analyte concentration in a blood vessel is disclosed in accordance with another embodiment of the present invention. The sensor comprises: an optical fiber with proximal and distal ends; an atraumatic tip portion with proximal and distal ends, wherein the proximal end of the atraumatic tip portion is separated from the distal end of the optical fiber, such that a gap exists between the atraumatic tip portion and the optical fiber; a hypotube with proximal and distal ends, wherein the proximal end of the hypotube is attached to the distal end of the optical fiber, and wherein the distal end of the hypotube is attached to the proximal end of the atraumatic tip portion, such that the hypotube traverses the gap and couples the optical fiber to the atraumatic tip portion, wherein the hypotube comprises at least one window that opens onto the gap; a chemical indicator system capable of generating an emission light signal in response to an excitation light signal, wherein the intensity of the emission light signal is related to the analyte concentration, and wherein the chemical indicator system is disposed within the gap; and a selectively permeable membrane disposed over the at least one window, wherein the sensor is sized for deployment within the blood vessel. In preferred embodiments, the chemical indicator system is immobilized by a hydrogel within the cavity formed within the hypotube. In further preferred embodiments of the sensor with hypotube, a reflective member is disposed within the sensor. In further preferred embodiments of the sensor with hypotube, a fluorescent member is disposed within the sensor.

A sensor for detecting an analyte concentration in a blood vessel is disclosed according to another embodiment of the present invention. The sensor comprises: an optical fiber with proximal and distal ends; an atraumatic tip portion with proximal and distal ends, wherein the proximal end of the atraumatic tip portion is separated from the distal end of the optical fiber, such that a gap exists between the atraumatic tip portion and the optical fiber; a cage connecting the optical fiber and atraumatic tip portion, wherein the optical fiber is at least partially enclosed within the cage, and wherein the cage has at least one window; a chemical indicator system disposed within the cage, wherein the chemical indicator system is adjacent the window and is separated from analyte by a selectively permeable membrane, and wherein the chemical indicator system is capable of generating an emission light signal in response to an excitation light signal, wherein the intensity of the emission light signal is related to the analyte concentration; and a reference material, wherein the reference material is configured to either reflect a portion of the excitation light signal before the excitation light signal enters the chemical indicator system or to return a second emission light signal, wherein the intensity of the second emission light signal is not related to the analyte concentration.

In another embodiment, a method for measuring glucose concentration is provided. The method comprises transmitting a first light in a first direction through an optical fiber to a glucose sensor, where the glucose sensor comprises a hydrogel cavity having a fluorophore system. At least a portion of the first light is reflected off a reflective surface of the glucose sensor as a second light in a second direction opposite the first direction. A third light is emitted in the second direction. The third light results from the chemical indicator system fluorescing. The method further comprises calculating the glucose concentration, where the glucose concentration is determined by the ratio of the emitted third light to the reflected second light. The ratio is independent of the intensity of the first light.

In another embodiment, a method for measuring glucose concentration is provided. The method comprises transmitting a first light in a first direction through an optical fiber to a glucose sensor, where the glucose sensor comprises a hydrogel cavity having a fluorophore system which is sensitive to glucose as well as a second fluorophore which is glucose insensitive. A second light from the glucose sensitive fluorophore is emitted in a second direction opposite the first direction. A third light from the glucose insensitive fluorophore is also emitted in the second direction. The method further comprises calculating the glucose concentration, where the glucose concentration is determined by the ratio of the emitted second light to the emitted third light. The ratio is independent of the intensity of the first light.

In another embodiment, a system for measuring glucose is provided. The system comprises at least one light source, at least one optical fiber coupled to the light source, any one of the glucose sensors described above coupled to the optical fiber, and a data processing device coupled to the glucose sensor.

In another embodiment, a method for manufacturing a glucose sensor is provided. The method comprises inserting a first end of a rod into an optical fiber, inserting a second end of the rod into an atraumatic tip, surrounding the rod with a hydrogel cavity, and enclosing the hydrogel cavity with a selectively permeable membrane.

In another embodiment, a method for manufacturing a glucose sensor is provided. The method comprises cutting a window in a hypotube, contacting an optical fiber with a first end of the hypotube, and heating the optical fiber, such that the optical fiber swells to fully contact the first end of the rod.

In another embodiment, a sensor for detecting an analyte concentration in a blood vessel is provided. The sensor comprises an optical fiber with a proximal end and a distal end. The distal end of the optical fiber comprises a glucose sensing hydrogel. The glucose sensing hydrogel comprises a first fluorophore, a quencher, and at least one glucose receptor. A reference fiber is adjacent the optical fiber and has a proximal end and a distal end. The distal end of the reference fiber comprises a reference material. The reference material comprises a second fluorophore. A light emitting diode is operably coupled to the glucose fiber and the reference fiber. The light emitting diode sends an excitation light to the glucose fiber and the reference fiber. A glucose signal detector is operatively coupled to the glucose fiber. The glucose signal detector receives a first fluorescent light from the glucose fiber. A reference signal detector is operatively coupled to the reference fiber. The reference signal detector receives a second fluorescent light from the reference fiber.

### BRIEF DESCRIPTION OF THE DRAWINGS

FIG. 1A shows a glucose sensor having a series of holes that form a helical configuration.

FIG. 1B shows a glucose sensor having a series of holes drilled or formed at an angle.

FIG. 1C shows a glucose sensor having at least one spiral groove.

FIG. 1D shows a glucose sensor having a series of triangular wedge cut-outs.

FIG. 2A shows a cross-sectional view of one embodiment of a glucose sensor having a cavity in the distal portion of the sensor and a temperature probe.

FIG. 2B shows a perspective view of the glucose sensor shown in FIG. 2.

FIG. 3A shows a cross-sectional view of another embodiment of a glucose sensor having a cavity in the distal portion of the sensor.

FIG. 3B shows a perspective view of the glucose sensor shown in FIG. 4.

FIG. 4 shows a cross-sectional view of another embodiment of a glucose sensor having a window opening to a cavity in the distal portion of the sensor.

FIG. 5 shows a cross-sectional view of another embodiment of a glucose sensor having a cavity in a distal portion of the sensor enclosed within a cage and an additional reference material.

FIG. 6 shows a cross-sectional view of another embodiment of a glucose sensor having a cavity in a distal portion of the sensor and an additional reference material.

FIG. 7 shows a cross-sectional view of another embodiment of a glucose sensor having a cavity in a distal portion of the sensor enclosed within a cage and a reference material extending to the atraumatic tip.

FIG. 8 shows a cross-sectional view of another embodiment of a glucose sensor having a cavity in a distal portion of the sensor enclosed within a cage and a reference material as a bar extending across the diameter of the cage.

FIG. 9 shows a cross-sectional view of another embodiment of a glucose sensor having a cavity in a distal portion of the sensor enclosed within a reference material, further enclosed within a cage.

FIG. 10 shows a cross-sectional view of another embodiment of a glucose sensor having a cavity in the distal portion of the sensor enclosed within a cage and a reference material as a bar embedded within the optical fiber.

FIG. 11 shows a cross-sectional view of another embodiment of a glucose sensor having a cavity and a reference material side-by-side in the distal portion of the sensor enclosed within a cage.

FIG. 12 shows a cross-sectional view of another embodiment of a glucose sensor having a cavity in the distal portion of the sensor enclosed within a cage and a translucent reference material between the optical fiber and cavity.

FIG. 13 shows a schematic view of another embodiment of a glucose sensor having a glucose sensing optical fiber adjacent to a reference optical fiber.

FIG. 14 shows a glucose measurement system comprising one excitation light source, a single exciter-dual emitter fluorophore system, and a microspectrometer and/or spectrometer.

FIG. 15 shows the Stern-Volmer quenching of HPTS-CysMA/3,3′-oBBV in solution.

FIG. 16 shows the glucose response of HPTS-CysMA/3,3′-oBBV in solution.

FIG. 17 shows the glucose response of HPTS-CysMA/3,3′-oBBV in hydrogel.

FIG. 18 shows the glucose response of a sensor deployed into interstitial space for about 8 hours.

FIG. 19 shows the glucose response of a sensor deployed into interstitial space for about 24 hours.

### DETAILED DESCRIPTION OF THE PREFERRED EMBODIMENT

Various embodiments of optical systems and methods are disclosed herein for determining a glucose concentration within an interstitial fluid—i.e. an interstitial glucose concentration. The various embodiments preferably share at least two features. First, they involve exciting a chemical indicator system with an excitation light signal and measuring the emission light signal of the indicator system, wherein the indicator system is in contact with the interstitial fluid and comprises a fluorescent dye operably coupled to a glucose binding moiety—such that the emission light signal generated by the indicator system upon excitation is related to the interstitial glucose concentration. Second, they involve correcting the interstitial glucose concentration measurements from the indicator system for potential artifacts due to the optical system, which artifacts are unrelated to the interstitial glucose concentration. The correction is performed by ratiometric analysis. More particularly, the ratio of emission light signal to a second light signal that is propagated through the optical system, e.g., the excitation light signal or a separate reference light signal, is used for correcting any non-glucose related contributions of the optical system. Where the excitation light signal is used for the ratiometric correction, the sensor preferably includes a reflective surface, e.g., a mirror, located somewhere along the sensor, such that at least a portion of the excitation light which has passed through the optical system is reflected back to a detector. Where a separate reference light signal is used, the reference light signal may either be: (1) generated by a separate light source and reflected back to a detector, or (2) generated as a separate emission light signal from a separate dye disposed somewhere along the sensor. Thus, a glucose sensor in accordance with preferred embodiments of the present invention will comprise either a reflective surface or a second dye adapted to emit a reference light signal. In other embodiments, the glucose sensor can have a biocompatible coating such as an antithrombogenic coating. In yet other embodiments, the glucose concentration is calculated using a modified Michaelis-Menten equation.

Various structural configurations have been proposed for (1) holding a chemical indicator system in a position disposed within the excitation light path which exposes the chemical indicator system to the interstitial fluid, (2) for introducing an excitation light signal to the indicator system, (3) for detecting an emission light signal from the indicator system, and (4) for enabling ratiometric correction of glucose determinations for artifacts to the system optics; see in particular U.S. Patent Publication No. 2008/0188725 and 2011/0105866, both of which are incorporated herein their entireties by reference. More particularly, aspects of the present invention relate to improvements and alternative embodiments for generating a reference light signal (as discussed in 2008/0188725), either through various mirror/reflective surface configurations adapted to return a portion of the excitation light signal back to a detector or through generating a separate emission light signal from a separate dye. Aspects of the present invention relate to new and improved configurations for disposing a chemical indicator system within an interrogation light path, wherein the sensor is more robust and exhibits improved patient tolerance.

Optical glucose sensors, such as those described in U.S. Pat. Nos. 5,512,246, 5,503,770, 6,627,177, 7,417,164 7,470,420, 7,751,863, 7,767,846, 7,824,918, 7,829,341 and 7,939,664, U.S. Patent Publication Nos. 2008/0188725, 2008/0187655, 2009/0018426, 2009/0018418, 2009/0264719, 2009/0177143, 2010/0312483, 2011/0077477, 2011/0105866, and 2011/0152658, PCT Publication Nos. WO2008/001091, WO2009/019470, WO2009/087373, WO2009/106805, WO2011/084713, and WO2011/075711, co-pending U.S. patent application Ser. Nos. 13/022,494 and 13/046,571 (each of which is incorporated herein in its entirety by reference thereto) typically employ a chemical indicator system disposed at the distal end of an optical fiber, wherein the indicator system is maintained in contact with the blood, such that an excitation light signal sent distally down the fiber causes the chemical indicator system to emit a light signal related to the concentration of glucose.

In certain embodiments, an optical glucose measurement system is disclosed for measuring glucose concentration in interstitial fluid using one or more glucose-sensing chemical indicator systems. Such indicator systems preferably comprise a fluorophore operably coupled to a glucose binding moiety. Preferably, the glucose binding moiety acts as a quencher with respect to the fluorophore (e.g., suppresses the fluorescent emission signal of the fluorophore in response to excitation light when it associates with the fluorophore). In preferred embodiments, as the glucose binding moiety binds glucose (e.g., as glucose concentrations rise), it dissociates from the fluorophore, which then generates a fluorescent emission signal upon excitation. Accordingly, in such embodiments, the higher the glucose concentration, the more glucose bound by the binding moiety, the less quenching, and the higher the fluorescence intensity of the fluorophore upon excitation.

In certain embodiments, the optical glucose measurement system measures glucose concentrations intravascularly and in real-time through the use of such chemical indicator systems. In certain embodiments, the glucose-sensing chemical indicator systems are immobilized in a hydrogel. The hydrogel may be inserted into an optical fiber path such that light may be transmitted through the hydrogel while at least a portion of the hydrogel is in contact with interstitial fluid. The hydrogel is preferably permeable to blood and analytes, specifically glucose. In certain embodiments, the optical fiber together with the hydrogel comprises a glucose sensor that is placed in mammalian (human or animal) tissue such that the sensing elements of the glucose sensor are bathed in interstitial fluid of the tissue.

Examples of glucose-sensing chemical indicator systems and glucose sensor configurations for intravascular glucose monitoring include the optical sensors disclosed in U.S. Pat. Nos. 5,137,033, 5,512,246, 5,503,770, 6,627,177, 7,417,164, 7,470,420, 7,751,863, 7,824,918, 7,829,341, and 7,939,664, U.S. Patent Publ. Nos. 2008/0188725, 2008/0187655, 2009/0018426, 2009/0018418, 2009/0177143, and 2009/0264719, and U.S. Provisional Appl. No. 61/508,509, each of which is incorporated herein in its entirety by reference thereto. In certain embodiments, these optical sensors may be used for interstitial glucose monitoring, in addition to their use intravascularly.

Light may be transmitted into an optical glucose sensor from a light source. In certain embodiments, the light source is a light emitting diode that emits an optical excitation signal. The optical excitation signal excites the fluorophore system(s), such that the fluorophores emit light at an emission wavelength. In certain embodiments, the fluorophore systems are configured to emit an optical emission signal at a first wavelength having an intensity related to the blood glucose concentration in the interstitial fluid. In certain embodiments, light is directed out of the glucose sensor such that the light is detected by at least one detector. The at least one detector preferably measures the intensity of the optical emission signal, which is related to the glucose concentration present in the interstitial fluid. Various optical configurations for interrogating glucose-sensing chemical indicator systems with one or more excitation light signals and for detecting one or more emission light signals from the chemical indicator systems may be employed, see e.g., U.S. Pat. No. 7,751,863, US Publication No. 2008/0188725 and 2011/0105866, incorporated herein in its entirety by reference thereto.

### Glucose Activity and Glycemic Control

While clinicians have used insulin for decades to regulate glucose levels in diabetics and critically ill patients, determining precise dosages remains a problem. Insulin reduces circulating glucose levels through a series of complex interactions involving a number of hormones and cell types. Dosage protocols for insulin attempt to replicate the physiologic secretion of the hormone by the pancreas. However, administering according to fixed times and algorithms based on blood glucose measurements can only crudely approximate the ability of a healthy individual to continuously adjust insulin production in response to the amount of bioavailable glucose and the needs of the body. Thus, to determine the precise amount of insulin that should be administered to maintain a patient's blood glucose at an appropriate level, it is necessary to have near real-time, accurate measurements of the amount of bioavailable glucose circulating in blood.

Unfortunately, most existing methods for determining blood glucose concentrations fail to provide near real-time, accurate measurements of the amount of bioavailable glucose. Clinicians and diabetic patients typically rely on point-of-care testing that seems to measure glucose concentration in plasma, e.g., using glucometers to read test strips that filter separate plasma from cells in a drop of whole blood. While the results can be available quickly, they vary depending on the patient's hematocrit, plasma protein and lipid profiles, etc., and can often be falsely elevated (See e.g., Chakravarthy et al., 2005 “Glucose determination from different vascular compartments by point-of-care testing in critically ill patients” Chest 128(4) October, 2005 Supplement: 220S-221S). More accurate determinations can be obtained by first separating the cellular components of whole blood. However, this requires separation of the plasma from the cellular components of blood, e.g., by centrifugation. Subsequently, isolated plasma must be stored and/or transported and/or diluted prior to analysis. Storage and processing conditions, e.g., temperature, dilution, etc., will almost certainly perturb the in vivo equilibrium between the bound and free (bioavailable) glucose. Consequently, regardless of the technology subsequently employed for measuring plasma glucose concentration (e.g., glucose oxidase, mass spectrometry, etc.), the measured glucose concentration is likely no longer reflective of the amount of bioavailable glucose in vivo. Therefore, it is not feasible to use plasma glucose measurements for near real-time monitoring and adjustment of a patient's glucose level.

Accordingly, in certain embodiments, the preferred glucose sensors described herein measure glucose “activity” as opposed to glucose concentration. More precisely, glucose activity refers to the amount of free glucose per kilogram of water. In some embodiments, glucose activity can be measured directly using glucose sensors, such as the equilibrium, non-consuming optical glucose measurement systems discussed above, which employ a chemical indicator system to quantify the amount of free, bioavailable glucose, which is in equilibrium between the water compartment of blood (i.e., not associated with cells, proteins or lipids, etc.) and the glucose binding moiety/quencher. The discussion of the sensors that follow will often refer to the physical quantity to be measured as an “analyte concentration”, “glucose concentration” or simply a “concentration.” However, it is to be understood that “concentration” as used herein, refers to both “analyte concentration” as that phrase would be ordinarily used and also to “activity” (in some cases “glucose activity”) as that phrase is described above.

### Optical Glucose Sensor Configurations

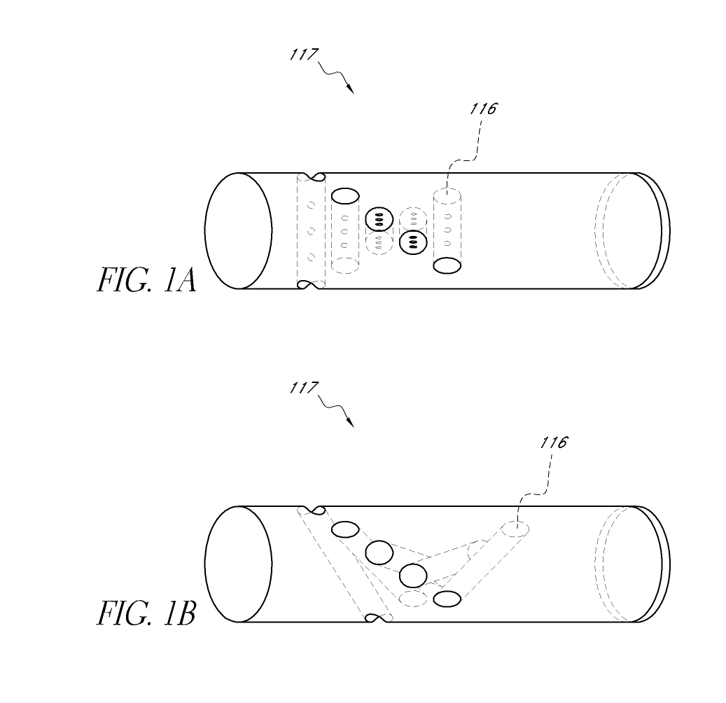

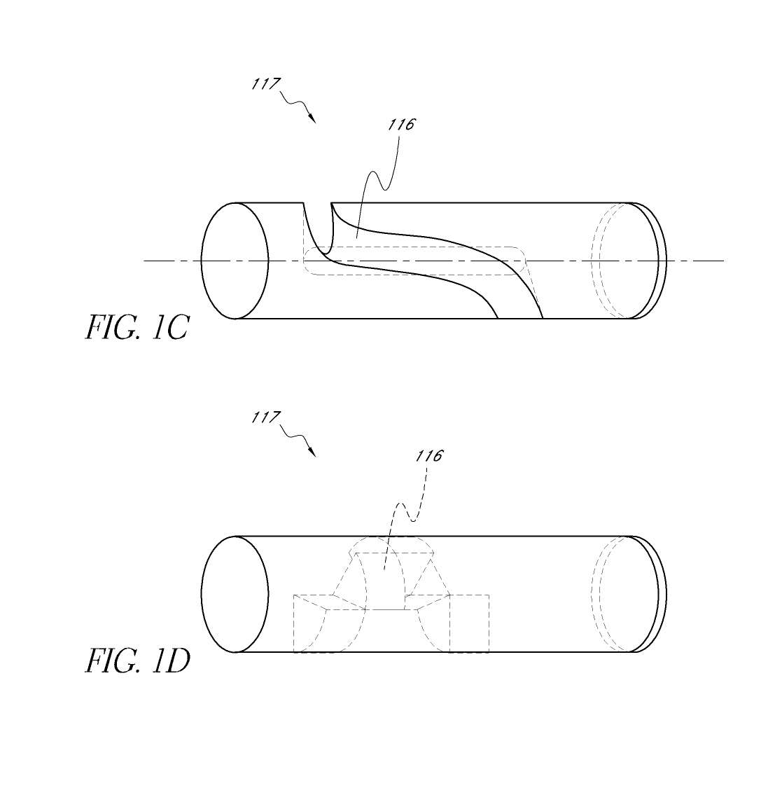

With reference to FIGS. 1A-D, certain prior art embodiments (see US Patent Publication No. 2008/0188725) are illustrated. The glucose sensor 117 in FIG. 1A is an optical fiber with a series holes 116 drilled straight through the sides of the optical fiber. In certain embodiments, the holes 116 are filled with one or more glucose-sensing chemical indicator systems. These holes may be covered with a selectively permeable membrane, wherein the permeability is selected such that the molecules of the chemical indicator system (e.g., fluorophore and quencher) are retained within the cavities, whereas glucose is freely permeable. In certain embodiments, the series of holes 116 that are drilled through the glucose sensor 117 are evenly spaced horizontally and evenly rotated around the sides of the glucose sensor 117 to form a spiral or helical configuration. In certain embodiments, the series of holes are drilled through the diameter of the glucose sensor.

With reference to FIG. 1B, in certain embodiments, the glucose sensor 117 is a solid optical fiber with a series of holes 116 drilled through the sides of the fiber at an angle. In certain embodiments, the series of holes drilled at an angle, which are filled with hydrogel/chemical indicator system, are evenly spaced horizontally and evenly rotated around the sides the glucose sensor 117. With reference to FIG. 1C, in certain embodiments, the optical fiber comprises a groove 116 along the length of the optical fiber, wherein the groove is filled with hydrogel/chemical indicator system. In certain embodiments, the depth of the groove extends to the center of the optical fiber. In certain embodiments, the groove spirals around the optical fiber. In certain embodiments, the groove spirals around the optical fiber to complete at least one rotation. In certain embodiments, the groove spirals around the optical fiber to complete multiple rotations around the optical fiber.

With reference to FIG. 1D, in certain embodiments, the glucose sensor 117 is a solid optical fiber with triangular wedges 116 cut from the fiber. In certain embodiments, the triangular wedge areas are filled with hydrogel/chemical indicator system. In certain embodiments, the triangular wedges cut-outs are evenly spaced horizontally and around the sides of the glucose sensor 117. In certain embodiments, all light traveling in the glucose sensor 117 is transmitted through at least one hole or groove 116 filled with hydrogel/chemical indicator system.

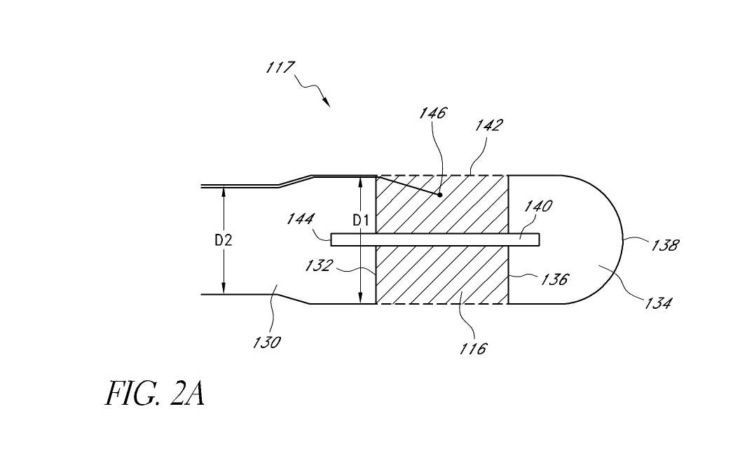

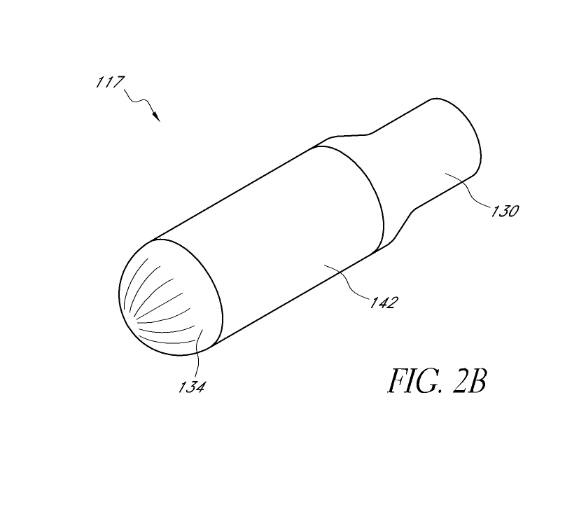

In certain embodiments, as illustrated in FIGS. 2-6, the glucose sensor 117 comprises an optical fiber 130 having a distal end 132, an atraumatic tip portion 134 having a proximal end 136 and a distal end 138, a void or cavity 116 between the distal end 132 of the optical fiber 130 and the proximal end 136 of the atraumatic tip portion 134, and a rod 140 connecting the distal end 132 of the optical fiber 130 to the proximal end 136 of the atraumatic tip portion 134, wherein the rod traverses the void or cavity. In preferred embodiments, molecules of a chemical indicator system are disposed within the void or cavity 116 and immobilized (by covalent bonding or non-covalent interaction) or otherwise associated within hydrogel matrices. See e.g., the chemical indicator systems disclosed in U.S. Pat. Nos. 7,417,164 and 7,470,420. The cavity 116 may be loaded with hydrogel/chemical indicator system by any methods known in the art. In preferred embodiments, the cavity 116 is filled with hydrogel/chemical indicator system in a liquid state. The hydrogel/chemical indicator systems are preferably polymerized in situ, as detailed in co-pending US Patent Publication No. 2008/0187655.

In certain embodiments, the rod 140 is attached to the optical fiber 130 and/or atraumatic tip 134 by heating and expanding the optical fiber 130 and atraumatic tip 134 and embedding the rod 140 there between. In certain embodiments, the optical fiber 130 is heated to between about 100° C. and about 160° C., more preferably between about 110° C. and about 140° C. In other embodiments, the optical fiber 130 is first heated and then cooled one or more times. In certain embodiments, the rod 140 is attached to the optical fiber 130 and/or the atraumatic tip 134 by applying an adhesive. In preferred embodiments, the adhesive is biocompatible, such as for example, cyanoacrylates, epoxies, light cure adhesives, silicones, and urethanes. In certain embodiments, after applying the adhesive and joining the rod 140 with the optical fiber 130 and atraumatic tip 134, the adhesive is cured at room temperature, by heating, or by applying UV/visible light. In certain embodiments, the time to fix the rod 140 to the optical fiber 130 and/or atraumatic tip 134 can vary from about 5 seconds to about 60 seconds, from about 15 minutes to about 5 hours, from about 60 seconds to about 10 minutes, or up to about 24 hours.

In some embodiments, the proximal surface of the rod 144 is reflective so that a portion of the excitation light signal (or reference light signal) is reflected proximally down the optical fiber 130 to a detector (not shown). The term rod is used herein to refer to any elongate structural member, regardless of its geometry, configured to connect the atraumatic tip portion to the optical fiber. The rod may be centered coaxially (as illustrated) or off-centered with regard to the cross-section of the fiber and atraumatic tip portion. In some embodiments, there may be more than one rod extending between the fiber and the atraumatic tip portion. Where more than one rod is employed, the rods may be arranged symmetrically or asymmetrically with respect to the cross-section of the fiber and atraumatic tip portion.

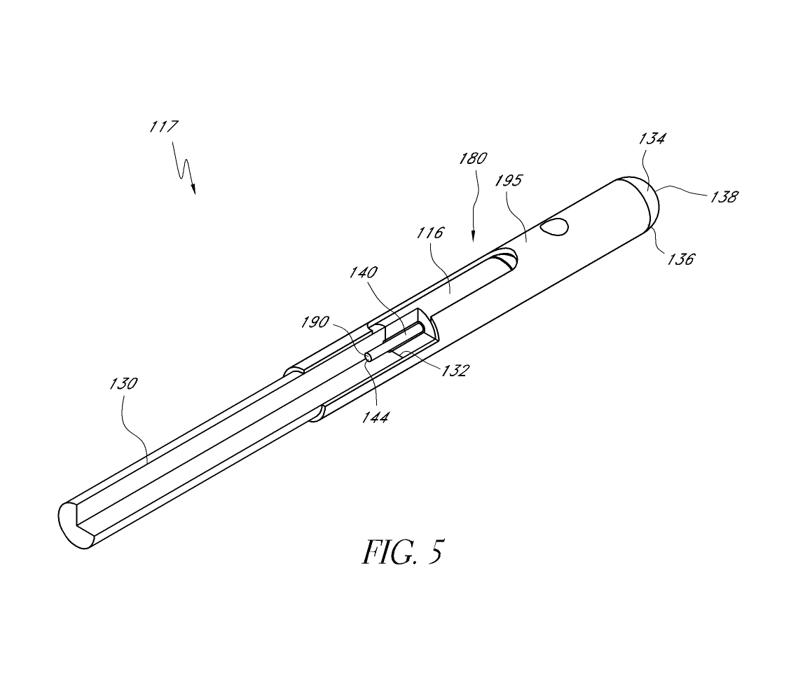

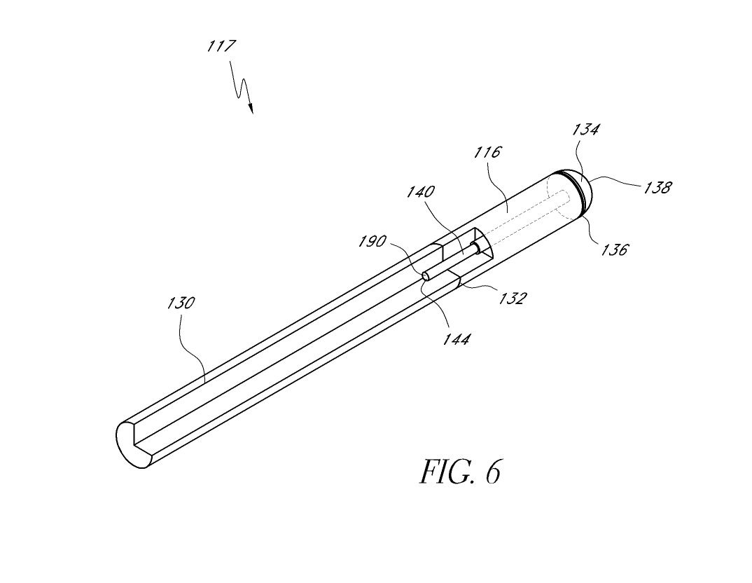

In certain embodiments, as illustrated in FIGS. 5 and 6, a reference material 190 may be attached to the proximal surface of the rod 144. The reference material 190 may be reflective (e.g., a mirror) and functions similar to embodiments in which the proximal surface of the rod 144 reflects at least a portion of the excitation light signal (or reference light signal) down the optical fiber 130 to a detector (not shown). In other embodiments, the reference material 190 comprises a separate dye indicator system, such as for example a glucose-insensitive fluorescent dye. The excitation light from the optical fiber 130 causes the glucose-insensitive fluorophore to emit a fluorescent light back to a detector (not shown) in order to reference the emission signal from the hydrogel/chemical indicator system. In certain embodiments, the separate dye indicator system is formed of a plastic material, such as for example polycarbonate, polyethylene, or polystyrene, infused with a fluorescent dye configured to emit a separate glucose-insensitive signal.

The hydrogel and glucose-sensing chemical indicator system is disposed within the cavity 116. In preferred embodiments, the hydrogel/chemical indicator system filled cavity 116 is covered by a selectively permeable membrane 142 that allows passage of glucose into and out of the hydrogel/chemical indicator system. In certain embodiments, the selectively permeable membrane is a microporous membrane. The microporous membrane may comprise one or more polymers selected from a group consisting of polyolefins, fluoropolymers, polycarbonates, and polysulfones. More preferably, the microporous membrane comprises at least one fluoropolymer. The at least one fluoropolymer may be selected from the group consisting of polytetrafluoroethylene, perfluoroalkoxy polymer, fluorinated ethylene-propylene, polyethylenetetrafluoroethylene, polyvinylfluoride, polyethylenechlorotrifluoroethylene, polyvinylidene fluoride, polychlorotrifluoroethylene, perfluoropolyether, perfluoroelastomer, and fluoroelastomer. In other embodiments of the analyte sensor, the microporous membrane comprises at least one polyolefin. The polyolefin is preferably polyethylene. Although these embodiments are described using a glucose sensor 117, it should be understood by a person of ordinary skill in the art that the sensor 117 can be modified to measure other analytes by changing, for example, the sensing chemistry, and if necessary, the selectively permeable membrane 142.

In certain embodiments, the selectively permeable membrane 142 is attached to the optical fiber 130 and the atraumatic tip 134 by means of an adhesive. In preferred embodiments, the adhesive is biocompatible, such as for example, cyanoacrylates, epoxies, light cure adhesives, silicones, and urethanes. In certain embodiments, after applying the adhesive and attaching the selectively permeable membrane 142 to the optical fiber 130 and atraumatic tip 134, the adhesive is cured at room temperature, by heating, or by applying UV/visible light. In certain embodiments, the time to adhere the selectively permeable membrane 142 to the optical fiber 130 and/or atraumatic tip 134 can vary from about 5 seconds to about 60 seconds, from about 15 minutes to about 5 hours, from about 60 seconds to about 10 minutes, or up to about 24 hours. In other embodiments, the selectively permeable membrane 142 is pre-fabricated as a sleeve. The sleeve may be slid into place and sealed using an adhesive and/or heated to form-fit the glucose sensor 117. In certain embodiments, the selectively permeable membrane 142 surrounds the entire circumference of the glucose sensor 117. In other embodiments, the selectively permeable membrane 142 covers a window 180 or opening in the glucose sensor 117 exposing the void or cavity 116 to analytes in the blood stream.

In some embodiments, as illustrated in FIGS. 2A and 2B, the sensor 117 comprises a distal portion and a proximal portion. The distal portion of the sensor 117 comprises the atraumatic tip portion 134, the hydrogel/chemical indicator system filled cavity 116, the rod 140, at least the portion of the selectively permeable membrane 142 that covers the cavity 116 and the distal end 132 of the optical fiber 130. The proximal portion of the sensor 117 comprises the proximal portion of the optical fiber 130. In some embodiments, the diameter, D1, of the distal portion of the sensor 117 is greater than the diameter, D2, of the proximal portion of the sensor 117. For example, the diameter D1 of the distal portion of the sensor 117 can be between about 0.0080 inches and 0.020 inches, while the diameter D2 of the proximal portion of the sensor 117 can be between about 0.005 inches to 0.015 inches. In some embodiments, the diameter D1 of the distal portion of the sensor 117 is about 0.012 inches, while the diameter D2 of the proximal portion of the sensor 117 is about 0.010 inches.

In some embodiments, the sensor 117, including the selectively permeable membrane 142, has a smooth surface. The smooth surface can be made for example by the method disclosed in US Patent Publication No. 2008/0187655. In summary, one preferred embodiment of the method comprises filling the cavity 116 with a solution comprising a monomer, crosslinker and an initiator, such as a thermal initiator. After the sensor 117 has been filled, the sensor 117 is dipped into liquid wax, which is allowed to harden around the sensor 117 and selectively permeable membrane 142.

The liquid wax has a melting point that is greater than the thermal initiation temperature. Therefore, in order to reduce the likelihood of initiation during the wax dipping and coating step, the filled sensor 117 can be chilled before the wax dipping and coating step. After the sensor 117 has been coated with wax, the solution in the cavity 116 can be deoxygenated by placing the sensor 117 in a water bath while bubbling an inert gas, such as nitrogen, in the bath.

After deoxygenation, polymerization can be initiated by heating the sensor 117 to a temperature above the thermal initiation temperature, but below the melting point of the wax. Once the solution is substantially polymerized into the hydrogel, the wax can be removed from the sensor by use of a solvent, such as hexane, leaving a sensor 117 with a smooth surface.

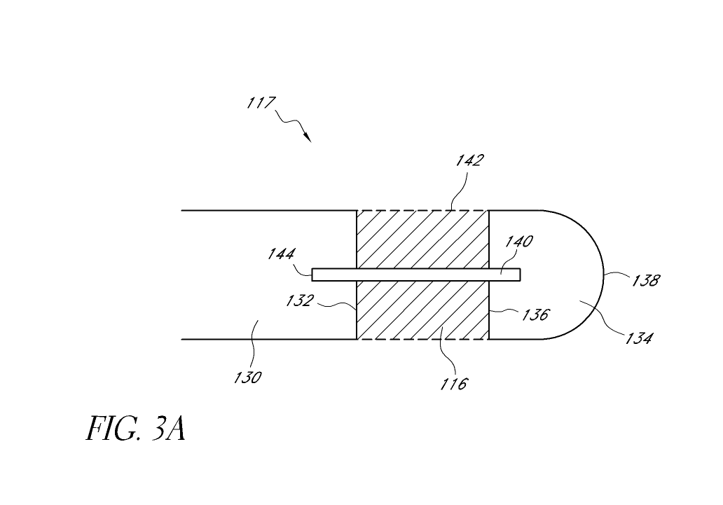

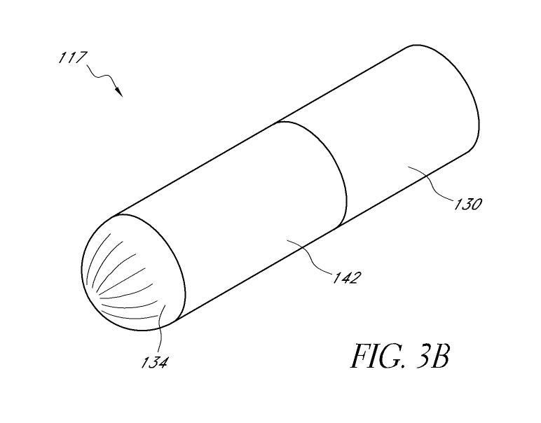

In some embodiments, as illustrated in FIGS. 3A and 3B, the sensor 117 comprises a distal portion and a proximal portion with substantially the same diameter. In some embodiments, the diameter of the sensor 117 is between about 0.005 inches and 0.020 inches. In other embodiments, the diameter of the sensor 117 is between about 0.008 inches and 0.014 inches. In other embodiments, the diameter of the sensor 117 is about 0.010 inches or about 0.012 inches.

In some embodiments, the rod 140 has a proximal portion that is connected to the distal portion of the optical fiber 130 and a distal portion that is connected to the proximal portion of the atraumatic tip portion 134. The rod 140 can be made of a metal, metal alloy, plastic, polymer, ceramic, composite material or any other material with suitable mechanical properties for connecting the atraumatic tip portion 134 with the distal portion of the optical fiber 130. For example, the rod 140 can be made of stainless steel, titanium or Nitinol. The rod 140 can be cylindrical or noncylindrical, such as a bar with a square, rectangular, oval or oblong cross-section. In some embodiments, the diameter of the rod 140 is generally between about 0.001 to 0.010 inches. In other embodiments, the diameter of the rod 140 is generally between about 0.004 to 0.008 inches. In other embodiments, the diameter of the rod 140 is about 0.001 inches, about 0.002 inches or 0.004 inches. In some embodiments, the diameter of the rod 140 may be less than about 0.001 inches. In some embodiments, the diameter of the rod 140 may be greater than about 0.010 inches. In some embodiments, the length of the rod 140 is generally less than about 0.005 inches. In some embodiments, the length of the rod 140 is between about 0.005 to 0.040 inches. In other embodiments, the length of the rod 140 is generally between about 0.020 to 0.040 inches. In other embodiments, the length of the rod 140 is generally about 0.015 inches. In some embodiments, the length of the rod 140 is generally greater than about 0.005 inches.

The rod 140 adds mechanical stability to the distal portion of the sensor 117. In some embodiments, the rod 140 also adds flexibility to the distal portion of the sensor 117, allowing the atraumatic tip portion 134 to flex back and forth relative to the orientation of the optical fiber 130. The flexibility of the rod 140, and thus the degree which the atraumatic tip portion 134 can flex, can be increased or decreased by decreasing or increasing the diameter of the rod 140. In addition, the flexibility of the rod 140 can be altered by making the rod 140 from a stiff or flexible material.

In some embodiments as illustrated in FIGS. 2A and 3A, a reflective surface 144 is disposed on the proximal end of the rod 140, which is inserted into the optical fiber 130. The reflective surface 144 is capable of reflecting back at least a portion of either reference light or excitation light emitted from the light source. The other end of the rod 140 is inserted into the atraumatic tip portion 134. In certain embodiments, the atraumatic tip portion may be made from a non-reflective material, for example polyethylene (e.g., black polyethylene) or polypropylene. The reference or excitation light that passes though the optical fiber 130 in the region corresponding to the diameter of the rod 140 is reflected off the reflective surface 144 without entering into the hydrogel filled cavity 116; the amount of light entering the hydrogel/chemical indicator system can be controlled by varying the diameter/cross-sectional area of the rod and/or by attaching a mirror or other reflective member 190 (illustrated in FIGS. 5 and 6) having a selected cross-sectional area to the proximal end of the rod. The hydrogel filled cavity 116 is preferably covered by a selectively permeable membrane 142, which is at least permeable to glucose. Therefore, the reflected reference or excitation light and the ratio between the reflected and emitted light is independent of the temperature, pH, glucose concentration, and chemistry formulation of the hydrogel filled cavity 116. The ratio between the reflected and emitted light is dependent, however, on the diameter of the rod and the ratio of the diameter of the rod to the area of the sensor. In certain embodiments, the rod 140 is sufficiently stiff to keep the hydrogel filled cavity 116 in a fixed orientation relative to the optical fiber 130 so that any light that is transmitted through the hydrogel cavity 116 is not reflected back to the optical fiber 130.

With regard to FIG. 3B, there is shown a perspective view of the distal region of the sensor 117 illustrated in FIG. 3A. It can be appreciated in the illustrated embodiment that there is no change in sensor diameter from the optical fiber 130, through the membrane 142 covered hydrogel cavity, until the tapered atraumatic distal tip portion 134.

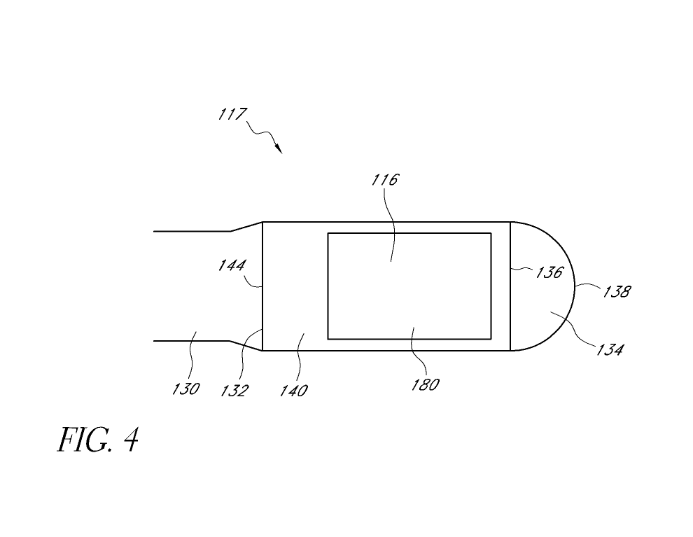

In some embodiments, as shown in FIG. 4, a window 180 is cut into a hypotube 140. The distal end 132 of the optical fiber 130 is inserted onto the reflective surface 144 (e.g., an annular mirror) and then heated such that the optical fiber 130 swells to fully contact the reflective surface 144 of the hypotube 140. In certain embodiments, heating the optical fiber 130 is carried out in a glass tube. In other embodiments, heating the optical fiber 130 is carried out in an oven. In still other embodiments, the optical fiber 130 is attached to the hypotube 140 using an adhesive. In preferred embodiments, the adhesive is biocompatible, such as for example, cyanoacrylates, epoxies, light cure adhesives, silicones, and urethanes. In certain embodiments, after applying the adhesive and attaching the optical fiber 130 to the hypotube 140, the adhesive is cured at room temperature, by heating, or by applying UV/visible light. In certain embodiments, the time to adhere the optical fiber 130 to the hypotube 140 can vary from about 5 seconds to about 60 seconds, from about 15 minutes to about 5 hours, from about 60 seconds to about 10 minutes, or up to about 24 hours. Similar methods may be employed for attaching the hypotube 140 to the atraumatic tip 134. Similar to previous embodiments, the reference or excitation light is reflected off the reflective surface 144 without entering the window 180 that opens to the cavity 116 which is filled with hydrogel/chemical indicator system. Therefore, the reflected reference or excitation light and the ratio between the reflected and emitted lights is independent of the temperature, pH, glucose concentration, and hydrogel chemistry. The surface area of the reflective surface can be varied to control the amount of excitation light that enters the hydrogel/chemical indicator system filed cavity 116. The distal end 136 of the hypotube 140, as in previous embodiments, may have a non-reflective surface, such as a black plug made of polyethylene so that light entering the hydrogel/chemical indicator system filled cavity 116 is not reflected back into the optical fiber 130. In some embodiments, the window 180 containing the hydrogel/chemical indicator system filled cavity 116 is covered by a selectively permeable membrane (not shown).

In some embodiments, as illustrated in FIG. 5, the glucose sensor 117 includes a cage 195, as an outer shell, connecting the atraumatic tip 134 with the optical fiber 130. The cage 195 can add mechanical stability to the distal portion of the sensor 117. In some embodiments, the cage 195 also adds flexibility to the distal portion of the sensor 117, allowing the atraumatic tip portion 134 to flex back and forth relative to the orientation of the optical fiber 130. The flexibility of the cage 195, and thus the degree which the atraumatic tip portion 134 can flex, can be increased or decreased by decreasing or increasing the thickness of the cage 195 walls. In addition, the flexibility of the cage 195 can be altered by making the cage 195 from a stiff or flexible material. In certain embodiments, the thickness of the cage 195 walls is about 0.001 inches, about 0.002 inches, or about 0.004 inches. In some embodiments, the thickness of the cage 195 walls may be less than about 0.001 inches. In some embodiments, the thickness of the cage 195 walls may be greater than about 0.010 inches.

In some embodiments, the diameter of the optical fiber 130 may be smaller than the diameter of the interior of the cage 195, allowing the optical fiber 130 to fit within the interior of the cage 195 and abut the void or cavity 116. For example, the diameter of the optical fiber 130 may be between about 0.005 inches and about 0.020 inches, between about 0.008 inches and about 0.014 inches, or between about 0.010 inches and about 0.012 inches. The diameter of the interior of the cage 195 may be about 0.001 inches larger.

In certain embodiments, the cage 195 has a window or opening 180, covered by a selectively permeable membrane 142 (not shown), which allows for at least the transmission of analytes, such a glucose, into the void or cavity 116. In certain preferred embodiments, the void or cavity 116 is filled with a hydrogel/chemical indicator system. A rod 140 may be positioned within the glucose sensor 117 having a reference material 190. As discussed above, the reference material 190 may be a mirror for reflecting excitation light from the optical fiber 130 back to a detector (not shown) or a glucose-insensitive fluorescent dye for emitting a glucose-insensitive reference signal back to a detector (not shown). The combination of the cage 195 and the rod 140 may provide a sufficiently rigid structure such that the excitation light which enters the void or cavity 116 remains separate from the light that enters the reference material 190.

In some embodiments, as illustrated in FIG. 6, the glucose sensor 117 does not have a cage 195 surrounding the void or cavity 116. Instead, similar to FIGS. 2A and 3A, the rod 140 connects the optical fiber 130 and atraumatic tip 134, providing structure for the glucose sensor 117, and is surrounded by the void or cavity 116, which in turn is covered by a selectively permeable membrane 142. Similar to FIG. 3A, the diameter of the optical fiber 130 is the same as the diameter of the hydrogel/chemical indicator system encased cavity 116. As discussed with respect to FIG. 5, the rod may have a reference material 190 attached to the proximal surface of the rod 144, which functions as previously discussed.

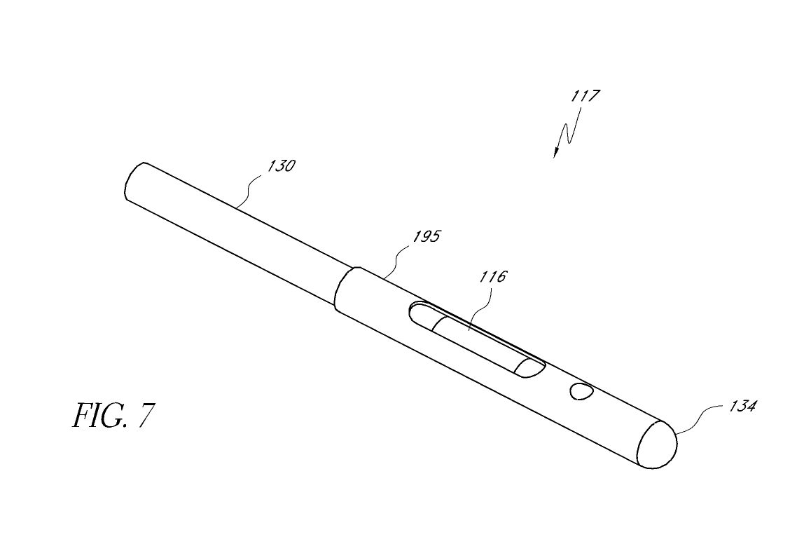

FIGS. 7-11 illustrate certain embodiments having different configurations for the reference material 190. As discussed previously, the reference material 190 in each of these embodiments may either comprise reflective material to return at least a portion of the excitation light back to a detector (not shown) or a separate dye indicator system to return an emission signal back to a detector (not shown). Similar to previous embodiments, the excitation or reference light is reflected off the reflective surface 190 without entering the cavity 116, which is filled with the hydrogel/chemical indicator system. Likewise, the emitted or reference light from the separate dye indicator system is independent of the glucose concentration. Therefore, the reference light and the ratio between the reference light and emitted glucose concentration dependent lights are independent of the temperature, pH, glucose concentration, and hydrogel chemistry. The surface area, shape, and configuration of the reference material 190 can be varied to control the amount of excitation light that enters the hydrogel/chemical indicator system filed cavity 116. The distal end 136 of the rod or hypotube 140, as in previous embodiments, or reference material 190 may have a non-reflective surface, such as a black plug made of polyethylene so that light entering the hydrogel/chemical indicator system filled cavity 116 is not reflected back into the optical fiber 130.

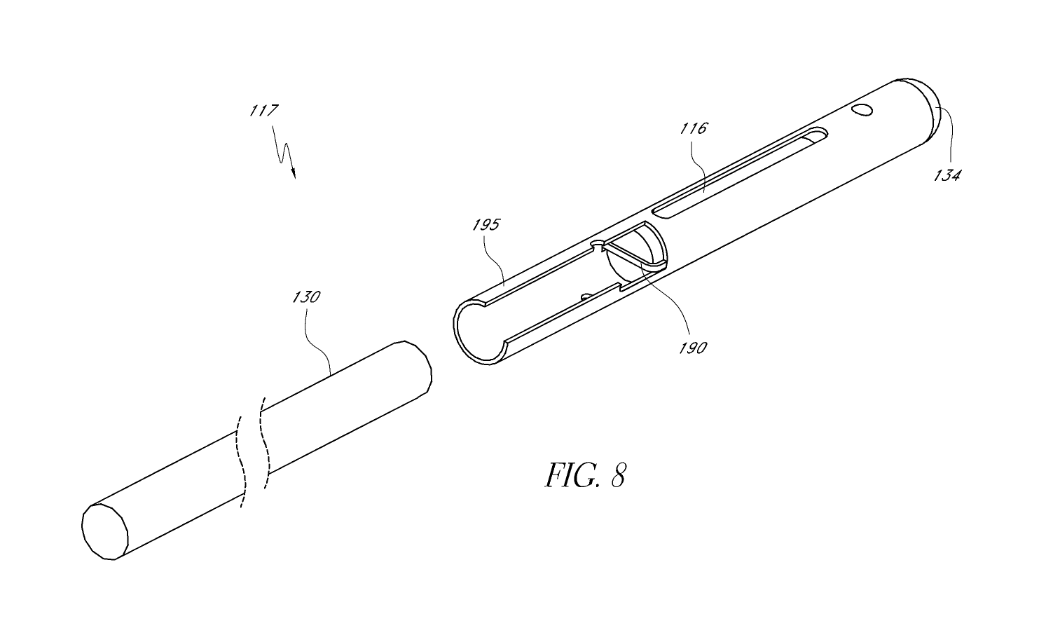

In FIG. 7, the reference material 190 abuts the void or cavity 116 beneath the cage 195 and extends to and comprises the atraumatic tip 134. In certain embodiments, the atraumatic tip 134 is formed of a glucose-insensitive red dye plastic material. In FIG. 8, the reference material 190 is a reflective strip that spans the diameter of the hydrogel-filled cavity 116. The term reflective strip is used herein to refer to any elongate member, regardless of its geometry, width, or thickness that spans at least a cross-section of the glucose sensor 117. The reflective strip 190 may be centered at the diameter of the glucose sensor 117 (as illustrated) or off-centered with regard to the cross-section of the cage 195 or optical fiber 130. In some embodiments, there may be more than one reflective strip in one or more locations within the glucose sensor 117. Where more than one reflective strip is employed, the reflective strip may be arranged symmetrically or asymmetrically with respect to the cross-section of the glucose sensor 117. In certain embodiments, the reflective strip 190 may be between about 0.001 inches and about 0.005 inches wide and between about 0.001 inches and about 0.005 inches thick.

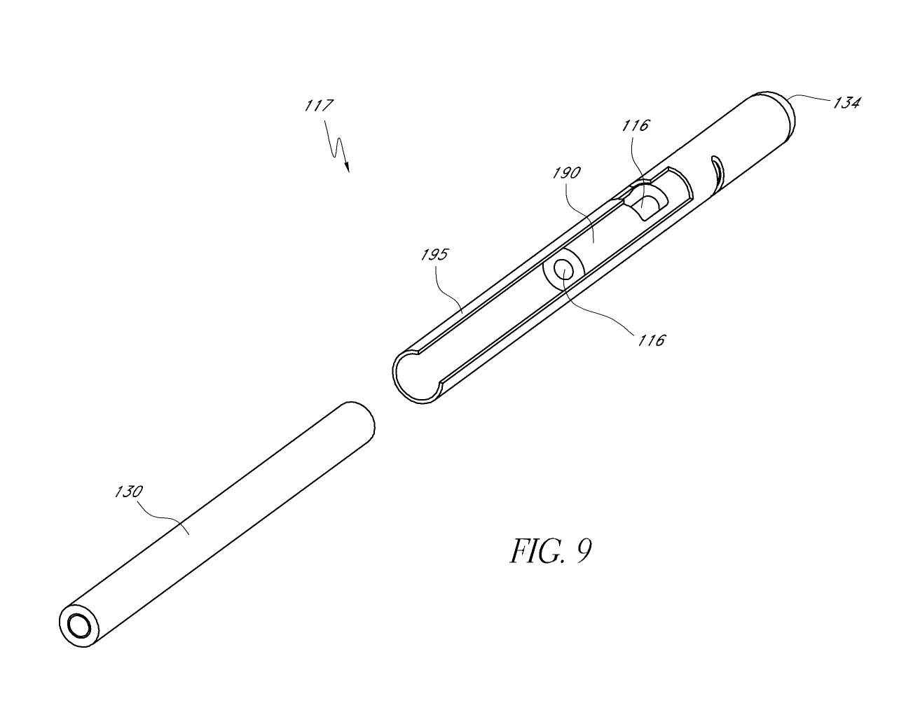

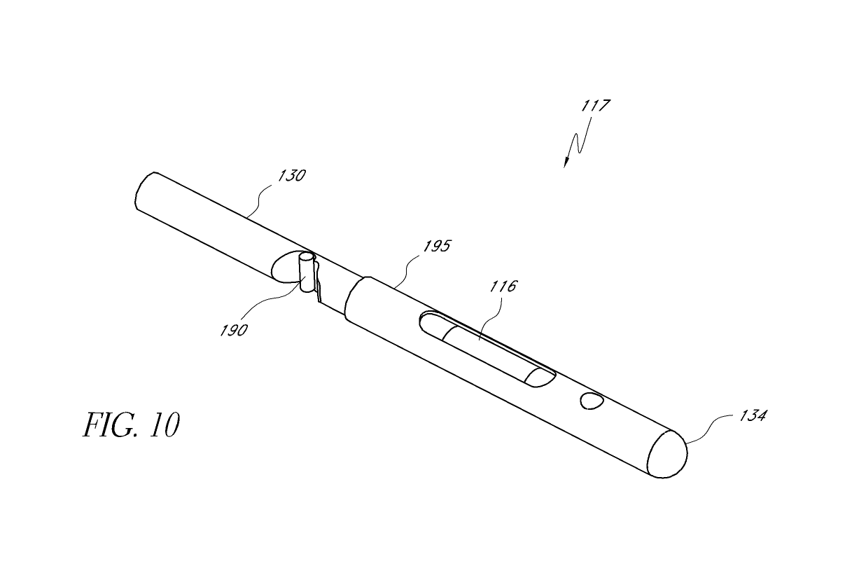

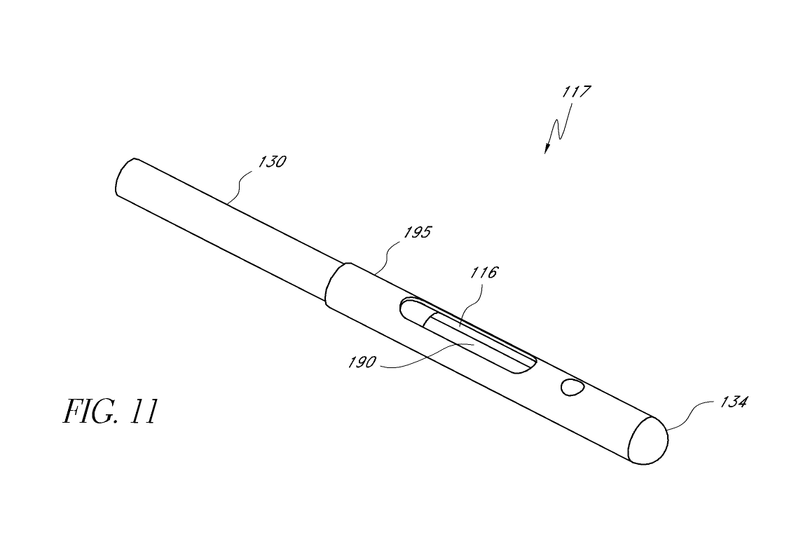

In FIG. 9, the reference material 190 is disposed within the cage 195 as a hypotube containing the hydrogel-filled cavity 116 and having a reflective surface or annular mirror at the proximal surface 144 of the hypotube 140. In certain embodiments, the hypotube 140 has an outer diameter equal to the outer diameter of the optical fiber 130. In FIG. 10, similar to FIG. 8, the reference material 190 is reflective strip, but the reflective strip in FIG. 10 is placed within a hole drilled in the optical fiber 130, rather than abutting the hydrogel-filled cavity 116. FIG. 11 illustrates an embodiment in which the reference material 190 is located in the cavity 116 and is in a side-by-side configuration with the hydrogel/chemical indicator system.

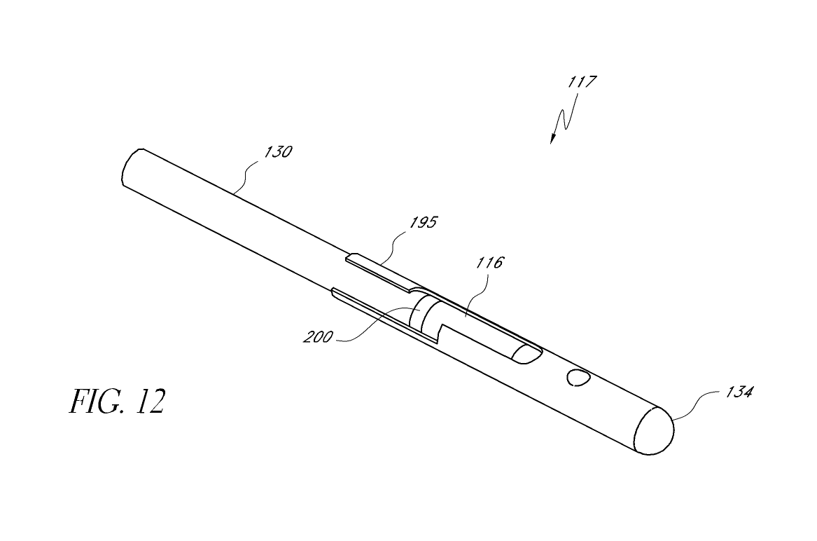

In certain embodiments, as illustrated in FIG. 12, a reference material 200 comprises a translucent material. In certain embodiments, this translucent material comprises a red dye, such as the glucose-insensitive fluorescent dye discussed previously. The red dye may allow some of the excitation light to be transmitted to the hydrogel-filled cavity 116, may reflect some of the excitation light back to a detector (not shown) before the excitation light reaches the hydrogel-filled cavity 116, and may emit a separate glucose-insensitive signal to a detector (not shown).

A person skilled in the art would readily understand that the above described embodiments, or components of the above described embodiments, may be combined within the scope of the present invention. For example, a glucose sensor may contain one or more structural elements, such as a cage, a hypotube, and/or a rod within the scope of the present invention. In addition, a glucose sensor may contain one or more reference materials, functioning as a reflective surface and/or as a separate dye indicator system, in different locations and configurations within the scope of the present invention.

In some embodiments (see e.g., FIGS. 2-12), the glucose sensor 117 comprises an atraumatic tip portion 134. The atraumatic tip portion 134 has a distal end 138 that is curved and substantially free of sharp edges. In addition, the atraumatic tip portion 134 can be flexible and deformable. The distal end 138 of the atraumatic tip portion 134 can be hemispherical, parabolic, elliptical or curved in any other suitable shape that is reduces the risk of injury to the patient. The atraumatic tip portion 134 can be made from a variety of materials, such as plastics, polymers, gels, metals and composites of the above.

In some embodiments, the glucose sensor 117 includes a temperature sensor or probe 146, such as thermocouple or thermistor (See e.g., FIG. 2A). The temperature sensor 146 can measure the temperature of the hydrogel and glucose sensing chemistry system, and/or the blood when disposed intravascularly. The temperature sensor 146 is particularly preferred when the glucose-sensing chemistry is affected by temperature. For example, in some embodiments, the fluorescence intensity emitted by the fluorophore system is dependent on the temperature of the fluorophore system. By measuring the temperature of the fluorophore system, temperature induced variations in fluorophore fluorescence intensity can be accounted for, allowing for more accurate determination of glucose concentration.

In certain embodiments, the temperature sensor can be a thermistor (as described above with regard to FIG. 2A, reference numeral 146, a platinum resistance temperature device (“RTD”), another RTD, a thermocouple, an infrared-based temperature detector, a fluorescence-based temperature sensing element, or other temperature sensing elements with determinable temperature-dependent characteristics.

Devices such as thermistors, platinum RTDs, and other RTDs generally require one or more conductors, such as wires, to conduct the output of the sensor to a receiving unit which converts the output to a temperature signal. The conductors can be bundled with the optical fiber of fluorescence-based glucose sensors, such as those discussed above, or they can be routed separately. In one embodiment, the temperature sensor is placed inside the body, and the receiver is placed outside the body. In another embodiment, the temperature sensor is placed inside the body, and a transmitter, signal processor, etc. is also placed inside the body and is connected to or is a part of the temperature sensor. In preferred embodiments, the temperature sensing element is located at or near the glucose sensing moiety.

In another embodiment, a fluorescence-based temperature sensing technique can be used. Fluorescence-based temperature sensing techniques include those based on fluorescence decay, such as where an excitation light is provided to a phosphor, the excitation light is stopped, and the fluorescence is monitored versus time, with the rate of decrease in fluorescence being related to the temperature of the phosphor. Various techniques, can also include phase measurement and phase angle analysis.

Methods for performing fluorescence-based temperature measurement have been described. See for example, LumaSense Technologies, Inc. (Santa Clara, Calif.), LumaSMART and ThermaAsset2. Fluorescent materials that can be used in fluorescence-based temperature measurement are known to, or readily identified by those having skill in the art.

In some embodiments, the fluorescent material can be surrounded by material which prevents or inhibits chemical interaction between the fluorescent material and blood components. Suitable materials include glass (for example, borosilicate, lime-soda, or other types including those used for fiberoptic cables), polymers (for example, Teflon, fluoropolymers, silicone, latex, polyolefins, polyisoprene, and other rigid and nonrigid polymeric materials), metals (for example, 300 series stainless steel, 400 series stainless steel, nickel, nickel alloys, chromium steels, zirconium and its alloys, titanium and its alloys, as well as other corrosion resistant metals and alloys including exotic metals and alloys), ceramics (for example, ceramic materials related to aluminum oxide, silica and oxide, zirconium, carbides, etc.), and combinations of these.

In some embodiments, the temperature sensor can be positioned within the glucose sensor, or near it. While in one preferred embodiment, the temperature sensor can be positioned as close as possible to (e.g., within) the glucose-sensing chemical indicator system of the glucose sensor, positions some distance away can also be successfully utilized, including those locations where the temperature measured provides an indication of the temperature at the glucose-sensing site(s) within an acceptable error for the use for which the temperature measurement is being made.

In some embodiments, the temperature sensor and/or the leads to the sensor can be isolated from the physiological environment, such as by coating, covering, or encasing the various parts with a material that prevents or inhibits chemical or physical interaction between the temperature sensor and/or its leads and blood components. Chemical interactions that are preferably avoided include corrosion, leaching of chemical species, generation of additional signals (e.g. optical, electrical, etc.) and take-up by the body of materials present in the sensor or leads, whether present from manufacture, corrosion or other means, such as compounds, metals, or ions causing a physiological response in some patients including copper, silver, organic compounds, organometallic compounds, etc.

Physical interactions can include breakage and physical separation (e.g. disconnection and potential loss), signal leakage (e.g. optical; electrical, etc.), signal degradation (including resistance, stray signal detection, noise, capacitance, electrochemical effects, induced voltages, ground loops, etc.). Suitable materials include glass (e.g., borosilicate, lime-soda, as well as other types of glass, such as those used in production of optic fibers), polymers (e.g., Teflon, fluoropolymers, silicone, latex, polyolefins, polyisoprene, acrylics, polycarbonates, and other rigid and nonrigid polymeric materials), metals (e.g., 300 series stainless steel, 400 series stainless steel, nickel, nickel alloys, chromium steels, zirconium and its alloys, titanium and its alloys, as well as other corrosion resistant metals and alloys including exotic metals and alloys), ceramics (e.g., ceramic materials related to aluminum oxide, silica and oxide, zirconium, carbides, etc.), and combinations of these.

Suitable methods for applying for isolating material to the temperature sensor or leads can include any appropriate method, including casting, painting, dipping, gluing, reacting, drawing, depositing, mechanically adhering, encapsulating, etc.

In some embodiments, suitable sizes for temperature sensors that will be incorporated into the glucose sensor include those temperature sensing elements resulting in an overall glucose sensor of between about 0.005 inches and 0.020 inches.

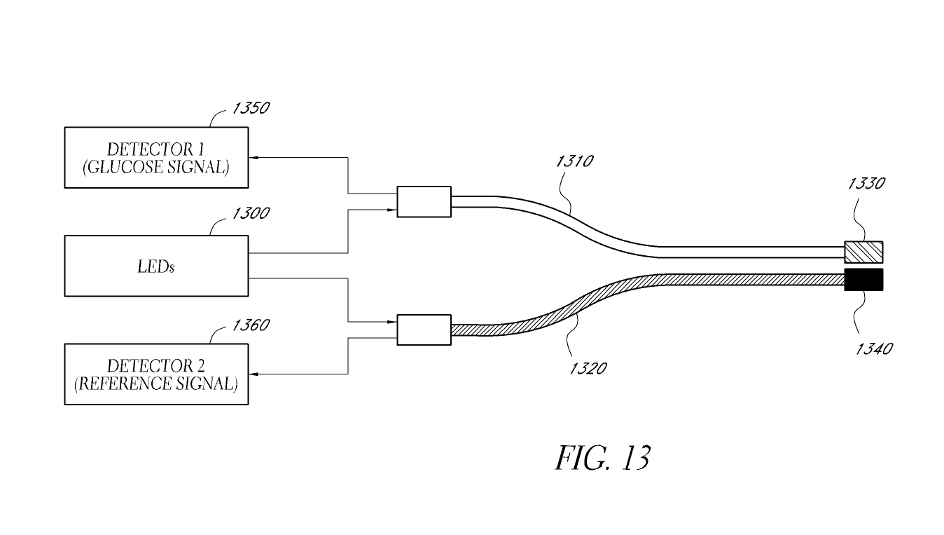

FIG. 13 illustrates another embodiment for measuring the glucose concentration in comparison to a reference signal. In this embodiment, a LED source 1300 sends an excitation signal down two separate adjacent optical fibers 1310, 1320. The first optical fiber, or the glucose fiber 1310, has a proximal tip and a distal tip. The distal tip has a glucose sensing hydrogel 1330 which contains a fluorophore or dye, a quencher, and glucose binding receptors. The second optical fiber, or the reference fiber 1320, also has a proximal tip and a distal tip. The distal tip of the reference fiber has a reference material 1340. In certain embodiments, the reference material 1340 contains the same or a different fluorophore or dye, may or may not contain the quencher, but does not contain glucose receptors. In other embodiments, the reference material 1340 has the same exact glucose sensing hydrogel, but it is encased in a glucose impermeable membrane. In both of these embodiments, the reference fiber 1320 emits a fluorescent return signal independent of the glucose concentration.

After the excitation light passes through the glucose fiber 1310 and the reference fiber 1320, the glucose sensing hydrogel 1330 and the reference material 1340 emit fluorescent signals back to two separate detectors, a glucose signal detector 1350 and a reference signal detector 1360, for ratiometric processing. The benefit of the dual fiber configuration is that both fibers 1310, 1320 experience the same external pressure, bending, temperature, and other external factors. In addition, both fibers 1310, 1320 contain substantially the same material in the glucose sensing hydrogel 1330 and reference material 1340. As a result, the ratio of the intensities between the two fibers 1310, 1320, as measured by the detectors 1350, 1360, produce a calibrated glucose signal that removes, inter alia, the effect of the fluctuations in the LED output or altered transmission along the optical fiber, and thereby increase the accuracy in the measurement of the glucose concentration.

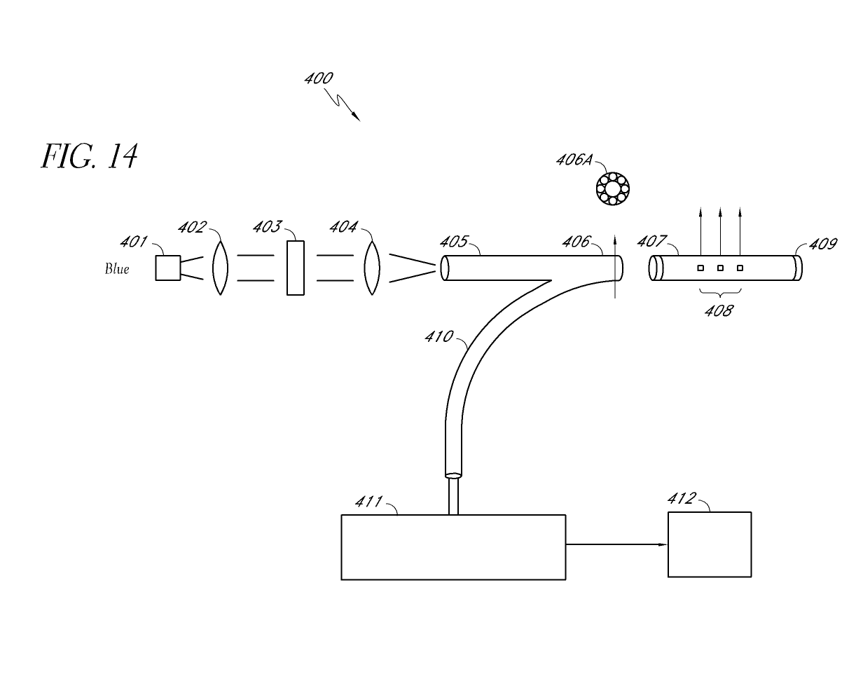

With reference to FIG. 14, in certain embodiments, the light generated by the single light source 401 is transmitted through a optical module comprising a collimator lens 402, an interference filter 403, and/or a focusing lens 404 as described above. The resulting light can be filtered through an interference filter 403. The resulting light can be focused by a focusing lens 404 into an optical fiber 405, which may be a single fiber or a bundle of fibers. The optical fiber 405 can surround optical fiber 410 as both fiber optic lines connect to the first end of the glucose sensor 407. In certain embodiments, a mirror or reflective surface 409 is attached to the second end of the glucose sensor 407. The optical fiber 410 may be a single fiber or a bundle of fibers. The glucose sensor can comprise hydrogels that further comprise a fluorophore system that produces two emission wavelengths, a first emission wavelength and a second emission wavelength. In certain embodiments, the fluorophore system is excited by the light generated by light source 401. In certain embodiments, the optical fiber 410 is connected to a light sensitive module comprising a microspectrometer 411 that measures the entire spectrum of light in the glucose measurement system 400. Data from the microspectrometer 411 can be transmitted to computer 412 for processing. The microspectrometer 411 can allow system 400 to simultaneously measure the excitation light intensity as well as both emission light intensities. Ratiometric calculations may be employed to substantially eliminate or reduce non-glucose related factors affecting the intensity of the measured emission light and measured excitation light (as detailed in US Patent Publication No. 2008/0188725; incorporated herein in its entirety by reference thereto). The measured emission light can be divided by the measured excitation light, wherein such calculations substantially eliminate or reduce non-glucose related factors affecting the intensity of the lights.

In certain preferred embodiments, the fluorophore dye may be selected such that it exists in distinguishable acid and base conformations, each of which emit at a distinct wavelength, and wherein the relative proportion of acid and base forms depend on the pH. The ratio of intensities of the acid and base emissions can be used to determine the pH of the blood (as detailed in U.S. Pat. No. 7,751,863; incorporated herein in its entirety by reference thereto). The ratio of the acid or base emission intensity over the excitation light can be used to determine the level of glucose in the blood. Of course in a variation to this single exciter-dual emitter fluorophore system, one could employ a single exciter-single emitter for detection of glucose concentration without simultaneous ratiometric determination of pH. Indeed, a great variety of design options are available (see e.g., U.S. Pat. No. 7,751,863, US Patent Publication No. 2008/0188725, and U.S. Provisional patent application Ser. No. 13/095,748), wherein the chemical indicator and optical systems may be selected based on the preferred use.

### Glucose-Sensing Chemical Indicator Systems

In certain embodiments, the hydrogels are associated with a plurality of fluorophore systems. In certain embodiments, the fluorophore systems comprise a quencher with a glucose receptor site. In certain embodiments, when there is no glucose present to bind with the glucose receptor, the quencher prevents the fluorophore system from emitting light when the dye is excited by an excitation light. In certain embodiments, when there is glucose present to bind with the glucose receptor, the quencher allows the fluorophore system to emit light when the dye is excited by an excitation light.

In certain embodiments, the emission produced by the fluorophore system may vary with the pH (as well as the temperature) of the solution (for example, blood), such that different excitation wavelengths (one exciting the acid form of the fluorophore and the other the base form of the fluorophore) produce different emissions signals. In preferred embodiments, the ratio of the emission signal from the acid form of the fluorophore over the emission signal from the base form of the fluorophore is related to the pH level of the blood. In certain embodiments, an interference filter is employed to ensure that the two excitation lights are exciting only one form (the acid form or the base form) of the fluorophore. Chemical indicator systems, hardware configurations and methods for determining both pH and glucose based on ratiometric determination are described in detail in U.S. Pat. No. 7,751,863 and U.S. Patent Publication No. 2008/0188725, incorporated herein in their entirety by reference thereto.

The indicator system (also referred to herein as a fluorophore system) can comprise a fluorophore operably coupled to a quencher. In certain embodiments, the fluorophore system comprises a polymer matrix comprising a fluorophore susceptible to quenching by a viologen, a viologen quencher with quenching efficacy dependent on glucose concentration, and a glucose permeable polymer, wherein said matrix is in contact with blood in vivo. Preferably the fluorophore is a fluorescent organic dye, the quencher is a boronic acid functionalized viologen, and the matrix is a hydrogel.

“Fluorophore” refers to a substance that when illuminated by light at a particular wavelength emits light at a longer wavelength; i.e. it fluoresces. Fluorophores include but are not limited to organic dyes, organometallic compounds, metal chelates, fluorescent conjugated polymers, quantum dots or nanoparticles and combinations of the above. Fluorophores may be discrete moieties or substituents attached to a polymer.

Fluorophores that may be used in preferred embodiments are capable of being excited by light of wavelength at or greater than about 400 nm, with a Stokes shift large enough that the excitation and emission wavelengths are separable by at least 10 nm. In some embodiments, the separation between the excitation and emission wavelengths may be equal to or greater than about 30 nm. These fluorophores are preferably susceptible to quenching by electron acceptor molecules, such as viologens, and are resistant to photo-bleaching. They are also preferably stable against photo-oxidation, hydrolysis and biodegradation.

In some embodiments, the fluorophore may be a discrete compound.

In some embodiments, the fluorophore may be a pendant group or a chain unit in a water-soluble or water-dispersible polymer having molecular weight of about 10,000 daltons or greater, forming a dye-polymer unit. In one embodiment, such dye-polymer unit may also be non-covalently associated with a water-insoluble polymer matrix M¹ and is physically immobilized within the polymer matrix M¹, wherein M¹ is permeable to or in contact with an analyte solution. In another embodiment, the dye on the dye-polymer unit may be negatively charged, and the dye-polymer unit may be immobilized as a complex with a cationic water-soluble polymer, wherein said complex is permeable to or in contact with the analyte solution. In one embodiment, the dye may be one of the polymeric derivatives of hydroxypyrene trisulfonic acid. The polymeric dyes may be water-soluble, water-swellable or dispersible in water. In some embodiments, the polymeric dyes may also be cross-linked. In preferred embodiments, the dye has a negative charge.

In other embodiments, the dye molecule may be covalently bonded to the water-insoluble polymer matrix M¹, wherein said M¹ is permeable to or in contact with the analyte solution. The dye molecule bonded to M¹ may form a structure M¹-L¹-Dye. L¹ is a hydrolytically stable covalent linker that covalently connects the sensing moiety to the polymer or matrix. Examples of L¹ include lower alkylene (e.g., C₁-C₈ alkylene), optionally terminated with or interrupted by one or more divalent connecting groups selected from sulfonamide (—SO₂NH—), amide —(C═O)N—, ester —(C═O)—O—, ether —O—, sulfide —S—, sulfone (—SO₂—), phenylene —C₆H₄—, urethane —NH(C═O)—O—, urea —NH(C═O)NH—, thiourea —NH(C═S)—NH—, amide —(C═O)NH—, amine —NR— (where R is defined as alkyl having 1 to 6 carbon atoms) and the like, or a combination thereof. In one embodiment, the dye is bonded to a polymer matrix through the sulfonamide functional groups.

In one preferred embodiment, the fluorophore may be HPTS-CysMA (structure illustrated below); see U.S. Pat. No. 7,417,164, incorporated in its entirety herein by reference thereto.

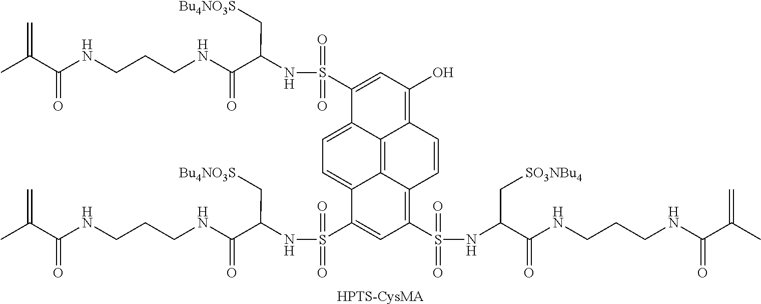

Of course, in some embodiments, substitutions other than Cys-MA on the HPTS core are consistent with aspects of the present invention, as long as the substitutions are negatively charged and have a polymerizable group. Either L or D stereoisomers of cysteine may be used. In some embodiments, only one or two of the sulfonic acids may be substituted. Likewise, in variations to HPTS-CysMA shown above, other counterions besides NBu₄⁺ may be used, including positively charged metals, e.g., Na⁺. In other variations, the sulfonic acid groups may be replaced with e.g., phosphoric, carboxylic, etc. functional groups.

Fluorescent dyes, including HPTS and its derivatives are known and many have been used in analyte detection. See e.g., U.S. Pat. Nos. 6,653,141, 6,627,177, 5,512,246, 5,137,833, 6,800,451, 6,794,195, 6,804,544, 6,002,954, 6,319,540, 6,766,183, 5,503,770, and 5,763,238; each of which is incorporated herein in its entirety by reference thereto.

In accordance with broad aspects of the present invention, the analyte binding moiety provides the at least dual functionality of being able to bind analyte and being able to modulate the apparent concentration of the fluorophore (e.g., detected as a change in emission signal intensity) in a manner related to the amount of analyte binding. In preferred embodiments, the analyte binding moiety is associated with a quencher. “Quencher” refers to a compound that reduces the emission of a fluorophore when in its presence. Quencher (Q) is selected from a discrete compound, a reactive intermediate which is convertible to a second discrete compound or to a polymerizable compound or Q is a pendant group or chain unit in a polymer prepared from said reactive intermediate or polymerizable compound, which polymer is water-soluble or dispersible or is an insoluble polymer, said polymer is optionally crosslinked.

In one example, the moiety that provides glucose recognition in the embodiments is an aromatic boronic acid. The boronic acid is covalently bonded to a conjugated nitrogen-containing heterocyclic aromatic bis-onium structure (e.g., a viologen). “Viologen” refers generally to compounds having the basic structure of a nitrogen containing conjugated N-substituted heterocyclic aromatic bis-onium salt, such as 2,2′-, 3,3′- or 4,4′-N,N′ bis-(benzyl)bipyridium dihalide (i.e., dichloride, bromide chloride), etc. Viologen also includes the substituted phenanthroline compounds. The boronic acid substituted quencher preferably has a pKa of between about 4 and 9, and reacts reversibly with glucose in aqueous media at a pH from about 6.8 to 7.8 to form boronate esters. The extent of reaction is related to glucose concentration in the medium. Formation of a boronate ester diminishes quenching of the fluorophore by the viologen resulting in an increase in fluorescence dependent on glucose concentration. A useful bis-onium salt is compatible with the analyte solution and capable of producing a detectable change in the fluorescent emission of the dye in the presence of the analyte to be detected.

Bis-onium salts in the embodiments of this invention are prepared from conjugated heterocyclic aromatic di-nitrogen compounds. The conjugated heterocyclic aromatic di-nitrogen compounds are selected from dipyridyls, dipyridyl ethylenes, dipyridyl phenylenes, phenanthrolines, and diazafluorenes, wherein the nitrogen atoms are in a different aromatic ring and are able to form an onium salt. It is understood that all isomers of said conjugated heterocyclic aromatic di-nitrogen compounds in which both nitrogens can be substituted are useful in this invention. In one embodiment, the quencher may be one of the bis-onium salts derived from 3,3′-dipyridyl, 4,4′-dipyridyl and 4,7-phenanthroline.

In some embodiments, the viologen-boronic acid adduct may be a discrete compound having a molecular weight of about 400 daltons or greater. In other embodiments, it may also be a pendant group or a chain unit of a water-soluble or water-dispersible polymer with a molecular weight greater than about 10,000 daltons. In one embodiment, the quencher-polymer unit may be non-covalently associated with a polymer matrix and is physically immobilized therein. In yet another embodiment, the quencher-polymer unit may be immobilized as a complex with a negatively charge water-soluble polymer.

In other embodiments, the viologen-boronic acid moiety may be a pendant group or a chain unit in a crosslinked, hydrophilic polymer or hydrogel sufficiently permeable to the analyte (e.g., glucose) to allow equilibrium to be established.

In other embodiments, the quencher may be covalently bonded to a second water-insoluble polymer matrix M², which can be represented by the structure M²-L²-Q. L² is a linker selected from the group consisting of a lower alkylene (e.g., C₁-C₈ alkylene), sulfonamide, amide, quaternary ammonium, pyridinium, ester, ether, sulfide, sulfone, phenylene, urea, thiourea, urethane, amine, and a combination thereof. The quencher may be linked to M² at one or two sites in some embodiments.

In certain embodiments, at least one quencher precursor is used to attach the quenching moiety to at least one polymer. For example, aromatic groups may be used to functionalize a viologen with combinations of boronic acid groups and reactive groups. In certain embodiments, this process includes attaching an aromatic group to each of the two nitrogens in the dipyridyl core of the viologen. At least one boronic acid group, a reactive group, or a combination of the two are then attached to each aromatic group, such that the groups attached to each of the two nitrogens on the dipyridyl core of the viologen may either be the same or different. Certain combinations of the functionalized viologen quenching moiety are described as follows:

a) a first aromatic group having a pendent reactive group is attached to the first nitrogen and a second aromatic group having at least one pendent boronic group is attached to the second nitrogen;

b) one or more boronic acid groups are attached to a first aromatic group, which is attached to the first nitrogen, and one boronic acid group and a reactive group are attached to a second aromatic group, which second aromatic group is attached to the second nitrogen;

c) one boronic acid group and a reactive group are attached to a first aromatic group, which first aromatic group is attached to the first nitrogen, and one boronic acid group and a reactive group are attached to a second aromatic group, which is attached to the second nitrogen; and

d) one boronic acid group is attached to an aromatic group, which aromatic group is attached to each of the two nitrogens, and a reactive group is attached to a carbon in a heteroaromatic ring in the heteroaromatic centrally located group.

Preferred embodiments comprise two boronic acid moieties and one polymerizable group or coupling group wherein the aromatic group is a benzyl substituent bonded to the nitrogen and the boronic acid groups are attached to the benzyl ring and may be in the ortho- meta- or para-positions.

In one preferred embodiment, the quencher precursor (before incorporation into a hydrogel) may be 3,3′-oBBV (structure illustrated below); see U.S. Pat. No. 7,470,420, incorporated in its entirety herein by reference thereto.

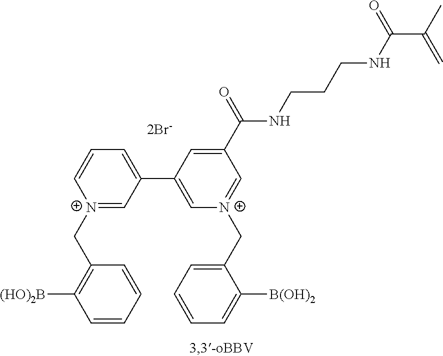

The quencher precursor 3,3′-oBBV may be used with HPTS-CysMA to make hydrogels in accordance with preferred aspects of the invention.

Other indicator chemistries, such as those disclosed in U.S. Pat. Nos. 5,176,882 to Gray et al. and 5,137,833 to Russell, can also be used in accordance with embodiments of the present invention; both of which are incorporated herein in their entireties by reference thereto. In some embodiments, an indicator system may comprise an analyte binding protein operably coupled to a fluorophore, such as the indicator systems and glucose binding proteins disclosed in U.S. Pat. Nos. 6,197,534, 6,227,627, 6,521,447, 6,855,556, 7,064,103, 7,316,909, 7,326,538, 7,345,160, and 7,496,392, 7,718,353, U.S. Patent Application Publication Nos. 2003/0232383, 2005/0059097, 2005/0282225, 2009/0104714, 2008/0311675, 2007/0136825, 2007/0207498, and 2009/0048430, and PCT International Publication Nos. WO 2009/021052, WO 2009/036070, WO 2009/021026, WO 2009/021039, WO 2003/060464, and WO 2008/072338 which are hereby incorporated by reference herein in their entireties.

For in vivo applications, the sensor is used in a moving stream of physiological fluid which contains one or more polyhydroxyl organic compounds or is implanted in tissue such as muscle which contains said compounds. Therefore, it is preferred that none of the sensing moieties escape from the sensor assembly. Thus, for use in vivo, the sensing components are preferably part of an organic polymer sensing assembly. Soluble dyes and quenchers can be confined by a selectively permeable membrane that allows passage of the analyte but blocks passage of the sensing moieties. This can be realized by using as sensing moieties soluble molecules that are substantially larger than the analyte molecules (molecular weight of at least twice that of the analyte or greater than 1000 preferably greater than 5000); and employing a selective semipermeable membrane such as a dialysis or an ultrafiltration membrane with a specific molecular weight cutoff between the two so that the sensing moieties are quantitatively retained.

Preferably the sensing moieties are immobilized in an insoluble polymer matrix, which is freely permeable to glucose. The polymer matrix is comprised of organic, inorganic or combinations of polymers thereof. The matrix may be composed of biocompatible materials. Alternatively, the matrix is coated with a second biocompatible polymer that is permeable to the analytes of interest.

The function of the polymer matrix is to hold together and immobilize the fluorophore and quencher moieties while at the same time allowing contact with the analyte, and binding of the analyte to the boronic acid. To achieve this effect, the matrix must be insoluble in the medium, and in close association with it by establishing a high surface area interface between matrix and analyte solution. For example, an ultra-thin film or microporous support matrix is used. Alternatively, the matrix is swellable in the analyte solution, e.g. a hydrogel matrix is used for aqueous systems. In some instances, the sensing polymers are bonded to a surface such as the surface of a light conduit, or impregnated in a microporous membrane. In all cases, the matrix must not interfere with transport of the analyte to the binding sites so that equilibrium can be established between the two phases. Techniques for preparing ultra-thin films, microporous polymers, microporous sol-gels, and hydrogels are established in the art. All useful matrices are defined as being analyte permeable.

Hydrogel polymers are used in some embodiments. The term, hydrogel, as used herein refers to a polymer that swells substantially, but does not dissolve in water. Such hydrogels may be linear, branched, or network polymers, or polyelectrolyte complexes, with the proviso that they contain no soluble or leachable fractions. Typically, hydrogel networks are prepared by a crosslinking step, which is performed on water-soluble polymers so that they swell but do not dissolve in aqueous media. Alternatively, the hydrogel polymers are prepared by copolymerizing a mixture of hydrophilic and crosslinking monomers to obtain a water swellable network polymer. Such polymers are formed either by addition or condensation polymerization, or by combination process. In these cases, the sensing moieties are incorporated into the polymer by copolymerization using monomeric derivatives in combination with network-forming monomers. Alternatively, reactive moieties are coupled to an already prepared matrix using a post polymerization reaction. Said sensing moieties are units in the polymer chain or pendant groups attached to the chain.

The hydrogels useful in this invention are also monolithic polymers, such as a single network to which both dye and quencher are covalently bonded, or multi-component hydrogels. Multi-component hydrogels include interpenetrating networks, polyelectrolyte complexes, and various other blends of two or more polymers to obtain a water swellable composite, which includes dispersions of a second polymer in a hydrogel matrix and alternating microlayer assemblies.

Monolithic hydrogels are typically formed by free radical copolymerization of a mixture of hydrophilic monomers, including but not limited to HEMA, PEGMA, methacrylic acid, hydroxyethyl acrylate, N-vinyl pyrrolidone, acrylamide, N,N′-dimethyl acrylamide, and the like; ionic monomers include methacryloylaminopropyl trimethylammonium chloride, diallyl dimethyl ammonium. chloride, vinyl benzyl trimethyl ammonium chloride, sodium sulfopropyl methacrylate, and the like; crosslinkers include ethylene dimethacrylate, PEGDMA, trimethylolpropane triacrylate, and the like. The ratios of monomers are chosen to optimize network properties including permeability, swelling index, and gel strength using principles well established in the art. In one embodiment, the dye moiety is derived from an ethylenically unsaturated derivative of a dye molecule, such as 8-acetoxypyrene-1,3,6-N,N′,N″-tris(methacrylamidopropylsulfonamide), the quencher moiety is derived from an ethylenically unsaturated viologen such as 4-N-(benzyl-3-boronic acid)-4′-N′-(benzyl-4-ethenyl)-dipyridinium dihalide (m-SBBV) and the matrix is made from HEMA and PEGDMA. The concentration of dye is chosen to optimize emission intensity. The ratio of quencher to dye is adjusted to provide sufficient quenching to produce the desired measurable signal.

In some embodiments, a monolithic hydrogel is formed by a condensation polymerization. For example, acetoxy pyrene trisulfonyl chloride is reacted with an excess of PEG diamine to obtain a tris-(amino PEG) adduct dissolved in the unreacted diamine. A solution of excess trimesoyl chloride and an acid acceptor is reacted with 4-N-(benzyl-3-boronic acid)-4′-N′-(2 hydroxyethyl)bipyridinium dihalide to obtain an acid chloride functional ester of the viologen. The two reactive mixtures are brought into contact with each other and allowed to react to form the hydrogel, e.g. by casting a thin film of one mixture and dipping it into the other.

In other embodiments, multi-component hydrogels wherein the dye is incorporated in one component and the quencher in another are preferred for making the sensor of this invention. Further, these systems are optionally molecularly imprinted to enhance interaction between components and to provide selectivity for glucose over other polyhydroxy analytes. Preferably, the multicomponent system is an interpenetrating polymer network (IPN) or a semi-interpenetrating polymer network (semi-IPN).

The IPN polymers are typically made by sequential polymerization. First, a network comprising the quencher is formed. The network is then swollen with a mixture of monomers including the dye monomer and a second polymerization is carried out to obtain the IPN hydrogel.

The semi-IPN hydrogel is formed by dissolving a soluble polymer containing dye moieties in a mixture of monomers including a quencher monomer and through complex formation with the fluorophore. In some embodiments, the sensing moieties are immobilized by an insoluble polymer matrix which is freely permeable to polyhydroxyl compounds. Additional details on hydrogel systems have been disclosed in U.S. Pat. No. 7,470,420, and US Patent Publications No. US2004/0028612 which are hereby incorporated by reference in their entireties.

The polymer matrix is comprised of organic, inorganic or combinations of polymers thereof. The matrix may be composed of biocompatible materials. Alternatively, the matrix is coated with a second biocompatible polymer that is permeable to the analytes of interest. The function of the polymer matrix is to hold together and immobilize the fluorescent dye and quencher moieties while at the same time allowing contact with the analytes (e.g., polyhydroxyl compounds, H⁺ and OH⁻), and binding of the polyhydroxyl compounds to the boronic acid. Therefore, the matrix is insoluble in the medium and in close association with it by establishing a high surface area interface between matrix and analyte solution. The matrix also does not interfere with transport of the analyte to the binding sites so that equilibrium can be established between the two phases. In one embodiment, an ultra-thin film or microporous support matrix may be used. In another embodiment, the matrix that is swellable in the analyte solution (e.g. a hydrogel matrix) can be used for aqueous systems. In some embodiments, the sensing polymers are bonded to a surface such as the surface of a light conduit, or impregnated in a microporous membrane. Techniques for preparing ultra-thin films, microporous polymers, microporous sol-gels, and hydrogels have been established in the prior art.

In one preferred embodiment, the boronic acid substituted viologen may be covalently bonded to a fluorescent dye. The adduct may be a polymerizable compound or a unit in a polymer. One such adduct for example may be prepared by first forming an unsymmetrical viologen from 4,4′-dipyridyl by attaching a benzyl-3-boronic acid group to one nitrogen and an aminoethyl group to the other nitrogen atom. The viologen is condensed sequentially first with 8-acetoxy-pyrene-1,3,6-trisulfonyl chloride in a 1:1 mole ratio followed by reaction with excess PEG diamine to obtain a prepolymer mixture. An acid acceptor is included in both steps to scavange the byproduct acid. The prepolymer mixture is crosslinked by reaction with a polyisocyanate to obtain a hydrogel. The product is treated with base to remove the acetoxy blocking group. Incomplete reaction products and unreacted starting materials are leached out of the hydrogel by exhaustive extraction with deionized water before further use. The product is responsive to glucose when used as the sensing component as described herein.

Alternatively, such adducts are ethylenically unsaturated monomer derivatives. For example, dimethyl bis-bromomethyl benzene boronate is reacted with excess 4,4′-dipyridyl to form a half viologen adduct. After removing the excess dipyridyl, the adduct is further reacted with an excess of bromoethylamine hydrochloride to form the bis-viologen adduct. This adduct is coupled to a pyranine dye by reaction with the 8-acetoxypyrene-tris sulfonyl chloride in a 1:1 mole ratio in the presence of an acid acceptor followed by reaction with excess aminopropylmethacrylamide. Finally, any residual amino groups may be reacted with methacrylol chloride. After purification, the dye/viologen monomer may be copolymerized with HEMA and PEGDMA to obtain a hydrogel.

### Solution Example

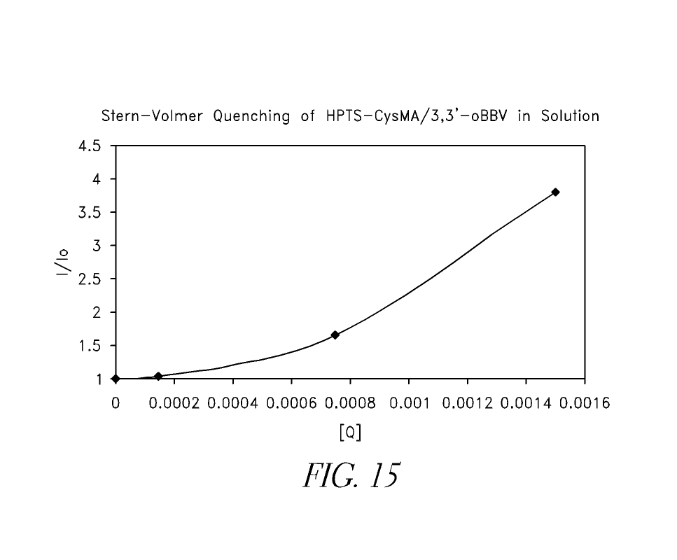

To a solution of HPTS-CysMA (1×10⁻⁵ M in pH 7.4 PBS) was added increasing amounts of 3,3′-oBBV (30 mM in MeOH) and the fluorescence emission measured after each addition. FIG. 15 gives the relative emission change (Stern-Volmer curve) upon addition of 3,3′-oBBV (Q) indicating the quenching of HPTS-CysMA with 3,3′-oBBV. The fluorimeter settings were as follows: 1% attenuation, ex slit 8 nm, em slit 12 nm, 486 nm ex λ, 537 nm em λ.

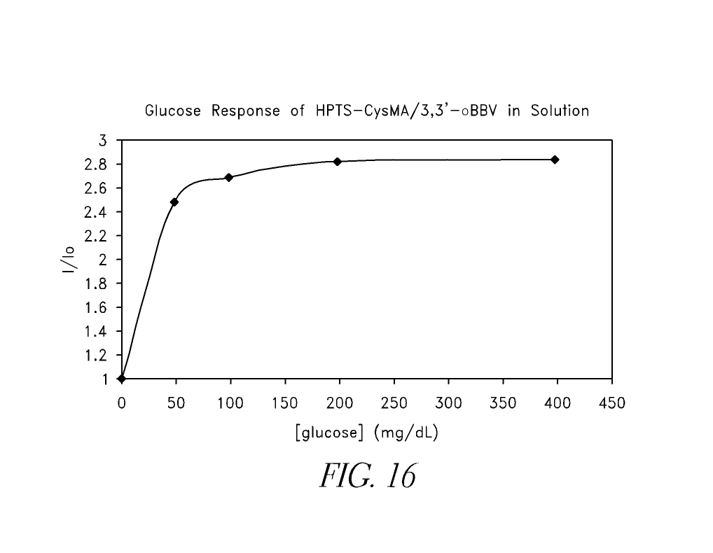

HPTS-CysMA (1×10-5 M) and 3,3′-oBBV (3×10-3 M) were titrated with a stock solution of glucose (31250 mg/dL) in pH 7.4 PBS and the fluorescence emission measured after each addition of glucose. The relative change upon addition of glucose is given in FIG. 16.

### Hydrogel Example

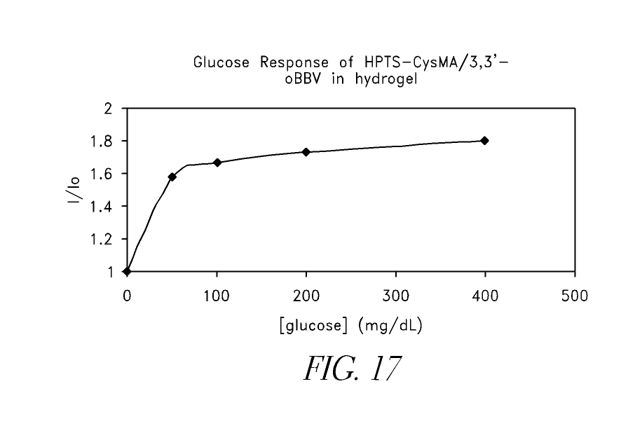

HPTS-CysMA (2 mg), 3,3′-oBBV (15 mg), N,N′-dimethylacrylamide (400 mg), N,N′-methylenebisacrylamide (8 mg), HCl (10 μL of 1 M solution), and VA-044 (1 mg) were dissolved in water and diluted to 1 mL in a volumetric flask. The solution was freeze-pump-thawed (3×), injected into a mold containing a 0.005″ polyimide spacer and polymerized at 55° C. for 16 h. The resultant film was placed in pH 7.4 phosphate buffer and was tested in a flow cell configuration with increasing amounts of glucose (0, 50, 100, 200, 400 mg/dL). The relative fluorescence change upon addition of glucose is given in FIG. 17. The fluorimeter settings were as follows: ex slit 8 nm, em slit 3.5 nm, 515 nm cutoff filter, 486 nm ex λ, 532 nm em λ.

### Thromboresistant Coatings

Molecules of a biocompatible agent are attached to the surfaces of the medical device to improve biocompatibility, such as antithrombogenic agents like heparin, albumin, streptokinase, tissue plasminogin activator (TPA) or urokinase. For example, the biocompatible agent may comprise molecules of both albumin and heparin. In one embodiment the molecules of a biocompatible material are joined to one another to form a film that is attached to a solid surface by a linking moiety. In other examples, various surface treatments of the optical glucose sensor can be used, such as those disclosed in U.S. Pat. Nos. 4,722,906, 4,973,493, 4,979,959, 5,002,582, 5,049,403, 5,213,898, 5,217,492, 5,258,041, 5,512,329, 5,563,056, 5,637,460, 5,714,360, 5,840,190, 5,858,653, 5,894,070, 5,942,555, 6,007,833, 6,090,995, 6,121,027, 6,254,634, 6,254,921, 6,278,018, 6,410,044, 6,444,318, 6,461,665, 6,465,178, 6,465,525, 6,506,895, 6,559,132, 6,669,994, 6,767,405, 7,300,756, 7,550,443, 7,550,444, and U.S. Patent Publ. Nos. 20010014448, 20030148360, and 20090042742 (each of which is incorporated herein in its entirety by reference thereto).

In one embodiment, the chemical linking moiety has the formula A-X-B in which A represents a photochemically reactive group capable of bonding covalently to a solid surface; B represents a different reactive group capable desirably in response to specific activation to which group A is unresponsive, of forming a covalent bond to a biocompatible agent and X represents a relatively inert, noninterfering skeletal moiety joining groups “A”, and “B”, that is resistant to cleavage in aqueous physiological fluid. The physiological fluid referred to is such fluid with which X will come in contact (e.g., blood, interstitial fluid, etc.). In a method of the invention group “A” of the linking moiety is covalently bound to the solid surface, with a sufficient population density to enable the molecules of the biocompatible agent to effectively shield the solid surface when the molecules are covalently bound to group “B” to provide a biocompatible effective surface. A biocompatible device of this invention includes a solid surface to which molecules of a biocompatible agent have been bound via the chemical-linking moiety as follows: solid surface-A residue-X-B residue-molecules of a biocompatible agent.

In one embodiment, the molecules of the biocompatible agent are selectively bound to the solid surface with a sufficient population density to provide a biocompatible effective surface using a chemically linking moiety that has the formula:

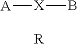

in which R represents a selector group that is a member of a specific bonding pair and that is reactive to form a bond with a receptor forming the other member of the specific binding pair and carried by a selected biocompatible agent and A, and B represent the groups described above as A and B. X represents a relatively inert, non-interfering skeletal radical joining groups “A”, “B” and “R” and sterically enabling group “B” to separate from group “R” by at least about 10 Å.

Groups “B” and “R” are preferably sterically distinct groups; that is, they may, during the course of thermal vibration and rotation separate by a distance of at least about 10 Å. Group R, a “selector” group, representing a member of a specific binding pair, commonly forms a bond, usually noncovalent, with the biocompatible agent at an epitopic or other binding site of the latter (which site typifies a “receptor” herein). The group “B”, which upon activation can covalently bond to the biocompatible agent, may be sterically spaced from the group “R”, thereby enabling the covalent bond to be formed at a site spaced from the receptor site. In turn, the selector receptor bond may be disassociated from the receptor site through breakage of a fragile bond between the selector group and the chemical linking moiety followed by removal of the selector by, e.g., dialysis, environmental changes (pH, ionic strength, temperature, solvent polarity, etc.) or through spontaneous catalytic modification of the selector group (as when the biocompatible agent is an enzyme), etc. The receptor thus is reactivated to permit subsequent reaction with members of the specific binding pair.

As referred to herein, “specific binding pair” refers to pairs of substances having a specific binding affinity for one another. Such substances include antigens and their antibodies, haptens and their antibodies, enzymes and their binding partners (including cofactors, inhibitors and chemical moieties whose reaction the enzymes promote), hormones and their receptors, specific carbohydrate groups and lectins, vitamins and their receptors, antibiotics and their antibodies and naturally occurring binding proteins, etc. The concept of employing specific binding pairs in analytical chemistry is well known and requires little further explanation. Reference is made to Adams, U.S. Pat. No. 4,039,652, Maggio, et al, U.S. Pat. No. 4,233,402 and Murray, et al, U.S. Pat. No. 4,307,071, the teachings of which are incorporated herein by reference.

In certain embodiments, X is preferably a C₁-C₁₀ alkyl group such as polymethylene, a carbohydrate such as polymethylol, a polyoxyethylene, such as polyethylene glycol or a polypeptide such as polylysine.

The reactive group B is preferably a group that upon suitable activation covalently bonds to proteinaceous or other biocompatible agents. Such groups are typified by thermochemical groups and photochemical groups, as described and exemplified in Guire, U.S. Pat. No. 3,959,078, the teachings of which are incorporated herein by reference.

The photochemically reactive groups (A) (the covalent bonding of which is activated by actinic radiation) may be typified by aryl, alkyl and acyl azides, oxazidines, isocyanates (nitrene generators), alkyl and 2 ketodiazo derivatives and diazirines (carbene generators), aromatic ketones (triplet oxygen generators), aromatic diazonium derivatives and numerous classes of carbonium ion and radical generators. Reference is made to Frederick J. Darfler and Andrew M. Tometsko, chapter 2 of Chemistry and Biochemistry of Amino Acids, Peptides and Proteins (Boris Weinstein, ed) vol. 5, Marcel Dekker, Inc. New York, 1978, for further description of photochemically reactive groups. Azidonitrophenyls, fluoroazido nitrobenzenes, and aromatic ketones form a preferred group due to their stability to chemical reaction conditions in the dark and their susceptibility to activation by light of wave lengths harmless to most biomaterials, to form short-lived reactive intermediates capable of forming covalent bonds in useful yield with most sites on the biomaterial.

Nitrophenylazide derivatives (shown as including the X group) appropriate for use as photochemically reactive groups for the most part can be derived from fluoro-2-nitro-4-azidobenzene, and include 4-azido-2-nitrophenyl(ANP)-4-aminobutyryl, ANP-6-aminocaproyl, ANP-11-aminoundecanoyl, ANP-glycyl, ANP-aminopropyl, ANP-mercaptoethylamino, ANP-diaminohexyl, ANP-diaminopropyl, and ANP-polyethylene glycol. ANP-6-aminocaproyl, ANP-11-aminoundecanoyl, and ANP-polyethylene glycol are preferred. Aromatic ketones preferred for use as photochemically reactive groups include benzylbenzoyl and nitrobenzylbenzoyl.

Thermochemical reactive groups (that are activated by heat energy) are typified by and include nitrophenylhalides, alkylamino, alkylcarboxyl, alkylthiol, alkylaldehyde, alkylmethylimidate, alkylisocyanate, alkylisothiocyanate and alkylhalide groups.

Groups appropriate for use as thermochemically reactive groups include carboxyl groups, hydroxyl groups, primary amino groups, thiol groups, maleimides and halide groups. N-oxysuccinimide carboxylic esters of such groups as 6-amino hexanoic acid and amino undecanoic acid, alkylthiol groups such as mercaptosuccinic anhydride and beta-mercaptopropionic acid, homocysteinethiolactones, and polyetheylene glycol derivatives are preferred.

Other linking agents can also be used in the embodiments of the present disclosure, such as those disclosed in U.S. Pat. No. 6,077,698, which is incorporated herein by reference. For example, a chemical linking agent comprising a di- or higher functional photoactivatable charged compound can be used. The linking agent preferably provides at least one group that is charged under the conditions of use in order to provide improved water solubility. The linking agent may further provide two or more photoactivatable groups in order to allow the agent to be used as a cross-linking agent in aqueous systems. In preferred embodiments, the charge is provided by the inclusion of one or more quaternary ammonium radicals, and the photoreactive groups are provided by two or more radicals of an aryl ketone such as benzophenone.

The thromboresistant agent may carry one or more latent reactive groups covalently bonded to them. The latent reactive groups are groups which respond to specific applied external stimuli to undergo active specie generation with resultant covalent bonding to an adjacent support surface. Latent reactive groups are those groups of atoms in a molecule which retain their covalent bonds unchanged under conditions of storage but which, upon activation, form covalent bonds with other molecules. The latent reactive groups generate active species such as free radicals, nitrenes, carbenes, and excited states of ketones upon absorption of external electromagnetic or kinetic (thermal) energy. Latent reactive groups may be chosen to be responsive to various portions of the electromagnetic spectrum, and latent reactive groups that are responsive to ultraviolet, visible or infrared portions of the spectrum are preferred. Latent reactive groups as described are generally well known.

The azides constitute a preferred class of latent reactive groups and include arylazides, such as those disclosed in U.S. Pat. No. 5,002,582, which is incorporated by reference herein, for example phenyl azide and particularly 4-fluoro-3-nitrophenyl azide, acyl azides such as benzoyl azide and p-methylbenzoyl azide, azido formates such as ethyl azidoformate, phenyl azidoformate, sulfonyl azides such as benzenesulfonyl azide, and phosphoryl azides such as diphenyl phosphoryl azide and diethyl phosphoryl azide. Diazo compounds constitute another class of latent reactive groups and include diazoalkanes (—CHN₂) such as diazomethane and diphenyldiazomethane, diazoketones such as diazoacetophenone and 1-trifluoromethyl-1-diazo-2-pentanone, such as t-butyl diazoacetate and phenyl diazoacetates, and beta-ketone-alpha-diazoacetates such as t butyl alpha diazoacetoacetate. Other latent reactive groups include the aliphatic azo compounds such as azobisisobutyronitrile, the diazirines such as 3-trifluoromethyl-3-phenyldiazirine, the ketenes (—CH═C═O) such as ketene and diphenylketene and photoactivatable ketones such as benzophenone and acetophenone. Peroxy compounds are contemplated as another class of latent reactive groups and include dialkyl peroxides such as di-t-butyl peroxide and dicyclohexyl peroxide and diacyl peroxides such as dibenzoyl peroxide and diacetyl peroxide and peroxyesters such as ethyl peroxybenzoate. Upon activation of the latent reactive groups to cause covalent bond formation to the surfaces to which polymer molecules are to be attached, the polymer molecules are covalently attached to the surfaces by means of residues of the latent reactive groups. Exemplary latent reactive groups are recited in U.S. Pat. No. 5,002,582 incorporated herein by reference.

The polymers and oligomers used may have one or more latent reactive groups. In certain embodiments, the polymers have at least one latent reactive group per molecule with the ratio of reactive groups extended polymer length, in Angstroms, ranging from about 1/10 to about 1/700 and preferably from about 1/50 to 1/400. As will be noted from the foregoing description, photoreactive latent reactive groups are for the most part aromatic and hence generally are hydrophobic rather than hydrophilic in nature.

The latent reactive groups and the polymer molecules to which they are bonded may have substantially different solvophilic properties. For example, the latent reactive groups may be relatively hydrophobic, whereas the polymer molecules may be relatively hydrophilic; when solution of the molecules is contacted with a relatively hydrophobic surface, it is believed that the latent reactive groups, being hydrophobic, tend to orient nearer the surface so as to improve bonding efficiency when the latent reactive groups are activated. The preferred latent reactive groups are benzophenones, acetophenones, and aryl azides.

The loading density of polymers upon a surface may be improved by a process in which a latent reactive molecule (a molecule having a latent reactive group) is first brought into close association (as by means of a solvent solution) to a surface, and thereafter the polymer to be bonded to the surface is brought into contact with and is covalently bonded to the latent reactive molecule, as to a reactive group different from the latent reactive group. Thereafter, the latent reactive groups may be activated to cause them to covalently bond to the surface to thereby link the polymers to the surface.

In other embodiments, polymer chains may be provided upon a surface or other substrate by first covalently bonding to the substrate through a latent reactive group a monomer, oligomer or other reactive chemical unit. To the thus bonded reactive units are covalently bonded monomers or oligomers in a polymerization reaction or polymers via covalent bonding (grafting) of the reactive units onto the polymer chains.

The reactive chemical units of the invention carry covalently bonded thereto latent reactive groups as described herein for covalent attachment to a non pretreated surface or other substrate. These molecules are characterized as having reactive groups capable of covalent bonding to polymer molecules of a polymer having the desired characteristics, or of entering into a polymerization reaction with added monomers or oligomers to produce polymer chains having the desired characteristics. Reactive chemical molecules capable of covalently bonding to polymer molecules include not only monomers and oligomers of various types but also molecules having such functional groups as carboxyl, hydroxyl, amino, and N-oxysuccinimide, such groups being reactive with reactive groups carried by the polymer chain to bond to the chain. The reactive chemical molecules are preferably monomers or oligomers and most preferably are ethylenically unsaturated monomers capable of entering into an addition polymerization reaction with other ethylenically unsaturated monomers. Particularly preferred are the acrylate and methacrylate monomers which are the esterification products of acrylic or methacrylic acid and hydroxy-functional latent reactive groups. Examples of such molecules include 4-benzoylbenzoyl-lysyl-acrylate.

Utilizing reactive chemical units bearing latent reactive groups, one may first coat a surface or other substrate with a solvent solution of such molecules. Upon removal of solvent, the application of an appropriate external stimulus such as U.V. light will cause the molecules to covalently bond, through the latent reactive groups, to the substrate. The substrate may then be appropriately contacted with a solution containing the desired polymer, monomer or oligomer molecules to cause bonding to these molecules. For example, if the reactive chemical unit molecule is carboxyl functional, it may be reactive with, and covalently bonded to, an appropriate hydroxyl-functional polymer such as dihydroxy polyethylene glycol. If the reactive chemical molecule is a monomer or oligomer, e.g., a methacrylate monomer, the substrate to which the molecule is covalently bonded may be contacted with a solution of addition-polymerizable monomers such as hydroxyethyl methacrylate and a free-radical addition polymerization initiator such as dibenzoyl peroxide under addition polymerization conditions to result in the growth of polymer chains from the monomer molecules bound covalently to the substrate. Once the desired polymerization has occurred, the substrate may be washed to remove residual monomer, solvent and non bound polymer that was formed.

In other embodiments the thromboresistant coating can adhere better by surface modification of the medical device by adsorbing a layer of a polyamine having a high average molecular weight on to the surface. The polyamine is stabilised by cross-linking with crotonaldehyde, which is a mono-aldehyde having a C—C double bond in conjugation with the aldehyde function. Thereafter one or more alternating layers of an anionic polysaccharide and the cross-linked polyamine, followed by a final layer of the said polyamine, not cross-linked, may be adsorbed onto the first layer of cross-linked polyamine, whereby a surface modification carrying free primary amino groups is achieved.

In certain embodiments, the thromboresistant coating is made by bringing the substrate into contact with an aqueous solution of the polyamine at pH 8-10, for example pH 9. The concentration of the initial polyamine solution will range from 1-10% by weight, especially 5% by weight, 1 ml of which may be diluted to a final volume of 500-2000 ml, especially 1000 ml. This final solution may also comprise from 100-1000 μl, especially 340 μl crotonaldehyde. Alternatively the substrate will be treated first with a solution of polyamine of said concentration and pH, and then with a solution of the crotonaldehyde of the said concentration and pH. The temperature is not critical, so it is preferred for the treatment to be at room temperature.

After rinsing with water, the substrate is treated with a solution of an anionic polysaccharide, containing from about 10 to about 500 mg, preferably about 100 mg of the polysaccharide in a volume of 1000 ml. This step is executed at a temperature in the range of 40°-70° C., preferably about 55° C. and pH 1-5, preferably about pH 3.

After another rinsing with water, these first steps may be repeated one or several times and finally, after having adsorbed a layer of polysaccharide, the substrate may be treated with a polyamine solution having a concentration 1-20 times, preferably 10 times, that mentioned above, at the said temperature and pH. The polyamine will preferably be a polymeric aliphatic amine, especially polyethylene imine having a high average molecular weight, but any polyamine having a high average molecular weight and carrying free primary amino groups may be used. The anionic polysaccharide will preferably be a sulfated polysaccharide. The aminated surface may optionally be further stabilized by reduction with a suitable reducing agent such as sodium cyanoborohydride. The modified surface according to present invention has free primary amino groups by which chemical entities may be bound either ionically or covalently. Also aldehyde containing chemical entities may be bound by formation of Schiff's bases, eventually followed by a stabilization reaction such as a reduction to convert the Schiff's bases to secondary amines. Further examples are disclosed in U.S. Pat. No. 5,049,403 which is hereby incorporated by reference in its entirety.

In certain embodiments, to provide a thromboresistant coating to the medical device, a composition is prepared to include a solvent, a combination of complementary polymers dissolved in the solvent, and the bioactive agent or agents dispersed in the polymer/solvent mixture. The solvent is preferably one in which the polymers form a true solution. The pharmaceutical agent itself may either be soluble in the solvent or form a dispersion throughout the solvent.

Due to the properties of materials frequently used on the outer surface of sensors, sensors can be difficult to coat with conventional anticoagulants, or anti-thrombogenics, e.g., heparin, to obtain a suitable anticoagulant coating, which is sufficiently stable, long-lasting, and active for preferred intravascular applications, and yet is sufficiently invisible to analytes of interest and non-interfering with the sensor chemistry to allow reliable and sufficiently long-lasting operation. Various issues can arise relating to the suitability of a particular coating including, for example, stability of the coating during manufacturing and handling of the sensor, resistance of the coating to removals during use, such as by solubilization, reaction, etc., resistance to diffusion through the coating of analytes of interest, and interaction between species in the coating and the sensor technology, whether by hydrolysis of detectable species from the coating or by other means.

Coating materials comprising heparin are preferred, but other polysaccharide and biologically derived materials and analogs can be utilized as well, either with heparin or in place of heparin. Preferred methods of applying the coating include application of a heparin-quaternary ammonium complex in isopropanol to a sensor wetted with water or water/surfactant under vacuum, but other suitable methods of applying a coating can also be successfully employed, such as application of a heparin-quaternary ammonium complex from combinations of solvents, such as non-polar solvents and polar solvents; sequential application of quaternary ammonium compound and heparin, such as to form a heparin-quaternary ammonium complex in-situ; covalently bonding heparin molecules to the surface of the sensor, including methods for attaching individual ends of heparin molecules to the surface such as described by Carmeda A B (Upplands Vasby, Sweden); and application of cross-linked forms of heparin or heparin with other compounds.

In certain embodiments, a coating of heparin or a heparin containing material can be applied to at least a portion of the sensor surface to limit or prevent thrombus formation. However, in some cases, application of such a coating can be difficult due to problems of adhesion where the coating will not properly adhere to the surface initially or will tend to detach or dissolve from the surface upon use. Instances where the coating detaches upon use can be particularly problematic due to the possibility of particulate impurities being released into the bloodstream and the possibility that these can result in plugging of small blood vessels. In addition, detachment or dissolution of heparin coating material can result in therapeutic or sub-therapeutic dosing of the patient with an anticoagulant material. Such adhesion problems can be particularly pronounced when applied to certain types of materials, especially plastics such as polyolefins, fluoropolymers, polycarbonate, and polysulfone. For example, polyolefins and fluoropolymers in particular are especially difficult to adhere materials to, as evidenced by the difficulty and limited strength that is typically achieved when these plastics are glued.

In one embodiment, a coating comprising heparin and benzalkonium can be effectively applied and can maintain an acceptably stable and active coating over polymeric surfaces of analyte sensors disclosed herein, including polymeric surfaces such as polyolefins, fluoropolymers, polycarbonate and polysulfone, porous polymeric surfaces, and porous polymeric surfaces on sensors incorporating immobilizing polymeric matrices, while still maintaining acceptable functionality of the analyte sensor. In certain embodiments, the porous surfaces capable of maintaining an acceptably stable and active coating comprising heparin and benzalkonium are more specifically described as microporous, nanoporous, or mesoporous.

In preferred embodiments, the coating comprising heparin and benzalkonium may include pharmaceutical grade heparin, such as heparin sodium or heparin calcium as described in the U.S. Pharmacopeia, revised Jun. 18, 2008, however, other grades and forms of heparin can be utilized in various applications, including instances where pharmaceutical regulations do not apply. Preferred grades of heparin can have an average molecular weight of about 12 to about 15 kDa, however, individual strands can have molecular weights as high as about 40 kDa or 50 kDA or even higher, and as low as about 5 kDa or 3 kDa or even lower. In other embodiments, heparin with average molecular weights higher or lower than about 12 to about 15 kDa can be successfully utilized, such as those as high as about 20 or 30 kDa or as low as about 7 or 10 kDa.

In some preferred embodiments, the coating comprising heparin and benzalkonium may include molecules of benzalkonium chloride having alkyl groups of about 1 to about 5 carbons for two of the R-groups and an alkyl group of about six to about 22 carbons for the third R-group, either as a single pure compound or as a combination of compounds with differing R-groups. In some embodiments, grades of benzalkonium chloride include those having compounds and mixtures of compounds having primarily two methyl groups and an alkyl group of about six to about 22 carbons, or more preferably two methyl groups and an alkyl group of about 10 to about 18 carbons as the R-groups.

In certain embodiments, other ammonium complexes can be used, e.g., particular alkylbenzyl dimethyl ammonium cationic salts, which can be used in high loading concentrations with heparin to form coatings, as disclosed in U.S. Pat. No. 5,047,020 to Hsu; incorporated herein in its entirety by reference. Hsu found that commercially available benzalkonium chloride may comprise a mixture of alkylbenzyldimethylammonium chloride of the general formula, [C₆H₅CH₂N(CH₃)₂R]Cl, in which R represents a mixture of alkyls, including all or some of the groups comprising C8 and greater, with C12, C14 and C16 comprising the major portion. Generally, the composition breaks down to more than 20% C14, more than 40%, C12 and a less than 30% mixture of C8, C10 and C16. In contrast, Hsu found that preferred heparin/quaternary ammonium complexes have at least about 50 weight percent of the organic cationic salt, and preferably from 60 to 70 weight percent. Hsu found that optimum results were achieved with complexes consisting of cetalkonium heparin and/or stearylkonium heparin and mixtures thereof. Indeed, Hsu teaches that coatings for medical devices consisting of complexes of cetalkonium heparin and/or stearylkonium heparin and mixtures thereof, exhibit “vastly superior hydrophobicity and surface adhesion over the presently and most commonly used complexes of heparin and benzalkonium chloride.” Accordingly, in another aspect of the invention, other heparin/quaternary ammonium complexes besides those comprising benzalkonium, like those disclosed by Hsu, may be used to coat and render thromboresistant the glucose sensors disclosed herein.

### Surface Coating Agents

Various compounds can be useful as coating agents for the thromboresistant coating of the medical device, for example those disclosed in U.S. Pat. Nos. 6,278,018, 6,603,040, 6,924,390, 7,138,541, which are all incorporated herein by reference. In one aspect, the present invention provides a compound comprising a nonpolymeric core molecule comprising an aromatic group, the core molecule having attached thereto, either directly or indirectly, one or more substituents comprising negatively charged groups, and two or more photoreactive species, wherein the photoreactive species are provided as independent photoreactive groups. The first and second photoreactive species of the present coating agent can, independently, be identical or different.

In certain embodiments the core is provided as the residue of a polyhydroxy benzene starting material (e.g., formed as a derivative of hydroquinone, catechol, or resorcinol), in which the hydroxy groups have been reacted to form an ether (or ether carbonyl) linkage to a corresponding plurality of photogroups. In one embodiment, a coating agent of this invention further comprises one or more optional spacers that serve to attach a core molecule to corresponding photoreactive species, the spacer being selected from radicals with the general formula: wherein n is a number greater or equal to 1 and less than about 5, and m is a number greater or equal to 1 and less than about 4.

In another embodiment, such coating agents are selected from the group 4,5-bis(4-benzoylphenylmethyleneoxy)benzene-1,3-disulfonic acid di(potassium and/or sodium) salt, 2,5-bis(4-beizoylphenylmethyleneoxy)benzene-1,4-disulfonic acid di(potassium and/or sodium) salt, 2,5-bis(4-benzoylphenylmethyleneoxy)benzene-1-sulfonic acid monopotassium and/or monosodium salt.

Suitable core molecules of the present invention include nonpolymeric radicals having a low molecular weight (e.g., 100-1000 MW). Suitable core molecules provide an improved combination of such properties as coating density, structural stability, ease of manufacture, and cost. Further, core molecules can be provided with water soluble regions, biodegradable regions, hydrophobic regions, as well as polymerizable regions. Examples of suitable core molecules include cyclic hydrocarbons, such as benzene and derivatives thereof.

The type and number of charged groups in a preferred coating agent are sufficient to provide the agent with a water solubility (at room temperature and optimal pH) of at least about 0.1 mg/ml, and preferably at least about 0.5 mg/ml, and more preferably at least about 1 mg/ml. Given the nature of the surface coating process, coating agent solubility levels of at least about 0.1 mg/ml are generally adequate for providing useful coatings of target molecules (e.g., polymer layers) on surfaces.

The coating agent can thus be contrasted with many coating agents in the art, which are typically considered to be insoluble in water (e.g., having a comparable water solubility in the range of about 0.1 mg/ml or less, and more often about 0.01 mg/ml or less). For this reason, conventional coating agents are typically provided and used in solvent systems in which water is either absent or is provided as a minor (e.g., less than about 50% by volume) component.

Examples of suitable charged groups include salts of organic acids (e.g., sulfonate, phosphonate, and carboxylate groups), as well as combinations thereof. A preferred charged group for use in preparing coating agents of the present invention is a sulfonic acid salt, e.g., derivatives of SO₃⁻ in which the counterion is provided by the salts of Group I alkaline metals (Na, K, Li ions) to provide a suitable positively charged species.

The use of photoreactive species in the form of photoreactive aryl ketones are preferred, such as acetophenone, benzophenone, anthraquinone, anthrone, and anthrone-like heterocycles (i.e., heterocyclic analogs of anthrone such as those having N, O, or S in the 10-position), or their substituted (e.g., ring substituted) derivatives. Examples of preferred aryl ketones include heterocyclic derivatives of anthrone, including acridone, xanthone, and thioxanthone, and their ring substituted derivatives. Particularly preferred are thioxanthone, and its derivatives, having excitation energies greater than about 360 nm.

The functional groups of such ketones are preferred since they are readily capable of undergoing the activation/inactivation/reactivation cycle described herein. Benzophenone is a particularly preferred photoreactive moiety, since it is capable of photochemical excitation with the initial formation of an excited singlet state that undergoes intersystem crossing to the triplet state. The excited triplet state can insert into carbon-hydrogen bonds by abstraction of a hydrogen atom (from a support surface, for example), thus creating a radical pair. Subsequent collapse of the radical pair leads to formation of a new carbon-carbon bond. If a reactive bond (e.g., carbon-hydrogen) is not available for bonding, the ultraviolet light-induced exitation of the benzophenome group is reversible and the molecule returns to ground state energy level upon removal of the energy source. Photoactivatible aryl ketones such as benzophenone and acetophenone are of particular importance inasmuch as these groups are subject to multiple reactivation in water and hence provide increased coating efficiency.

### Coating Methodology

The coating processes disclosed herein include: 1) direct coating of the heparin complex by straight application, as in the case of dip coating, as well as 2) indirect coating, as in the case of sequential applications of a quarternary ammonium salt plus surfactant and the ionic heparin. Suitable methods for applying a coating comprising heparin and benzalkonium may include multistep layering techniques as well as single step application of heparin complexes. In other embodiments, pretreatment methods are used, such as soaking the sensors in sodium heparin solutions.

In the event that it is desired to apply the thromboresistant coating to surfaces that are inert to certain polymeric materials, adhesion can be facilitated by chemically treating the surfaces in order to provide hydroxyl groups on or near the surface thereof. Exemplary chemical surface treatments in this regard include such known procedures as chemical etching, surfactant adsorption, coextrusion, plasma discharge, surface oxidation or reduction, radiation activation and oxidation, and surface grafting with materials such as polyvinyl alcohol, poly(2-hydroxyethyl methacrylate) and the like. Bulk modifications of the substrate surface can also be accomplished in order to provide active hydrogens. Examples of conventional modifications of this type include blending with polymers having active hydrogens, partial degradation of polymers, end group modification, monomer functionalization, oxidation, reduction, copolymerization, and the like.

In certain embodiments, a three-dimensional highly crosslinked matrix containing aminosilanes is formed on the medical device surface. The aminosilane is cured, crosslinked or polymerized in place on the surface to be rendered thromboresistant. This is carried out in a manner such that a three-dimensional matrix is formed. The matrix can be either an aminosilane homopolymer or a copolymer, including a graft copolymer, of an aminosilane with another silane that is not an aminosilane. Representative aminosilanes include 3-aminopropyltrimethoxysilane, 3-aminopropyltriethoxysilane, 2-aminoundecyltrimethoxysilane, aminophenyltrimethoxysilane, N-(2-aminoethyl-3-aminopropyl)trimethoxysilane, and trimethoxysilylpropyldiethylenetriamine.

Aminosilanes of this type can be used alone in order to form a homopolymer matrix. For example, certain aminosilanes are trifunctional and provide a highly crosslinked matrix. The hydrophilicity can be reduced, when desired, by combining the hydrophilic aminosilane with a less hydrophilic silane that is not an aminosilane. In one embodiment, a matrix that is a copolymer of one of these aminosilanes with another silane molecule that is not an aminosilane and that is less hydrophilic than an aminosilane in order to thereby adjust the hydrophilicity of the matrix. Other methods and coating agents are also known in the art, including U.S. Pat. Nos. 5,053,048, 4,973,493, 5,049,403, all of which are incorporated by reference herein.

In preferred embodiments, a coating comprising heparin and benzalkonium is applied by first wetting the sensor surface with water or a combination of water and surfactant. Preferred surfactants include anionic surfactants, however other surfactants such as cationic surfactants or non-ionic surfactants may also be successfully employed in some embodiments. In particular, suitable surfactants include sodium laurel sulfate, sodium dodecyl sulfate, ammonium lauryl sulfate, sodium laureth sulfate. The wetted sensor is then treated with an alcoholic solution of heparin-quaternary ammonium complex. In certain embodiments, the alcoholic solution comprises isopropanol, however other alcohol based solutions may be used as well, depending on the embodiment. Preferred solutions of isopropanol may include about 1 to about 99% (wt.) of heparin-benzalkonium complex in isopropanol, including 5%, 10%, 25%, 50%, 75%, 90%, and 95% (and also including ranges of weight percentages bordered on each end by these recited weight percentages). One preferred solution of heparin-benzalkonium in isopropanol is manufactured by Celsus Laboratories, Inc. 12150 Best Place, Cincinnati Ohio 45241, under product number BY-3189 (described as Benzalkonium heparin solution in isopropyl alcohol, 887 U/mL). The wetted sensor can be dipped in the heparin-benzalkonium solution, or it can be sprayed onto the surface of the sensor or applied by another suitable technique. The sensor with coating solution applied is then dried. Additional coating material, such as to improve consistency of a coating or to thicken a coating, can be applied by dipping, spraying or other suitable means. When material is applied, preferred methods include those where the sensor is exposed to the heparin-benzalkonium solution for only a limited time, such as less than one minute, or less than about 30 seconds or about 10 seconds or even about 1 or 2 seconds, such as by dipping the sensor into the solution for only about a second (and also including time intervals bordered on the high end and the low end by the recited durations such as dipping the sensor into the solution for between 10 and 30 seconds). In some embodiments, short time intervals can prevent undesirable results, such as excessive solubilization of material from the sensor surface or excessive dehydration of the sensor. However, in some embodiments, longer time periods can successfully be utilized by, for example, increasing the concentration of heparin-benzalkonium concentration of the solution or by supplementing the solution with additional benzalkonium material or heparin material, or by adjusting the pH, or ionic strength of the solution. In some embodiments, during the coating process, the sensor can be rehydrated as needed or desired by application of water or a combination of water and surfactant and/or solvent.

However, other methods of applying a coating comprising heparin and benzalkonium can also be successfully employed. Suitable multistep layering techniques include those techniques where an heparin and benzalkonium are applied by a process comprising application of a suitable form and grade of benzalkonium chloride followed by application of a suitable form and grade of heparin. Any suitable solvent or combinations of solvents can be used for heparin, such as water or aqueous alcohol, and for benzalkonium chloride, such as nonpolar organic solvents (for example, toluene, petroleum ether, etc.). Preferred heparin solutions include those comprising heparin in a weight percentage of about 0.05%, 1%, 5%, 10%, 25%, 50%, 75%, 90%, and 95% (and also including ranges of weight percentages bordered on each end by these recited weight percentages). In certain such embodiments, a preferred heparin solution comprises a weight percent of heparin between about 0.05% to about 1%. Preferred benzalkonium chloride solutions include those comprising benzalkonium in a weight percentage of about 0.05%, 1%, 5%, 10%, 20%, 25%, 50%, 75%, 90%, and 95% (and also including ranges of weight percentages bordered on each end by these recited weight percentages). In certain such embodiments, a preferred benzalkonium chloride solution comprises a weight percent of benzalkonium chloride between about 1.0% to about 20%.

Other suitable coating techniques are described, for example, in U.S. Pat. Nos. 3,846,353, to Grotta, and 5,047,020, to Hsu, incorporated by reference herein in their entireties.

Single step application of heparin complexes can comprise applying a solution comprising heparin and benzalkonium of a suitable grade and form to the sensor, such as is described in U.S. Pat. No. 5,047,020, to Hsu. In certain embodiments, the solution may include benzalkonium chloride. Suitable solvents for the heparin and benzalkonium include those comprising polar organic solvents, alone or as mixtures, such as alcohols (e.g. isopropanol), halogenated solvents (e.g. trifluoro-trichloro ethane), etc. In some embodiments, the solution to be applied to the polymeric surface may include heparin and benzalkonium in a combined weight percentage of 0.1%, 1%, 5%, 10%, 25%, 50%, 75%, or about 90% of the total weight of the solution (also including ranges of weight percentages bordered on each end by these recited weight percentages). In certain such embodiments, a solution may contain between about 0.1% to about 75% heparin/benzalkonium by weight. In some embodiments, successive layers of heparin/benzalkonium complex can be applied to the surface of the sensor, for example, to build up a coating having a desired thickness and/or durability.

In certain embodiments, the distal portion of a pre-wetted sensor is dipped in a solution comprising heparin and benzalkonium in isopropanol, preferably for about 0.1 to about 30 seconds, and more preferably for about 1 to about 10 seconds, and even more preferably for about 1 second. In certain embodiments, the dipped sensor is subsequently air dried, preferably for at least about 10 seconds, and more preferably for about 0.5 minutes to about 10 minutes, and even more preferably for about 1 minute. The heparin/benzalkonium coating and drying steps are repeated in accordance with various embodiments, preferably from about 1 to about 20 times, or more preferably from about 2 to about 10 times, or even more preferably from about 3 to about 8 times, and even more preferably still from about 4 to about 6 times.

In certain embodiments, a sustained release of heparin from the sensor surface into the surrounding vessel is achieved by soaking the sensor. In one embodiment, the sensor, which optionally contains a hydrogel underneath the optional microporous membrane, is soaked in a solution of heparin for infusion of heparin into the swollen hydrogel. In one embodiment, an aqueous solution of at least about 10% sodium heparin is used. In a more preferred embodiment, an aqueous solution of at least about 20% sodium heparin is used. In a most preferred embodiment, an aqueous solution of at least about 30% sodium heparin is used. In other embodiments, other organic solvents and other forms of heparin may be used. In one embodiment, the sodium heparin solution is in phosphate buffered saline of about pH 5. After soaking for enough time to saturate the hydrogel, the sensor is removed from the solution and allowed to dry. In one embodiment, the sensor is soaked for at least about 1 hour. In a preferred embodiment, the sensor is soaked for about 2 hours. In one embodiment, the sensor is soaked for at least about 3 hour. When the sensor is then deployed in-vivo, the hydrogel re-swells in the bloodstream thus releasing the heparin gradually over time.

Additional steps can be utilized as necessary, such as, for example, cleaning the surface of the sensor with suitable agents such as solvents, surfactants, etc. and/or drying the coating, such as with a gas stream, or with heat, or with a heated gas stream, or with one or more dehydrating agents. In some embodiments, it is desirable to package the sensor as soon as possible after coating, since in some embodiments, after coating, the surface of the sensor may be somewhat tacky, and it may tend to pick up particulate matter.

Other methods of applying a heparin-based coating to the sensor includes covalently bonding heparin, or a heparin derivative, to the surface of the sensor or to an intermediate material applied to the surface of the sensor. Suitable techniques include those that covalently bond the end of a heparin molecule to the surface of the sensor or an intermediate, such as the techniques utilized by Carmeda A B (Upplands Vasby, Sweden). Other suitable methods also include those utilizing photoimmobilization to attach heparin, or a heparin derivative to the surface of a sensor or an intermediate material applied to the surface of the sensor, such as are described herein and by Surmodics (Eden Prairie, Minn.), as well as those depositing heparin complexes with polar and nonpolar solvents, such as are described in U.S. Pat. No. 6,833,253 to Roorda, et al.

### Calibration

The controller or monitor may further comprise certain calibration mechanisms, such as those described in U.S. Patent Publication No. 2010/0312483. For example, the sensors may be characterized by a modified version of the Michaelis-Menten equation from enzyme kinetics. The equation can be applied to various chemical and biological sensing systems, such as covalently or electrostatically reacting fluorophore sensors, enzymetic fluorophore sensors, or other polymer or macromolecule systems. Systems which follow the reaction mechanism described by Scheme I conform to what is commonly referred to as Michaelis-Menten kinetics and may be described by what is commonly referred to as the Michaelis-Menten equation. See Conners, K. A. Binding Constants: The Measurement of Molecular Complex Stability, John Wiley & Sons, Inc., New York, 1987.

Scheme I

Certain embodiments of the analyte sensors disclosed herein, and in particular the glucose sensors disclosed herein, require calibration before they can be properly used to generate meaningful readings of analyte concentration. A calibration equation is useful to establish the mathematical relationship between the measured fluorescent intensity and the analyte concentration being estimated. For example, for the case of a glucose sensor, once a calibration equation has been determined, it may be used to measure a glucose concentration from a measured fluorescent intensity.

In particular, for the case of a glucose sensor chemistry, the fluorescent response (I) of the chemistry to glucose (G) can be described by a modified form of the Michaelis-Menten equation in which three parameters (a, b, and c) are determined:

I=a+b*G/(c+G)

where “a” is the fluorescent signal intensity in the absence of glucose, “b” determines the asymptotic intensity at infinite glucose (minus the signal at zero concentration, “a”), and “c” gives the glucose concentration at which the intensity is one-half the difference between the asymptotic value and the background, i.e. a+b/2. The “c” parameter is thus analogous to the Michaelis-Menten constant, Km, in enzyme kinetic systems.

It should be noted that for certain embodiments of the glucose sensing chemistry, as with some other chemical systems conforming to Michaelis-Menten kinetics, the value of the Michaelis-Menten constant, Kₘ, or the “c” parameter is a substantially fixed property of the chemistry. In one embodiment, the “c” parameter is determined by the binding constant between glucose and the Receptor-Quencher.

In certain such methods, the Michaelis-Menten parameters are determined in reference to an analyte sensors based on lifetime chemistry. As in many chemical systems, the response of the sensor chemistry can be sensitive to other chemical parameters in addition to the analyte of interest.

In one embodiment, the Michaelis-Menten parameters are determined from a set of in vitro measurements of the fluorescent intensity using one or more solutions of known glucose concentrations. In certain such methods, it is advantageous that the solutions be held at or near physiological pH and temperature (7.4 and 37 degree centigrade, respectively), or calibrate the sensor with reference to the pH and temperature measurements. In one embodiment, the sensor is inserted into a test chamber and exposed to one glucose concentrations. The resulting data can be used to analytically determine the corresponding Michaelis-Menten parameters. Alternatively or in addition, the corresponding Michaelis-Menten parameters may be determined numerically from the resulting data using the method of least squares. This equation thus converts a measured fluorescent intensity, I, into a glucose value, G. It can be used in both in vitro and in vivo applications under the assumption that the fluorescent intensity does not change substantially (for a given glucose concentration) between the determination of the Michaelis-Menten parameters and the experimental application.

In one embodiment, an additional calibration step is used to compensate for changes in the fluorescent intensity that occur between the determination of the Michaelis-Menten parameters and the use of the sensor in an in vitro or in vivo application. This calibration could, in principle, be done at the bedside of a patient immediately before insertion of the sensor into a vein or artery. A temperature control feedback loop can be used to maintain the calibration solutions at an equivalent stable physiological temperature for the duration of the calibration procedure.

In another embodiment, the initial values of the Michaelis-Menten equation parameters are determined by the manufacturer or vendor, for example in a laboratory at the factory during manufacturing or prior to packaging, instead of by the end user or consumer, for example by a clinician at the patient's bedside. In this case, the Michaelis-Menten parameters can be determined from a multipoint in situ calibration in which, again, both the pH and temperature are carefully controlled. The initial determination of the Michaelis-Menten parameter values can thus be regarded as a factory calibration.

In some embodiments, Michaelis-Menten parameters determined for one manufactured sensor can be successfully used with another manufactured sensor to provide sufficiently accurate results. Accordingly, it may be efficient, cost effective, and sufficiently accurate to employ one set of Michaelis-Menten parameters for both sensors. Alternatively, a set of Michaelis-Menten parameters for an entire manufactured batch of sensors may be determined by calibrating a select few of the sensors, and averaging the resulting values to obtain a set of Michaelis-Menten parameters for the batch.

The monitor can utilize one or more values of the calibration parameters, including the Michaelis-Menten parameters (a, b and c), and the correction factors c_{A} and c_{F}, as described in U.S. Patent Publication Nos. 2010/0312483, which is hereby incorporated by reference. In some embodiments, values of calibration parameters can be preloaded into the monitor, such as by storing one or more value in memory, whether by the user or another party. In some embodiments, one or more values of the calibration parameters can be entered after following a calibration procedure, either with the analyte sensor functionally connected to the monitor or with the analyte sensor connected to a different monitor or reading device. In some embodiments, when a different monitor or reading device is used for calibration, information relating to the calibration can be communicated directly or indirectly between the monitor and the different monitor or reading device functionally connected to the analyte sensor during calibration. In some embodiments, the monitor will receive information relating to the measurement of analyte concentrations as determined with a different device or by a different technique, and use the information during calibration. In some instances, the information can be sent to the monitor with manual entry, such as by keyboard or touchscreen or other manual methods; or by direct or indirect communication with a separate device determining the analyte concentration; or by reading values from an information storage medium such as scanning written or printed information, scanning barcodes, reading magnetic, optical, or computer storage medium including disks, strips, RAM, flash drives, etc.

In some embodiments, the monitoring system can be integrated into a network including other devices such as additional monitors, displays including remote displays, televisions, data entry locations, computers, PDAs, telephones, monitoring stations, doctor offices, hospitals, etc. Networking can be via the Internet, local area network, wide-area network, secure network, private network, wireless networks, etc.

Embodiments of the analyte measuring devices disclosed herein may configure light sources, detectors, and one or more sensors in a variety of ways, particularly when the analyte measuring device includes an indicator system utilizing multiple fluorophores. Various analyte sensors disclosed herein are configured to provide improved estimates of the analyte concentration of a particular solution by taking the temperature, pH, or other parameter of the particular solution into account. Accordingly, some embodiments of the analyte sensors disclosed herein may include a temperature sensing element configured to generate a signal indicative of a temperature of the sample.

### Methods of Estimating Analyte Concentration Incorporating Temperature Correction

Some embodiments of the devices disclosed herein generate a signal indicative of analyte concentration which exhibits a temperature dependence. For example, if two solutions of precisely the same analyte concentration are measured at two different temperatures with the same measurement device, in some embodiments, the measurement device may generate differing signals indicative of the two analyte concentrations. Thus, the accuracy of determining a solution's true analyte concentration based on such as signal may be improved by taking the temperature of the solution into account.

It has been discovered that for some embodiments of the measurement devices disclosed herein, and in particular, for glucose measurement devices employing a quencher binding moiety operably coupled to a fluorophore, the temperature dependence of the fluorescent signal approximately follows a modified version of the classic Michaelis-Menten equation from enzyme kinetics:

$$[ Glu ] = \frac{{c}_{T} * [ {G}_{i} - {a}_{T} ]}{{a}_{T} + {b}_{T} - {G}_{i}} \qquad ( Equation 1 )$$

where
[Glu] is the calculated glucose concentration, at a temperature T,
a_{T} is the first Michaelis-Menten parameter “a”, at a temperature T,
b_{T} is the second Michaelis-Menten parameter “b”, at the same temperature T,
c_{T} is the third Michaelis-Menten parameter “c”, at the same temperature T, and
Gᵢ is the fluorescent signal (i=1,2), either referenced or unreferenced, where G₁ is the fluorescence emission at 583 nm when the fluorophore is excited at 470 nm (which is the absorption maximum of the fluorophore's base-form), and G₂ is the fluorescence emission at 583 nm when the fluorophore is excited at 420 nm or 430 nm (which is the absorption maximum of the fluorophore's acid-form). Note, however, that other combinations of excitation and emission wavelengths are also feasible for use in Equation 1.

Various embodiments of the measurement devices disclosed herein employ a quencher-fluorophore indicator system which measures analyte concentration through the establishment of an equilibrium between the analyte of interest, the binding moiety (e.g. quencher), and the fluorophore. In such a system, analyte concentration is not measured by enzymatic consumption or conversion of the analyte. In contrast, the classic Michaelis-Menten equation specifically describes enzyme kinetics, a non-equilibrium phenomena involving the consumption/conversion of the enzyme's substrate by the enzyme. Therefore, it is not to be expected, indeed it is surprising, that an equation closely related to the classic Michaelis-Menten equation would effectively describe the temperature dependence of these types of quencher-fluorophore-based measurement devices and analyte sensing elements (or other measurement devices and analyte sensing elements functioning through analogous equilibrium mechanisms). In any event, knowledge that these devices (and similar devices) exhibit a temperature dependence which follows a modified Michaelis-Menten equation allows the use of temperature correction methods and algorithms to improve the accuracy of analyte concentration measurements. Such methods and algorithms are disclosed herein, along with measurement devices which implement such methods and algorithms.

Accordingly, some embodiment methods of estimating an analyte concentration include generating a signal indicative of analyte concentration and a signal indicative of temperature. Since, in some embodiments, the signal indicative of analyte concentration exhibits the temperature dependence just described, in some embodiments, the signal indicative of temperature may be used to adjust the signal indicative of analyte concentration to correct for temperature dependence. Thus, in certain such embodiments, the methods further include transforming the signal indicative of the analyte concentration utilizing an equation of the form of a modified Michaelis-Menten equation, such as Equation 1 above, depending on Michaelis-Menten parameters, such as the parameters “a”, “b”, and “c”, as described above with reference to Equation 1.

The temperature dependence of Equation 1 is exhibited through the Michaelis-Menten parameters a_{T}, b_{T}, and c_{T}, as indicated by the subscript “T” labeling these parameters. In some embodiments, the temperature dependence may need to be determined through a temperature calibration. Thus, in certain embodiment methods, the values of one or more of the Michaelis-Menten parameters may be set based on data which includes temperature calibration data and the signal indicative of a temperature.

For example, in some embodiment methods, the temperature calibration data may be generated by a temperature calibration method. The temperature calibration method may include selecting a first test analyte sensing element, and creating and/or providing a set of at least three solutions of differing known analyte concentrations. In certain such embodiments, a first temperature is selected (T1), three solutions of the set of at least three solutions are heated and/or cooled to a temperature substantially similar to the selected first temperature, and a first set of at least three signals is generated using the first test analyte sensing element, each signal indicative of the concentration of analyte in a different one of the three solutions at the first temperature. Measurements are then made at a second temperature. Thus, in certain embodiments, a second temperature is selected (T2), three solutions of the set of at least three solutions (each of the three may be the same or different than a solution chosen for the first temperature) are heated and/or cooled to a temperature substantially similar to the selected second temperature, and a second set of at least three signals is generated using the first test analyte sensing element, each signal indicative of the concentration of analyte in a different one of the three solutions at the second temperature. Of course, more than three solutions may be used in either of these steps. And more than two temperatures may also be employed. Generally, the more solutions of differing concentration and the greater number of different temperatures that are employed, the greater the accuracy of the resulting calibration data.

Once the solutions having known analyte concentrations have been measured, and the first and second sets of at least three signals have been generated, in some embodiments, the sets of signals are used to determine (usually approximately) the relationship between one or more of the Michaelis-Menten parameters and temperature. For example, in some embodiments, the temperature calibration method may further include computing values of each of a first, second, and third Michaelis-Menten parameter at the first temperature (a_{T1}, b_{T1}, and c_{T1}) by an algorithm comprising fitting a modified Michaelis-Menten equation to a first fit dataset comprising the first set of at least three signals. In certain such embodiments, the temperature calibration method may further include computing values of each of a first, second, and third Michaelis-Menten parameter at the second temperature (a_{T2}, b_{T2}, and c_{T2}) by an algorithm comprising fitting a modified Michaelis-Menten equation to a second fit dataset comprising the second set of at least three signals. Thus, in methods such as these, each of the three Michaelis-Menten parameters has been determined at least two temperatures, providing data which may be used to create a model of the temperature dependence of each of the three Michaelis-Menten parameters.

To model the temperature dependence of the Michaelis-Menten parameters, in some embodiments, the temperature calibration method may further include selecting an equation relating the first Michaelis-Menten parameter (a_{T}) to temperature, the equation depending on a first set of temperature calibration parameters; and setting a value for each calibration parameter of the first set of calibration parameters based on the value of the first Michaelis-Menten parameter at the first temperature (a_{T1}) and the value of the first Michaelis-Menten parameter at the second temperature (a_{T2}). In some embodiments, similar steps are performed with respect to the second and third Michaelis-Menten parameters (b_{T} and c_{T}). Thus, for example, the temperature calibration method may further include selecting an equation relating the second Michaelis-Menten parameter (b_{T}) to temperature, the equation depending on a second set of temperature calibration parameters; and setting a value for each calibration parameter of the second set of calibration parameters based on the value of the second Michaelis-Menten parameter at the first temperature (b_{T1}) and the value of the second Michaelis-Menten parameter at the second temperature (b_{T2}). Similarly, in some embodiments, the temperature calibration method may further include selecting an equation relating the third Michaelis-Menten parameter (c_{T}) to temperature, the equation depending on a third set of temperature calibration parameters; and setting a value for each calibration parameter of the third set of calibration parameters based on the value of the third Michaelis-Menten parameter at the first temperature (c_{T1}) and the value of the third Michaelis-Menten parameter at the second temperature (c_{T2}).

Furthermore, in some embodiments, equations linear in temperature may be selected to relate the first, second, and third Michaelis-Menten parameters to temperature. For instance, in some embodiments, the first, second, and third Michaelis-Menten parameters may be written as

a_{T}=a₃₇*τₐ_{_{T}}(T)

b_{T}=b₃₇*τ_{b}_{_{T}}(T),

and

c_{T}=c₃₇*τ_{c}_{_{T}}(T) (Equation 2)

where τₐ_{_{T}}(T), τ_{b}_{_{T}}(T), and τ_{c}_{_{T}}(T) are “temperature correction factors” which approximately account for the temperature dependence of a_{T}, b_{T}, and c_{T}. When the relationship between Michaelis-Menten parameter and temperature is written as such, each Michaelis-Menten parameter a_{T}, b_{T}, and c_{T}, is determined by multiplying the 37° C. Michaelis-Menten parameter a₃₇, b₃₇, and c₃₇, by its corresponding “temperature correction factor,” τₐ_{_{T}}(T), τ_{b}_{_{T}}(T), or τ_{c}_{_{T}}(T), respectively. The 37° C. Michaelis-Menten parameters may be determined by fitting a modified Michaelis-Menten equation to a set of signals indicative of the analyte concentration of a plurality of solutions of differing analyte concentrations held at a temperature of 37° C., as described above with respect to, for example, T1 and T2. Alternatively, the parameters a₃₇, b₃₇, and c₃₇ may be supplied by a factory calibration as described in provisional U.S. Patent Publication No. 2010/0312483, which is hereby incorporated by reference in its entirety. As yet another alternative, a₃₇, b₃₇, and c₃₇ may be determined via a one-point in vivo calibration as also disclosed in the same application.

To determine the “temperature correction factors,” τₐ_{_{T}}(T), τ_{b}_{_{T}}(T), and τ_{c}_{_{T}}(T), some embodiment methods may employ a linear approximation. For instance, the temperature correction factors may be written as

τₐ_{_{T}}(T)=mₐ_{_{T}}*T+βₐ_{_{T}},

τ_{b}_{_{T}}(T)=m_{b}_{_{T}}*T+β_{b}_{_{T}},

and

τ_{c}_{_{T}}(T)=m_{c}_{_{T}}*T+β_{c}_{_{T}}, (Equation 3)

where the slopes, mₐ_{_{T}}, m_{b}_{_{T}}, m_{c}_{_{T}}, and intercepts, βₐ_{_{T}}, βb_{_{T}}, β_{c}_{_{T}}, are collectively referred to as “temperature calibration coefficients” (“TempCos”).

In some embodiments, a temperature calibration method used to determine values of these TempCos may require that values of the Michaelis-Menten parameters be determined at a second temperature (T2), different than 37° C. Values of the parameters at the second temperature (a_{T2}, b_{T2}, and c_{T2}) may be determined by fitting a modified Michaelis-Menten equation to a set of signals indicative of the analyte concentration of a plurality of solutions of differing analyte concentrations held at the second temperature, as described above with respect to, for example, T1 and T2. Once this is done, the temperature calibration coefficients mₐ and bₐ may be determined by normalizing to a₃₇ both a_{T2} and a₃₇, yielding a_{T2}/a₃₇ and 1, and fitting a line to the normalized values versus the two temperatures, T2 and 37° C. The fit may be determined using linear least squares or any other method of fitting a line to a set of points. The temperature calibration coefficient mₐ_{_{T}} is set equal to the slope of the resulting line and the temperature calibration coefficient βₐ_{_{T}} is set equal to the intercept. The other temperature calibration coefficients, m_{b}_{_{T}} and βₐ_{_{T}} may be determined similarly from values of b_{T2} and b₃₇, and m_{c}_{_{T}} and β_{c}_{_{T}} may be determined from values of c_{T2} and c₃₇. Once the calibration is complete, a temperature corrected estimated glucose concentration ([Glu]) may be computed from a fluorescent signal (Gᵢ) measured at temperature (T), by using the TempCos (mₐ_{_{T}}, βₐ_{_{T}}, m_{b}_{_{T}}, β_{b}_{_{T}}, m_{c}_{_{T}}, and β_{c}_{_{T}}), the 37° C. Michaelis-Menten parameters 37° C. (a₃₇, b₃₇, and c₃₇), and the temperature (T) in Equations 2 and 3 to compute a_{T}, b_{T}, and c_{T}, and then plugging a_{T}, b_{T}, c_{T} and the measured fluorescent signal (Gᵢ) into Equation 1.

Thus, in some embodiments the first, second, and third sets of temperature calibration parameters may include a slope and an intercept relating temperature to the value of either the first, second, or third Michaelis-Menten parameter. However, equations of other forms may be selected to relate the first, second, or third Michaelis-Menten equation to temperature. In some embodiments, a quadratic or higher-order polynomial in temperature may be suitable and/or desirable.

When measurement devices are mass produced, it may not be feasible or practical to individually calibrate each measurement device—i.e. use each individual measurement device to generate individual calibration data. It may be more cost effective to select one or more test devices from a batch of mass produced devices, generate calibration data using the one or more test devices, and provide that calibration data to each individual devices produced in the batch. In some embodiments, variability between measurement devices from the same production batch may be, to a large extent, attributable to a particular part of the measurement device. In particular, variability between devices may be attributable to the part of the measurement device which generates a signal indicative of analyte concentration—e.g. the analyte sensing element—and/or the part of the measurement device that generates a signal indicative of temperature—e.g. the temperature sensing element. In these circumstances, as well as others, it may be advantageous to use a calibration method employing multiple test measurement devices, and/or multiple test sensing elements, because calibration over multiple test devices and/or sensing elements may yield more accurate calibration data than calibration methods which only utilize a single test device and/or sensing element. Accordingly, in some embodiments, the calibration method may further include selecting a second test analyte sensing element; generating a third set of at least three signals using the second test analyte sensing element, each signal indicative of the concentration of analyte in a different solution of known analyte concentration at the first temperature (T1); and generating a fourth set of at least three signals using the second test analyte sensing element, each signal indicative of the concentration of analyte in a different solution of known analyte concentration at the second temperature (T2). Obviously, calibration methods may similarly employ more than two test devices, or more particularly, for instance, more than two test analyte sensing elements.

In a manner similar to methods utilizing a single test analyte sensing element, after the solutions having known analyte concentrations have been measured and the first, second, third, and fourth sets of at least three signals have been generated, in some embodiments, the sets of signals are used to determine (usually approximately) the relationship between one or more of the Michaelis-Menten parameters and temperature. For example, in some embodiments, the temperature calibration method may further include (1) computing values of each of a first, second, and third Michaelis-Menten parameter at the first temperature (a_{T1}, b_{T1}, and c_{T1}) by an algorithm comprising fitting a modified Michaelis-Menten equation to a first fit dataset comprising both the first set of at least three signals (which was generated with the first test analyte sensing element at T1) and the third set of at least three signals (which was generated with the second test analyte sensing element at T1); and (2) computing values of each of a first, second, and third Michaelis-Menten parameter at the second temperature (a_{T2}, b_{T2}, and c_{T2}) by an algorithm comprising fitting a modified Michaelis-Menten equation to a second fit dataset comprising both the second set of at least three signals (which was generated with the first test analyte sensing element at T2) and fourth set of at least three signals (which was generated with the second test analyte sensing element at T2). Essentially, in these types of methods, the signals generated with the second test analyte sensing element are used in a combined fit with the signals generated with the first test analyte sensing element, which results in values for each of the first, second, and third Michaelis-Menten parameters which take both test analyte sensing elements into account. Alternatively, in some embodiments, a temperature calibration method may take both test analyte sensing elements into account by fitting the signals generated from each test analyte sensing element separately, and then averaging the results to obtain better estimates of the Michaelis-Menten parameters. Thus, in some embodiments, the step of computing values of each of a first, second, and third Michaelis-Menten parameter at the first temperature (a_{T1}, b_{T1}, and c_{T1}) by an algorithm may further include fitting a modified Michaelis-Menten equation to a third fit dataset comprising the third set of at least three signals, and averaging the results of fitting the third fit dataset with the results of fitting the first fit dataset. In addition, the step of computing values of each of a first, second, and third Michaelis-Menten parameter at the second temperature (a_{T2}, b_{T2}, and c_{T2}) by an algorithm may further include fitting a modified Michaelis-Menten equation to a fourth fit dataset comprising the fourth set of at least three signals, and averaging the results of fitting the fourth fit dataset with the results of fitting the second fit dataset.

Other methods for estimating analyte concentration which incorporate temperature correction features and temperature calibration steps are also disclosed herein. In some embodiments, these methods are similar to those already described above and incorporate similar features, however, additional features may also be disclosed and, in some embodiments, the disclosed methods may be more general and described in more general terms. Since there are many ways to feasibly implement the discoveries disclosed herein for use in estimating analyte concentration, the following additional methods are described in order to illustrate the breadth of implementations that are possible.

In some embodiments, for instance, a method of estimating an analyte concentration from a signal indicative of the analyte concentration may include transforming the signal using an equation of the form of a modified Michaelis-Menten equation wherein the values of one or more Michaelis-Menten parameters have been adjusted for temperature.

In some embodiments, for instance, a method of estimating an analyte concentration may include generating a signal indicative of the analyte concentration and generating a signal indicative of a temperature, and transforming the signal indicative of the analyte concentration utilizing an equation of the form of a modified Michaelis-Menten equation wherein at least one of the Michaelis-Menten parameters has been substituted with a calibration equation functionally depending on a set of one or more temperature calibration parameters and the signal indicative of temperature. One could refer to such an equation as a “substituted” modified Michaelis-Menten equation since the Michaelis-Menten parameters have been explicitly substituted with equations depending on one or more other variables: temperature and the temperature calibration parameters. However, although such a “substituted” equation exhibits a more complicated analytic form, it nevertheless will still express the basic functional relationships of the modified Michaelis-Menten equation.

In some embodiments, the step of transforming the signal indicative of analyte concentration may utilize a “substituted” modified Michaelis-Menten equation in which each of the first, second, and third Michaelis-Menten parameters have been substituted with first, second, and third calibration equations (respectively), each of the equations depending on sets of first, second, and third temperature calibration parameters (respectively), and each also depending on the signal indicative of temperature. In certain embodiments, at least one of the first, second, and third calibration equations is a polynomial in the signal indicative of temperature. In certain such embodiments, each of the first, second, and third calibration equations is a polynomial in the signal indicative of temperature. In certain embodiments, at least one of the first, second, and third calibration equations is a linear equation in the signal indicative of temperature. In certain such embodiments, each of the first, second, and third calibration equations is a linear equation in the signal indicative of temperature. Thus, for example, if each Michaelis-Menten parameter of Equation 1 above is assumed to exhibit a linear relationship with temperature, then the “substituted” modified Michaelis-Menten equation might appear as

$$[ Glu ] = \frac{( {χ}_{{c}_{T} , 1} \cdot T + {χ}_{{c}_{T} , 0} ) * [ {G}_{i} - ( {χ}_{{a}_{T} , 1} \cdot T + {χ}_{{a}_{T} , 0} ) ]}{( {χ}_{{a}_{T} , 1} \cdot T + {χ}_{{a}_{T} , 0} ) + ( {χ}_{{b}_{T} , 1} \cdot T + {χ}_{{b}_{T} , 0} ) - {G}_{i}} \qquad ( Equation 4 )$$

and, similarly, if each Michaelis-Menten parameter is assumed to exhibit a quadratic relationship with temperature then the “substituted” modified Michaelis-Menten equation might appear as

$$[ Glu ] = \frac{\begin{matrix}( {χ}_{{c}_{T} , 2} \cdot {T}^{2} + {χ}_{{c}_{T} , 1} \cdot T + {χ}_{{c}_{T} , 0} ) * \\ [ {G}_{i} - ( {χ}_{{a}_{T} , 2} \cdot {T}^{2} + {χ}_{{a}_{T} , 1} \cdot T + {χ}_{{a}_{T} , 0} ) ]\end{matrix}}{\begin{matrix}( {χ}_{{a}_{T} , 2} \cdot {T}^{2} + {χ}_{{a}_{T} , 1} \cdot T + {χ}_{{a}_{T} , 0} ) + \\ ( {χ}_{{b}_{T} , 2} \cdot {T}^{2} + {χ}_{{b}_{T} , 1} \cdot T + {χ}_{{b}_{T} , 0} ) - {G}_{i}\end{matrix}} \qquad ( Equation 5 )$$

where:
[Glu] is the estimated glucose concentration,
χₐ_{_{T}}_{,2}, χₐ_{_{T}}_{,1}, and χₐ_{_{T}}_{,0} are polynomial coefficients parameterizing a_{T}'s dependence on the temperature T,
χ_{b}_{_{T}}_{,2}, χ_{b}_{_{T}}_{,1}, and χ_{b}_{_{T}}_{,0} are polynomial coefficients parameterizing b_{T}'s dependence on the temperature T,
χ_{c}_{_{T}}_{,2}, χ_{c}_{_{T}}_{,1}, and χ_{c}_{_{T}}_{,0} are polynomial coefficients parameterizing c_{T}'s dependence on the temperature T, and
Gᵢ is the fluorescent signal (i=1,2), either referenced or unreferenced, where G₁ is the fluorescence emission at 550 nm or 583 nm when the fluorophore is excited at 470 nm (which is the absorption maximum of the fluorophore's base-form), and G₂ is the fluorescence emission at 550 nm or 583 nm when the fluorophore is excited at 420 nm or 430 nm (which is the absorption maximum of the fluorophore's acid-form). Note, however, that other combinations of excitation and emission wavelengths are also feasible for use in Equations 4 and 5.

As stated above, although, the “substituted” equations (Equations 4 and 5) exhibit a more complicated analytic form, they nevertheless still exhibit the basic functional relationships of the modified Michaelis-Menten equation (Equation 1). In other embodiments, the calibration equations substituted into the modified Michaelis-Menten equation may have a functional form other than a polynomial in temperature.

Thus, as described above, the calibration equations substituted into the modified Michaelis-Menten equation for the Michaelis-Menten parameters may take a variety of functional forms and each may have varying numbers of temperature calibration parameters. Obviously, more complicated equations may have a greater numbers of temperature calibration parameters. In any event, depending on the embodiment, various temperature calibration methods may be used to determine the values of the first, second, and third sets of the one or more temperature calibration parameters. In certain such embodiments, each set of temperature calibration parameters may be determined by fitting the “substituted” modified Michaelis-Menten equation to a plurality of signals, the plurality of signals indicative of analyte concentration in a plurality of solutions at a plurality of temperatures. Once values of the various temperature calibration parameters are determined, temperature corrected estimates of analyte concentrations may be generated from signals indicative of analyte concentration and temperature.

An equation for computing a temperature corrected glucose concentration from a fluorescent signal and these temperature calibration parameters may be derived by substituting Equation 2 into Equation 1 which yields

$$[ Glu ] = \frac{{c}_{37} * {τ}_{c} ( T ) * [ {G}_{i} - {a}_{37} * {τ}_{a} ( T ) ]}{{a}_{37} * {τ}_{a} ( T ) + {b}_{37} * {τ}_{b} ( T ) - {G}_{i}} \qquad ( Equation 6 )$$

and then, using Equation 3, further substituting for τₐ(T), τ_{b}(T), and τ_{c}(T) in Equation (6), which yields

$$[ Glu ] = \frac{{c}_{37} * ( {m}_{c} * T + {b}_{c} ) * [ {G}_{i} - {a}_{37} * ( {m}_{a} * T + {b}_{a} ) ]}{{a}_{37} * ( {m}_{a} * T + {b}_{a} ) + {b}_{37} * ( {m}_{b} * T + {b}_{b} ) - {G}_{i}} \qquad ( Equation 7 )$$

### Methods of Measuring pH

If a solution's measured analyte concentration is to be corrected for pH effects, the pH of the solution must be measured or estimated in some manner. In some embodiments a separate pH sensor may be used to measure pH. In other embodiments, the same indicator system which is used to generate a signal indicative of analyte concentration may be used to measure pH. For instance, the ratio of two green signals generated by the indicator system may be used to compute pH through the following relationship:

$$pH = {m}_{pH} * \frac{{G}_{1}}{{G}_{2}} + {β}_{pH } \qquad ( Equation 8 )$$

where m_{pH} is the slope and β_{pH} is the intercept of pH versus G₁/G₂. The ratio, G₁/G₂, is calculated from G₁ which is the fluorescence emission at 550 nm or 583 nm when the fluorophore is excited at 470 nm, and G₂ which is the fluorescence emission at 550 nm or 583 nm when the fluorophore is excited at 420 nm or 430 nm. The relationship between G₁/G₂ and pH is approximate linear over a range of glucose concentrations (50 mg/dL, 100 mg/dL, 200 mg/dL, and 400 mg/dL) and pH levels (6.8, 7.2, 7.4, and 7.8), although some greater deviation from linearity occurs between pH 7.4 and pH 7.8. Note, that G₁/G₂ is represented as Ibase/Iacid since, as indicated above, 470 nm is the absorption maximum of the fluorophore's base-form, and 430 nm is the absorption maximum of the fluorophore's acid-form. Also, note that the values of G₁/G₂ can be normalized to the 100 mg/dL, pH 7.4 value of G₁/G₂. Thus, Equation 8 may be used to predict pH level from the G₁/G₂ ratio once the constants m_{pH} and β_{pH} have been determined. In some embodiments, each analyte measurement device may be individually calibrated to determine the constants m_{pH} and β_{pH} appropriate for that individual device. In other embodiments, an entire batch of measurement devices may be calibrated by selecting several devices from the batch, determining values of m_{pH} and β_{pH} for each selected device, and averaging the values of m_{pH} and β_{pH} obtained for each selected devices to produce averaged values of m_{pH} and β_{pH} valid for the entire batch of measurement devices for use with Equation 8. In still other embodiments, averaged values of m_{pH} and β_{pH} may be determined as just described, but a one-point calibration is performed to individually calibrate each sensor in the batch while taking advantage of the averaged values of m_{pH} and β_{pH} obtained for the entire batch. For instance, in some embodiments, the one point calibration performed on each individual measuring device may involve using the individual device to measure G₁ and G₂ for a standard solution having a glucose concentration of 100 mg/dL at pH 7.4. These values may then be used in Equation 9

$$pH = {m}_{pH} * \frac{\frac{{G}_{1}}{{G}_{2}}}{\frac{{G}_{1 , 7.4}}{{G}_{2 , 7.4}}} + {β}_{pH } \qquad ( Equation 9 )$$

where:
G₁ is the fluorescent emission, either referenced or unreferenced, at 550 nm or 583 nm when the fluorophore is excited at 470 nm, which is the absorption maximum of the fluorophore's base-form (although other combinations of excitation and emission wavelengths are also feasible for use as the numerator of the G₁/G₂ ratio in Equation 9),
G₂ is the fluorescence emission, either referenced or unreferenced, at 550 nm or 583 nm when the fluorophore is excited at 430 nm, which is the absorption maximum of the fluorophore's acid-form (although other combinations of excitation and emission wavelengths are also feasible for use as the denominator of the G₁/G₂ ratio in Equation 9),
G_{1,7.4}=G₁ signal at pH 7.4 and 100 mg/dL glucose concentration, G_{2,7.4}=G₂ signal at pH 7.4 and 100 mg/dL glucose concentration,
m_{pH}=pH slope, and
β_{pH}=pH intercept.

Thus, once universal values of m_{pH} and β_{pH} are determined for the batch of measurement devices, and G_{1,7.4} and G_{2,7.4} are determined via one-point calibration for the individual measurement device, a measured ratio G₁/G₂ may be used in Equation 9 to compute the pH level of the solution of analyte. It was also discovered that the determination of pH from G₁/G₂ is effected by the temperature of the solution. Moreover, for purposes of estimating pH from the measured ratio G₁/G₂, the temperature dependence may be taken into account by allowing m_{pH} and β_{pH} to vary with temperature. In particular, the temperature dependence of m_{pH} and β_{pH} may be modeled using Equations 10 and 11:

$$\begin{matrix}{m}_{pH} = \frac{h}{T} + i & ( Equation 10 ) \\ {β}_{pH } = j * \sqrt{T} + k & ( Equation 11 )\end{matrix}$$

where h, i, j, and k are empirically determined. Substituting Equations 10 and 11 into Equation 9 gives an expression for computing pH from the ratio G₁/G₂ which accurately, albeit approximately, takes temperature into account.

$$pH = ( \frac{h}{T} + i ) * \frac{\frac{{G}_{1}}{{G}_{2}}}{\frac{{G}_{1 , 7.4}}{{G}_{2 , 7.4}}} + j * \sqrt{T} + k \qquad ( Equation 12 )$$

### Methods of Estimating Analyte Concentration Incorporating pH Correction

Some embodiments of the measurement devices disclosed herein generate a signal indicative of analyte concentration which exhibits a pH dependence. For example, if two solutions of precisely the same analyte concentration are measured at two different pH levels with the same measurement device, in some embodiments, the measurement device may generate differing signals indicative of the two analyte concentrations. Thus, the accuracy of determining a solution's true analyte concentration based on such as signal may be improved by taking the pH of the solution into account.

It has been discovered that for some embodiments of the measurement devices disclosed herein, and in particular, for glucose measurement devices employing a quencher binding moiety operably coupled to a fluorophore, the pH dependence of the fluorescent signal approximately follows a modified version of the classic Michaelis-Menten equation from enzyme kinetics:

$$[ Glu ] = \frac{{c}_{pH} * ⌊ {G}_{i} - {a}_{pH} ⌋}{{a}_{pH} + {b}_{pH} - {G}_{i}} \qquad ( Equation 13 )$$

where:
[Glu] is the estimated glucose concentration,
a_{pH} is the first Michaelis-Menten parameter “a”, at a particular pH,
b_{pH} is the second Michaelis-Menten parameter “b”, at the same particular pH,
c_{pH} is the third Michaelis-Menten parameter “c”, at the same particular pH, and
Gᵢ is the fluorescent signal (i=1,2), either referenced or unreferenced, where G₁ is the fluorescence emission at 550 nm or 583 nm when the fluorophore is excited at 470 nm (which is the absorption maximum of the fluorophore's base-form), and G₂ is the fluorescence emission at 550 nm or 583 nm when the fluorophore is excited at 430 nm (which is the absorption maximum of the fluorophore's acid-form). Note, however, that other combinations of excitation and emission wavelengths are also feasible for use in Equation 13.

As with temperature dependence, the fact that pH dependence may be described by a modified Michaelis-Menten equation is an interesting and surprising result. Various embodiments of the measurement devices disclosed herein employ a quencher-fluorophore indicator system which measures analyte concentration through the establishment of an equilibrium between the analyte of interest, the binding moiety (e.g. quencher), and the fluorophore. In such a system, analyte concentration is not measured by enzymatic consumption or conversion of the analyte. In contrast, the classic Michaelis-Menten equation specifically describes enzyme kinetics, a non-equilibrium phenomena involving the consumption/conversion of the enzyme's substrate by the enzyme. Therefore, it is not to be expected that an equation closely related to the classic Michaelis-Menten equation would effectively describe the pH dependence of these types of quencher-fluorophore-based measurement devices and analyte sensing elements (or other measurement devices and analyte sensing elements functioning through analogous equilibrium mechanisms). In any event, knowledge that these devices (and similar devices) exhibit a pH dependence which follows a modified Michaelis-Menten equation allows the use of pH correction methods and algorithms to improve the accuracy of analyte concentration measurements. Such methods and algorithms are disclosed herein, along with measurement devices which implement such methods and algorithms.

Accordingly, some embodiment methods of estimating an analyte concentration include generating a signal indicative of analyte concentration and a signal indicative of pH. In some embodiments, the signal indicative of the analyte concentration and the signal indicative of the pH are both generated from a set of at least two signals each of which is indicative of both the pH and the analyte concentration. For instance, these could be the G₁ and G₂ fluorescent signals described above, both of which are indicative of both analyte concentration and pH. In some embodiments, the G₂ fluorescent signal itself may be treated as the signal indicative of analyte concentration, while G₁ and G₂ are used as signals indicative of pH, for example, in the pH determination algorithm described above. Since, in some embodiments, the signal indicative of analyte concentration exhibits a pH dependence, in some embodiments, the signal indicative of pH may be used to adjust the signal indicative of analyte concentration to correct for pH dependence. Thus, in certain such embodiments, the methods further include transforming the signal indicative of the analyte concentration utilizing an equation of the form of a modified Michaelis-Menten equation, such as Equation 1 above, depending on Michaelis-Menten parameters, such as the parameters “a”, “b”, and “c”, as described above as first, second, and third Michaelis-Menten parameters with reference to Equation 13.

Of course, it is to be understood that when a signal is described herein as being indicative of one physical quantity, such description is not meant to necessarily preclude that signal from also being indicative of another physical quantity. For instance, a computed analyte concentration that may be improved through pH correction, was likely computed from a signal indicative of analyte concentration which contained some pH dependency. Therefore, to a certain extent, such a signal indicative of analyte concentration may also be considered a signal indicative of pH, as will be readily appreciated by one of skill in the art.

The pH dependence of Equation 13 is exhibited through the Michaelis-Menten parameters a_{pH}, b_{pH}, and c_{pH}, as indicated by the subscript “pH” labeling these parameters. In some embodiments, the pH dependence may need to be determined through a pH calibration. Thus, in certain embodiment methods, the values of one or more of the Michaelis-Menten parameters may be set based on data which includes pH calibration data and the signal indicative of a pH.

For example, in some embodiment methods, the pH calibration data may be generated by a pH calibration method. The pH calibration method may include selecting a first test analyte sensing element, and creating and/or providing a set of at least three solutions of differing known analyte concentrations. In certain such embodiments, a first pH is selected (pH1), three solutions of the set of at least three solutions are adjusted to a pH substantially similar to the selected first pH, and a first set of at least three signals is generated using the first test analyte sensing element, each signal indicative of the concentration of analyte in a different one of the three solutions at the first pH. Measurements are then made at a second pH. Thus, in certain embodiments, a second pH is selected (pH2), three solutions of the set of at least three solutions (each of the three may be the same or different than a solution chosen for the first pH) are adjusted to a pH substantially similar to the selected second pH, and a second set of at least three signals is generated using the first test analyte sensing element, each signal indicative of the concentration of analyte in a different one of the three solutions at the second pH. Of course, more than three solutions may be used in either of these steps. And more than two pHs may also be employed. Generally, the more solutions of differing concentration and the greater number of different pHs that are employed, the greater the accuracy of the resulting calibration data.

Once the solutions having known analyte concentrations have been measured, and the first and second sets of at least three signals have been generated, in some embodiments, the sets of signals are used to determine (usually approximately) the relationship between one or more of the Michaelis-Menten parameters and pH. For example, in some embodiments, the pH calibration method may further include computing values of each of a first, second, and third Michaelis-Menten parameter at the first pH (a_{pH1}, b_{pH1}, and c_{pH1}) by an algorithm comprising fitting a modified Michaelis-Menten equation to a first fit dataset comprising the first set of at least three signals. In certain such embodiments, the pH calibration method may further include computing values of each of a first, second, and third Michaelis-Menten parameter at the second pH (a_{pH2}, b_{pH2}, and c_{pH2}) by an algorithm comprising fitting a modified Michaelis-Menten equation to a second fit dataset comprising the second set of at least three signals. Thus, in methods such as these, each of the three Michaelis-Menten parameters has been determined at least two pHs, providing data which may be used to create a model of the pH dependence of each of the three Michaelis-Menten parameters.

To model the pH dependence of the Michaelis-Menten parameters, in some embodiments, the pH calibration method may further include selecting an equation relating the first Michaelis-Menten parameter (a_{pH}) to pH, the equation depending on a first set of pH calibration parameters; and setting a value for each calibration parameter of the first set of calibration parameters based on the value of the first Michaelis-Menten parameter at the first pH (a_{pH1}) and the value of the first Michaelis-Menten parameter at the second pH (a_{pH2}). In some embodiments, similar steps are performed with respect to the second and third Michaelis-Menten parameters (b_{pH} and c_{pH}). Thus, for example, the pH calibration method may further include selecting an equation relating the second Michaelis-Menten parameter (b_{pH}) to pH, the equation depending on a second set of pH calibration parameters; and setting a value for each calibration parameter of the second set of calibration parameters based on the value of the second Michaelis-Menten parameter at the first pH (b_{pH1}) and the value of the second Michaelis-Menten parameter at the second pH (b_{pH2}). Similarly, in some embodiments, the pH calibration method may further include selecting an equation relating the third Michaelis-Menten parameter (c_{pH}) to pH, the equation depending on a third set of pH calibration parameters; and setting a value for each calibration parameter of the third set of calibration parameters based on the value of the third Michaelis-Menten parameter at the first pH (c_{pH1}) and the value of the third Michaelis-Menten parameter at the second pH (c_{pH2}).

Furthermore, in some embodiments, equations linear in pH may be selected to relate the first and second Michaelis-Menten parameters to pH, while a more complicated equation may be selected to relate the third Michaelis-Menten parameter to pH. For instance, in some embodiments, the first, second, and third Michaelis-Menten parameters may be written as

a_{pH}=a_{7.4}*ρₐ_{_{pH}}(pH),

b_{pH}=b_{7.4}*ρ_{b}_{_{pH}}(pH),

and

c_{pH}=c_{7.4}*ρ_{c}_{_{pH}}(pH) (Equation 14)

where ρₐ_{_{pH}}(pH), ρ_{b}_{_{pH}}(pH), and ρ_{c}_{_{pH}}(pH) are “pH correction factors” which approximately account for the pH dependence of a_{pH}, b_{pH}, and c_{pH}. When the relationship between Michaelis-Menten parameter and pH is written as such, each Michaelis-Menten parameter a_{pH}, b_{pH}, and c_{pH}, is determined by multiplying the pH 7.4 Michaelis-Menten parameter a_{7.4}, b_{7.4}, and c_{7.4}, by its corresponding “pH correction factor,” ρₐ_{_{pH}}(pH), ρ_{b}_{_{pH}}(pH), or ρ_{c}_{_{pH}}(pH), respectively. The pH 7.4 Michaelis-Menten parameters may be determined by fitting a modified Michaelis-Menten equation to a set of signals indicative of the analyte concentration of a plurality of solutions of differing analyte concentrations held at pH 7.4, as described above with respect to, for example, pH1 and pH2. Alternatively, the parameters a_{7.4}, b_{7.4}, and c_{7.4} may be supplied by a factory calibration as described in provisional U.S. Patent Publication No. 2010/0312483, which is hereby incorporated herein by reference in its entirety. As yet another alternative, a_{7.4}, b_{7.4}, and c_{7.4} may be determine via a one-point in vivo calibration as also disclosed in the same application.

To determine the “pH correction factors,” ρₐ_{_{pH}}(pH), ρ_{b}_{_{pH}}(pH), ρ_{c}_{_{pH}}(pH), some embodiment methods may select a first equation linear in pH to relate the first Michaelis-Menten parameter to pH, and select a second equation linear in pH to relate the second Michaelis-Menten parameter to pH. In certain such embodiment methods, an equation is selected to relate the third Michaelis-Menten parameter to pH which comprises a fraction wherein the numerator is equal to an exponential function of an equation linear in the inverse of pH, and the denominator is equal to an exponential function of the same linear function in the inverse of pH evaluated at pH 7.4. If such equations in pH are selected, then the pH correction factors may be written as

$${ρ}_{{a}_{pH}} ( pH ) = {m}_{{a}_{pH}} * pH + {β}_{{a}_{pH}} , \\ {ρ}_{{b}_{pH}} ( pH ) = {m}_{{b}_{pH}} * pH + {β}_{{b}_{pH}} , and \\ {ρ}_{{c}_{pH}} ( pH ) = \frac{{e}^{( \frac{{m}_{{c}_{pH}}}{pH} + {β}_{{c}_{pH}} )}}{{e}^{( \frac{{m}_{{c}_{pH}}}{7.4} + {β}_{{c}_{pH}} )}} . \qquad ( Equation 15 )$$

where the slopes, mₐ_{_{pH}}, m_{b}_{_{pH}}, m_{c}_{_{pH}}, and intercepts, βₐ_{_{pH}}, β_{b}_{_{pH}}, β_{c}_{_{pH}}, are collectively referred to as pH calibration coefficients (“pHCos”). However, analytic functional forms other than linear equations may be chosen to relate the pH correction factors and/or Michaelis-Menten parameters to pH (or to inverses of pH as indicated by Equation 15's expression for ρ_{c}_{_{pH}}(pH)). For instance, in some embodiments, quadratic or higher-order polynomials in pH may be appropriate and/or desirable.

In various embodiments, a pH calibration method used to determine values of these pHCos may require that values of the Michaelis-Menten parameters be determined at a second pH (pH2), different than pH 7.4. Values of the parameters at the second pH (a_{pH2}, b_{pH2}, and c_{pH2}) may be determined by fitting a modified Michaelis-Menten equation to a set of signals indicative of the analyte concentration of a plurality of solutions of differing analyte concentrations held at the second pH, as described above with respect to, for example, pH1 and pH2. Once this is done, the pH calibration coefficients mₐ_{_{pH}} and βₐ_{_{pH}} may be determined by normalizing to a_{7.4} both a_{pH2} and a_{7.4}, yielding a_{pH2}/a_{7.4} and 1, and fitting a line to the normalized values versus the two pHs, pH2 and pH 7.4. The fit may be determined using linear least squares or any other method of fitting a line to a set of points. The pH calibration coefficient, mₐ_{_{pH}}, is set equal to the slope of the resulting line and the pH calibration coefficient, βₐ_{_{pH}}, is set equal to the intercept. The pH calibration coefficients m_{b}_{_{pH}} and β_{b}_{_{pH}} may be determined the same way from values of b_{pH2} and b_{7.4}. Finally, in a manner analogous to the determination of mₐ_{_{pH}}, βₐ_{_{pH}}, m_{b}_{_{pH}}, and β_{b}_{_{pH}}, the pH calibration coefficients m_{c}_{_{pH}} and β_{c}_{_{pH}} may be determined from values of c_{pH2} and c_{7.4}, however an additional step of linearizing Equation 15's expression for ρ_{c}_{_{pH}}(pH) must first be performed. Once the calibration is complete, a pH corrected estimated glucose concentration ([Glu]) may be (computed from a fluorescent signal (Gᵢ) measured at a particular pH, by using the pHCos (mₐ_{_{pH}}, βₐ_{_{pH}}, m_{b}_{_{pH}}, β_{b}_{_{pH}}, m_{c}_{_{pH}}, and β_{c}_{_{pH}}), the pH 7.4 Michaelis-Menten parameters (a_{7.4}, b_{7.4}, and c_{7.4}), and the measured pH (pH) in Equations 14 and 15 to compute a_{pH}, and c_{pH}, and then plugging a_{pH}, b_{pH}, c_{pH} and the measured fluorescent signal (Gᵢ) into Equation 13.

Thus, in some embodiments, the first set of pH calibration parameters comprises the slope and intercept of a first equation linear in pH, and in some embodiments, the second set of pH calibration parameters comprises the slope and intercept of a second equation linear in pH. In certain such embodiments, the third set of pH calibration parameters may comprise the slope and intercept of an equation linear in the inverse of pH which is related to the third Michaelis-Menten parameter through an exponential function divided by a constant—wherein the constant is equal to the result of evaluating the exponential function of the same equation linear in the inverse of pH evaluated at a fixed pH level. However, the pH calibration parameters (pHCos) may comprise constants associated with analytic functional forms other than linear equations which may be suitable and/or desirable. For instance, in some embodiments, the pHCos may include the coefficients of quadratic or higher-order polynomials in pH.

When measurement devices are mass produced, it may not be feasible or practical to individually calibrate each measurement device—i.e. use each individual measurement device to generate individual calibration data. It may be more cost effective to select one or more test devices from a batch of mass produced devices, generate calibration data using the one or more test devices, and provide that calibration data to each individual devices produced in the batch. In some embodiments, variability between measurement devices from the same production batch may be, to a large extent, attributable to a particular part of the measurement device. In particular, variability between devices may be attributable to the part of the measurement device which generates a signal indicative of analyte concentration—e.g. the analyte sensing element—and/or the part of the measurement device that generates a signal indicative of pH—e.g. the pH sensing element. In these circumstances, as well as others, it may be advantageous to use a calibration method employing multiple test measurement devices, and/or multiple test sensing elements, because calibration over multiple test devices and/or sensing elements may yield more accurate calibration data than calibration methods which only utilize a single test device and/or sensing element. Accordingly, in some embodiments, the calibration method may further include selecting a second test analyte sensing element; generating a third set of at least three signals using the second test analyte sensing element, each signal indicative of the concentration of analyte in a different solution of known analyte concentration at the first pH (pH1); and generating a fourth set of at least three signals using the second test analyte sensing element, each signal indicative of the concentration of analyte in a different solution of known analyte concentration at the second pH (pH2). Obviously, calibration methods may similarly employ more than two test devices, or more particularly, for instance, more than two test analyte sensing elements.

In a manner similar to methods utilizing a single test analyte sensing element, after the solutions having known analyte concentrations have been measured and the first, second, third, and fourth sets of at least three signals have been generated, in some embodiments, the sets of signals are used to determine (usually approximately) the relationship between one or more of the Michaelis-Menten parameters and pH. For example, in some embodiments, the pH calibration method may further include (1) computing values of each of a first, second, and third Michaelis-Menten parameter at the first pH (a_{pH1}, b_{pH1}, and c_{pH1}) by an algorithm comprising fitting a modified Michaelis-Menten equation to a first fit dataset comprising both the first set of at least three signals (which was generated with the first test analyte sensing element at pH1) and the third set of at least three signals (which was generated with the second test analyte sensing element at pH1); and (2) computing values of each of a first, second, and third Michaelis-Menten parameter at the second pH (a_{pH2}, b_{pH2}, and c_{pH2}) by an algorithm comprising fitting a modified Michaelis-Menten equation to a second fit dataset comprising both the second set of at least three signals (which was generated with the first test analyte sensing element at pH2) and fourth set of at least three signals (which was generated with the second test analyte sensing element at pH2). Essentially, in these types of methods, the signals generated with the second test analyte sensing element are used in a combined fit with the signals generated with the first test analyte sensing element, which results in values for each of the first, second, and third Michaelis-Menten parameters which take both test analyte sensing elements into account. Alternatively, in some embodiments, a pH calibration method may take both test analyte sensing elements into account by fitting the signals generated from each test analyte sensing element separately, and then averaging the results to obtain better estimates of the Michaelis-Menten parameters. Thus, in some embodiments, the step of computing values of each of a first, second, and third Michaelis-Menten parameter at the first pH (a_{pH1}, b_{pH1}, and c_{pH1}) by an algorithm may further include fitting a modified Michaelis-Menten equation to a third fit dataset comprising the third set of at least three signals, and averaging the results of fitting the third fit dataset with the results of fitting the first fit dataset. In addition, the step of computing values of each of a first, second, and third Michaelis-Menten parameter at the second pH (a_{pH2}, b_{pH2}, and c_{pH2}) by an algorithm may further include fitting a modified Michaelis-Menten equation to a fourth fit dataset comprising the fourth set of at least three signals, and averaging the results of fitting the fourth fit dataset with the results of fitting the second fit dataset.

Other methods for estimating analyte concentration which incorporate pH correction features and pH calibration steps are also disclosed herein. In some embodiments, these methods are similar to those already described above and incorporate similar features, however, additional features may also be disclosed and, in some embodiments, the disclosed methods may be more general and described in more general terms. Since there are many ways to feasibly implement the discoveries disclosed herein for use in estimating analyte concentration, the following additional methods are described in order to illustrate the breadth of implementations that are possible.

In some embodiments, for instance, a method of estimating an analyte concentration from a signal indicative of the analyte concentration may include transforming the signal using an equation of the form of a modified Michaelis-Menten equation wherein the values of one or more Michaelis-Menten parameters have been adjusted for pH.

In some embodiments, for instance, a method of estimating an analyte concentration may include generating a signal indicative of the analyte concentration and generating a signal indicative of a pH, and transforming the signal indicative of the analyte concentration utilizing an equation of the form of a modified Michaelis-Menten equation wherein at least one of the Michaelis-Menten parameters has been substituted with a calibration equation functionally depending on a set of one or more pH calibration parameters and the signal indicative of pH. One could refer to such an equation as a “substituted” modified Michaelis-Menten equation since the Michaelis-Menten parameters have been explicitly substituted with equations depending on one or more other variables—pH and the pH calibration parameters. However, although such a “substituted” equation exhibits a more complicated analytic form, it nevertheless will still express the basic functional relationships of the modified Michaelis-Menten equation.

In some embodiments, the step of transforming the signal indicative of analyte concentration may utilize a “substituted” modified Michaelis-Menten equation in which each of the first, second, and third Michaelis-Menten parameters have been substituted with first, second, and third calibration equations (respectively), each of the equations depending on sets of first, second, and third pH calibration parameters (respectively), and each also depending on the signal indicative of pH. In certain embodiments, at least one of the first, second, and third calibration equations is a polynomial in the signal indicative of pH. In certain such embodiments, each of the first, second and third calibration equations is a polynomial in the signal indicative of pH. In certain embodiments, at least one of the first, second, and third calibration equations is a linear equation in the signal indicative of pH. In certain such embodiments, the first and second calibration equations are a linear equations in the signal indicative of pH, and the third calibration equation comprises a fraction wherein the numerator is equal to an exponential function of an equation linear in the inverse of the signal indicative of pH, and the denominator is equal to an exponential function of the same linear function in the inverse of the signal indicative of pH evaluated at fixed pH.

Thus, for example, if each Michaelis-Menten parameter of Equation 13 above is assumed to exhibit a linear relationship with pH, then the “substituted” modified Michaelis-Menten equation might appear as

$$[ Glu ] = \frac{\begin{matrix}( {χ}_{{c}_{pH} , 1} \cdot pH + {χ}_{{c}_{pH} , 0} ) * \\ [ {G}_{i} - ( {χ}_{{a}_{pH} , 1} \cdot pH + {χ}_{{a}_{pH} , 0} ) ]\end{matrix}}{\begin{matrix}( {χ}_{{a}_{pH} , 1} \cdot pH + {χ}_{{a}_{pH} , 0} ) + \\ ( {χ}_{{b}_{pH} , 1} \cdot pH + {χ}_{{b}_{pH} , 0} ) - {G}_{i}\end{matrix}} \qquad ( Equation 16 )$$

and, similarly, if each Michaelis-Menten parameter is assumed to exhibit a quadratic relationship with pH then the “substituted” modified Michaelis-Menten equation might appear as

$$[ Glu ] = \frac{\begin{matrix}( {χ}_{{c}_{pH} , 2} \cdot {pH}^{2} + {χ}_{{c}_{pH} , 1} \cdot pH + {χ}_{{c}_{pH} , 0} ) * \\ [ {G}_{i} - ( {χ}_{{a}_{pH} , 2} \cdot {pH}^{2} + {χ}_{{a}_{pH} , 1} \cdot pH + {χ}_{{a}_{pH} , 0} ) ]\end{matrix}}{\begin{matrix}( {χ}_{{a}_{pH} , 2} \cdot {pH}^{2} + {χ}_{{a}_{pH} , 1} \cdot pH + {χ}_{{a}_{pH} , 0} ) + \\ ( {χ}_{{b}_{pH} , 2} \cdot {pH}^{2} + {χ}_{{b}_{pH} , 1} \cdot pH + {χ}_{{b}_{pH} , 0} ) - {G}_{i}\end{matrix}} \qquad ( Equation 17 )$$

where:
[Glu] is the estimated glucose concentration,
χₐ_{_{pH}}_{,2}, χₐ_{_{pH}}_{,1}, and χₐ_{_{pH}}_{,0} are polynomial coefficients parameterizing a_{pH}'s dependence on the pH level,
χ_{b}_{_{pH}}_{,2}, χ_{b}_{_{pH}}_{,1} and χ_{b}_{_{pH}}_{,0} are polynomial coefficients parameterizing b_{pH}'s dependence on the pH level,
χ_{c}_{_{pH}}_{,2}, χ_{c}_{_{pH}}_{,1}, and χ_{c}_{_{pH}}_{,0} are polynomial coefficients parameterizing c_{pH}'s dependence on the pH level, and
Gᵢ is the fluorescent signal (i=1,2), either referenced or unreferenced, where G₁ is the fluorescence emission at 550 nm or 583 nm when the fluorophore is excited at 470 nm (which is the absorption maximum of the fluorophore's base-form), and G₂ is the fluorescence emission at 550 nm or 583 nm when the fluorophore is excited at 430 nm (which is the absorption maximum of the fluorophore's acid-form). Note, however, that other combinations of excitation and emission wavelengths are also feasible for use in Equations 16 and 17.

As stated above, although, the “substituted” equations (Equations 16 and 17) exhibit a more complicated analytic form, they nevertheless still exhibit the basic functional relationships of the modified Michaelis-Menten equation (Equation 13). In other embodiments, the calibration equations substituted into the modified Michaelis-Menten equation may have a functional form other than a polynomial in pH.

Thus, as described above, the calibration equations substituted into the modified Michaelis-Menten equation for the Michaelis-Menten parameters may take a variety of functional forms and each may have varying numbers of pH calibration parameters. Obviously, more complicated equations may have a greater numbers of pH calibration parameters. In any event, depending on the embodiment, various pH calibration methods may be used to determine the values of the first, second and third sets of the one or more pH calibration parameters. In certain such embodiments, each set of pH calibration parameters may be determined by fitting the “substituted” modified Michaelis-Menten equation to a plurality of signals, the plurality of signals indicative of analyte concentration in a plurality of solutions at a plurality of pHs. Once values of the various pH calibration parameters are determined, pH corrected estimates of analyte concentrations may be generated from signals indicative of analyte concentration and pH.

### Temperature and pH Correction

Furthermore, in certain embodiments, the analyte sensors further include a receiving and processing unit configured to transform the signal indicative of the analyte concentration based, in part, on a signal indicative of temperature or pH generated by the temperature or pH sensing element. In certain embodiments, the receiving and processing unit is configured to transform the signal indicative of the analyte concentration utilizing an equation of the form of a modified Michaelis-Menten equation depending on Michaelis-Menten parameters. In some embodiments, the values of the Michaelis-Menten parameters are set based on data comprising temperature calibration data and the signal indicative of the temperature, as well as pH calibration data and signal indicative of the pH. Such calibration and correction methods are described in greater detail in co-pending U.S. application Ser. No. 13/046,571; incorporated herein in its entirety by reference.

Some embodiments of the analyte sensors disclosed herein generate a signal indicative of analyte concentration which exhibits a temperature dependence. For example, if two solutions of precisely the same analyte concentration are measured at two different temperatures with the same analyte sensor, in some embodiments, the analyte sensor may generate differing signals indicative of the two analyte concentrations. Thus, the accuracy of determining a solution's true analyte concentration based on such as signal may be improved by taking the temperature of the solution into account.

It has been discovered that for some embodiments of the analyte sensors disclosed herein, and in particular, for glucose sensors employing a quencher binding moiety operably coupled to a fluorophore, the temperature dependence of the fluorescent signal approximately follows a modified version of the classic Michaelis-Menten equation from enzyme kinetics:

$$[ Glu ] = \frac{{c}_{T} * [ F - {a}_{T} ]}{{a}_{T} + {b}_{T} - F}$$

where
[Glu] is the estimated glucose concentration
F is the fluorescent signal,
a_{T} is the Michaelis-Menten parameter “a”, at a temperature T,
b_{T} is the Michaelis-Menten parameter “b”, at the same temperature T, and
c_{T} is the Michaelis-Menten parameter “c”, at the same temperature T.

In itself, this is an interesting and surprising result. Various embodiments of the analyte sensors disclosed herein employ a quencher-fluorophore indicator system which measures analyte concentration through the establishment of an equilibrium between the analyte of interest, the binding moiety (e.g. quencher), and the fluorophore. In such a system, analyte concentration is not measured by enzymatic consumption or conversion of the analyte. In contrast, the classic Michaelis-Menten equation specifically describes enzyme kinetics, a non-equilibrium phenomena involving the consumption/conversion of the enzyme's substrate by the enzyme. Therefore, it is not to be expected that an equation closely related to the classic Michaelis-Menten equation would effectively describe the temperature dependence of these types of quencher-fluorophore based sensors (or other sensors functioning through analogous equilibrium mechanisms). In any event, knowledge that these sensors (and similar sensors) exhibit a temperature dependence which follows a modified Michaelis-Menten equation allows the use of temperature correction methods and algorithms to improve the accuracy of analyte concentration measurements. Such methods and algorithms are disclosed herein, along with devices which implement such methods and algorithms.

Accordingly, some embodiment methods of estimating an analyte concentration include generating a signal indicative of analyte concentration and a signal indicative of temperature using an analyte sensor. Since, in some embodiments, the signal indicative of analyte concentration exhibits the temperature dependence just described, in some embodiments, the signal indicative of temperature may be used to adjust the signal indicative of analyte concentration to correct for temperature dependence. Thus, in certain such embodiments, the methods further include transforming the signal indicative of the analyte concentration utilizing an equation of the form of a modified Michaelis-Menten equation, depending on Michaelis-Menten parameters, such as the parameters “a”, “b”, and “c”, as described above.

The temperature dependence is exhibited through the Michaelis-Menten parameters a_{T}, b_{T}, and c_{T}, as indicated by the subscript “T” labeling these parameters. In some embodiments, the temperature dependence may need to be determined through a temperature calibration. Thus, in certain embodiment methods, the values of one or more of the Michaelis-Menten parameters may be set based on data which includes temperature calibration data and the signal indicative of a temperature.

For example, in some embodiment methods, the temperature calibration data may be generated by a temperature calibration method. The temperature calibration method may include selecting a first analyte test sensor, and creating and/or providing a set of at least three solutions of differing known analyte concentrations. In certain such embodiments, a first temperature is selected (T1), three solutions of the set of at least three solutions are heated and/or cooled to a temperature substantially similar to the selected first temperature, and a first set of at least three signals is generated using the first analyte test sensor, each signal indicative of the concentration of analyte in a different one of the three solutions at the first temperature. Measurements are then made at a second temperature. Thus, in certain embodiments, a second temperature is selected (T2), three solutions of the set of at least three solutions (each of the three may be the same or different than a solution chosen for the first temperature) are heated and/or cooled to a temperature substantially similar to the selected second temperature, and a second set of at least three signals is generated using the first analyte test sensor, each signal indicative of the concentration of analyte in a different one of the three solutions at the second temperature. Of course, more than three solutions may be used in either of these steps. And more than two temperatures may also be employed. Generally, the more solutions of differing concentration and the greater number of different temperatures that are employed, the greater the accuracy of the resulting calibration data.

Once the solutions having known analyte concentrations have been measured, and the first and second sets of at least three signals have been generated, in some embodiments, the sets of signals are used to determine (usually approximately) the relationship between one or more of the Michaelis-Menten parameters and temperature.

In some embodiments the first, second, and third sets of temperature calibration parameters may include a slope and an intercept relating temperature to the value of either the first, second, or third Michaelis-Menten parameter. However, equations of other forms may be selected to relate the first, second, or third Michaelis-Menten equation to temperature. In some embodiments, a quadratic or higher-order polynomial in temperature may be suitable and/or desirable.

Other methods for estimating analyte concentration which incorporate temperature correction features and temperature calibration steps are also disclosed herein. In some embodiments, these methods are similar to those already described above and incorporate similar features, however, additional features may also be disclosed and, in some embodiments, the disclosed methods may be more general and described in more general terms. Since there are many ways to feasibly implement the discoveries disclosed herein for use in estimating analyte concentration, the following additional methods are described in order to illustrate the breadth of implementations that are possible.

In some embodiments, for instance, a method of estimating an analyte concentration from a signal indicative of the analyte concentration may include transforming the signal using an equation of the form of a modified Michaelis-Menten equation wherein the values of one or more Michaelis-Menten parameters have been adjusted for temperature.

In some embodiments, for instance, a method of estimating an analyte concentration may include generating a signal indicative of the analyte concentration and generating a signal indicative of a temperature using an analyte sensor, and transforming the signal indicative of the analyte concentration utilizing an equation of the form of a modified Michaelis-Menten equation wherein at least one of the Michaelis-Menten parameters has been substituted with a calibration equation functionally depending on a set of one or more temperature calibration parameters and the signal indicative of temperature. One could refer to such an equation as a “substituted” modified Michaelis-Menten equation since the Michaelis-Menten parameters have been explicitly substituted with equations depending on one or more other variables—temperature and the temperature calibration parameters. However, although such a “substituted” equation exhibits a more complicated analytic form, it nevertheless will still express the basic functional relationships of the modified Michaelis-Menten equation.

### Clinical Example

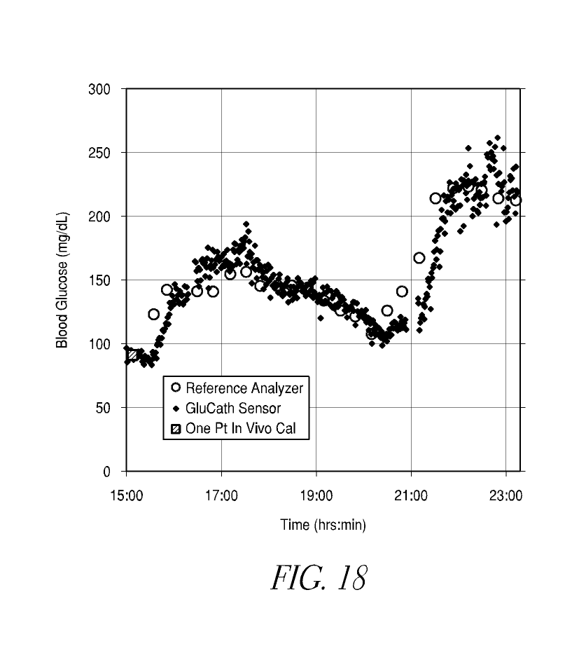

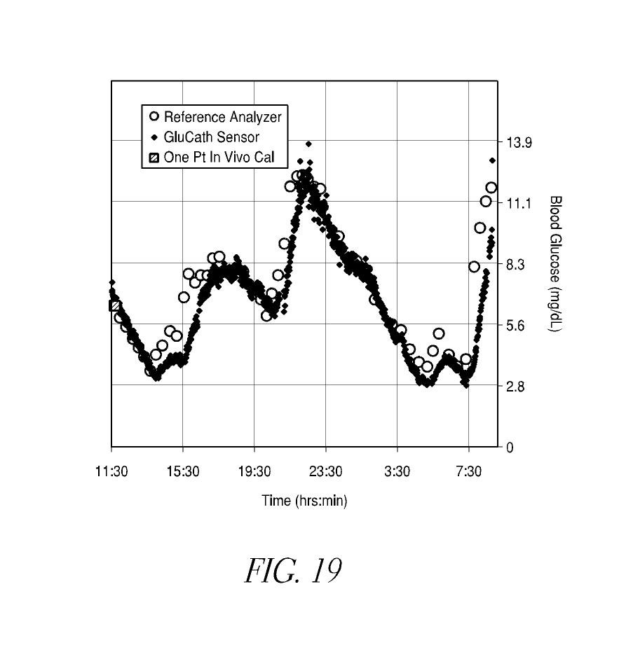

A sensor comprising a chemical indicator system was made in accordance with the present disclosure. The sensor was calibrated and prepared for deployment. The sensor was deployed into interstitial space of the subcutaneous adipose tissue in the waist area. The sensor was deployed manually using a 26 gauge split introducer. A reference measurement was taken using a laboratory glucose analyzer. An in-vivo calibration of the sensor was conducted according to the reference measurement. The sensor was thereafter allowed to continuously monitor the glucose level of the interstitial fluid in the subcutaneous adipose tissue. Reference measurements were also taken approximately every 15-60 minutes. The glucose measurement by the sensor was compared the reference measurements from the laboratory glucose analyzer. A comparison of the sensor measured values closely tracked the laboratory measured values after 8 hours of testing as shown in FIG. 18. The sensor continued to closely track the laboratory analyzer after 24 hours of continuous testing as shown in FIG. 19. The sensor also did not exhibit motion artifacts.

### Thromboresistant Coating Examples

Glucose sensors with a benzalkonium/heparin coating and 12 BD L-Cath PICC lines (outside diameter 0.037 cm, 0.0145 inches; polyurethane) as controls without coating are prepared for insertion into the cardiovascular system of four sheep. The coated glucose sensors are constructed of a fluorophore/quencher indicator system embedded in a hydrophilic acrylic matrix, as described in U.S. Patent Publication No. 2008/0187655.

Several sensors and control catheters are inserted into the interstitial space of the subcutaneous adipose tissue in the waist area of a subject. After 25 hours, some of the sensors and controls are surgically removed and examined without disturbing the sensor or catheter or any cellular accumulation or debris on the test articles or in interstitial space. After 22 additional hours (47 hours elapsed time), other sensors and controls are surgically removed and examined as described above.

Digital photographs of each sensor or catheter are taken in place. After examination, each sensor or catheter is stained with methylene blue, and examined microscopically at 10-20× primary objective power to observe build up of fibrin or cellular material or surface irregularities. Tissue sections from the veins are also obtained and characterized for the state of the tissue in proximity to the test articles.

These evaluations will demonstrate that the glucose sensor with heparin/benzalkonium coating is superior to the control catheters in terms of fewer instances of macroscopic fibrin deposits and fewer instances of microscopic fibrin deposition.

Although the foregoing invention has been described in terms of certain embodiments and examples, other embodiments will be apparent to those of ordinary skill in the art from the disclosure herein. Moreover, the described embodiments have been presented by way of example only, and are not intended to limit the scope of the inventions. Indeed, the novel methods and systems described herein may be embodied in a variety of other forms without departing from the spirit thereof. Accordingly, other combinations, omissions, substitutions and modifications will be apparent to the skilled artisan in view of the disclosure herein. Thus, the present invention is not intended to be limited by the example or preferred embodiments. The accompanying claims provide exemplary claims and their equivalents are intended to cover forms or modifications as would fall within the scope and spirit of the inventions.

What is claimed is:

1. A method for monitoring blood glucose in a subject, the method comprising:
providing a glucose sensor, comprising:
an optical fiber configured for subcutaneous deployment and capable of propagating light along a light path, and further comprising an equilibrium, non-consuming chemical indicator system disposed within the light path of the optical fiber, wherein the chemical indicator system comprises a fluorophore capable of generating a fluorescent emission signal in response to an excitation light signal, and a glucose binding moiety operably associated with the fluorophore and adapted to modify the intensity of the fluorescent emission signal in relation to the amount of glucose bound;
deploying the glucose sensor into subcutaneous tissue of the subject;
interrogating the chemical indicator system with an excitation light signal; and
detecting the intensity of the fluorescent emission light signal.

2. The method of claim 1, further comprising:
obtaining a blood sample from the subject;
measuring the glucose concentration of the blood sample independent of the chemical indicator system;
calculating a correction factor by comparing the emission light signal with the glucose concentration measured independently of the chemical indicator system; and
adjusting the blood glucose concentration measurement of the chemical indicator system with the correction factor.

3. The method of claim 1, wherein a distal end of the glucose sensor comprises an atraumatic tip portion formed from at least one material selected from the group consisting of plastics, polymers, gels, metals and composites.

4. The method of claim 3, wherein the atraumatic tip portion is configured to reduce trauma within the subcutaneous tissues and has a shape selected from the group consisting of hemispherical, parabolic, and elliptical.

5. The method of claim 1, wherein the chemical indicator system is further immobilized by a hydrogel within a gap in the optical fiber.

6. The method of claim 1, wherein the glucose binding moiety further comprises:
a viologen quencher capable of quenching the emission intensity of the fluorophore; and
a benzyl boronic acid group capable of binding glucose, wherein the benzyl boronic acid group is coupled to the viologen quencher, such that the degree of emission quenching is related to the degree of glucose binding.

7. The method of claim 1, wherein the glucose sensor further comprises a reference material, and the method further comprises;
reflecting a portion of the excitation light signal off of the reference material to generate a reflected portion of the excitation light signal; and
detecting the reflected portion of the excitation light signal.

8. The method of claim 1, wherein the glucose sensor further comprises a reference material, comprising a second fluorophore, and the method further comprises;
interrogating the reference material with the excitation light signal such that the reference material generates a second emission light signal, wherein the intensity of the second emission light signal is not related to the amount of glucose bound; and
detecting the second emission light signal.

9. The method of claim 8, wherein the reference material is encased in a glucose impermeable membrane.

10. The method of claim 1, wherein the glucose sensor further comprises a coating comprising heparin and benzalkonium is coated on a porous membrane covering the chemical indicator system.

11. The method of claim 1, further comprising:
contacting a temperature sensing element with the subcutaneous tissue of the subject, wherein the temperature sensing element is configured to generate a signal indicative of a temperature of the subcutaneous tissue of the subject;
detecting the signal indicative of a temperature of the subcutaneous tissue of the subject; and
determining a glucose concentration of the subcutaneous tissue of the subject using the detected intensity of the fluorescent emission light signal using a modified Michaelis-Menten equation comprising Michaelis-Menten parameters, wherein the Michaelis-Menten parameters are set based on data comprising,
temperature calibration data, and
the detected signal indicative of temperature.

12. The method of claim 1, further comprising:
contacting a pH sensing element with the subcutaneous tissue of the subject, wherein the pH sensing element is configured to generate a signal indicative of a pH of the subcutaneous tissue of the subject;
detecting the signal indicative of the pH of the subcutaneous tissue of the subject; and
wherein the Michaelis-Menten parameters are set based on data further comprising,
pH calibration data, and
the detected signal indicative of pH.
